(19) United States
(12) Patent Application Publication
(10) Pub. No.: US 2018/0369336 A1
(43) Pub. Date: Dec. 27, 2018

(54) B CELL-BASED CANCER IMMUNOTHERAPY

(71) Applicants: BOSTON MEDICAL CENTER CORPORATION, Boston, MA (US); TRUSTEES OF BOSTON UNIVERSITY, Boston, MA (US)

(72) Inventors: Xuemei ZHONG, Sharon, MA (US); Tyrone M. PORTER, Boston, MA (US); David C. SELDIN, Newton, MA (US); Esther LANDESMAN, Brookline, MA (US); Hung VO, Medford, MA (US); Joanna CHIU, Boston, MA (US)

(73) Assignees: BOSTON MEDICAL CENTER CORPORATION, Boston, MA (US); TRUSTEES OF BOSTON UNIVERSITY, Boston, MA (US)

(21) Appl. No.: 15/780,905

(22) PCT Filed: Dec. 2, 2016
(86) PCT No.: PCT/US2016/064575

§ 371 Date: Jun. 1, 2018

Related U.S. Application Data
(60) Provisional application No. 62/262,521, filed on Dec. 3, 2015.

(51) Int. Cl. A61K 38/19; A61K 9/00; A61P 35/00; A61K 9/127

(52) U.S. Cl. CPC A61K 38/195; A61K 9/0019; B82Y 5/00; A61K 9/1271; A61P 35/00

(57) ABSTRACT

The technology described herein is directed to cellular cancer immunotherapies involving natural IgM producing phagocytic B (NIMPAB) cells and chemokines that attract the NIMPAB cells.

### CROSS REFERENCE TO RELATED APPLICATION

This application is an International Application, which designated the U.S., and which claims the benefit under 35 U.S.C. § 119(e) of U.S. Provisional Application No. 62/262,521 filed on Dec. 3, 2015, the contents are incorporated herein by reference in their entireties.

### TECHNICAL FIELD

The technology described herein relates to the development of cellular immunotherapies involving natural IgM producing phagocytic B lymphocytes, also known as B cells (NIMPAB), and methods and compositions for enhancing NIMPAB cell numbers and tumor-infiltrating activity.

### BACKGROUND

Cancer is the second leading cause of death in the world after cardiovascular diseases, afflicting thousands of people every day throughout the world. Half of men and one third of women in the United States will develop cancer during their lifetimes. Today, although millions of cancer patients extend their life due to early identification and treatment, many more cancer patients die because of lack of effective treatment options for their individual cancers. For example, sometimes surgical removal of the cancer growth is not a viable option in cases of highly metastatic cancer, multiple tumor growths or the tumor growth is located in an inoperable area of the body. Sometimes surgery does not completely remove all the cancer cells. Other times, when even the current cancer therapy is effective and the cancer patient goes into remission, it is often temporary. The cancer patient in remission often will relapses, and the cancer resurface with a vengeance by having a faster growth rate and/or metastasis.

### SUMMARY

The technology described herein is directed to the adaptive use of a subject's innate natural IgM producing phagocytic B-1 cells (NIMPAB) for the treatment and the long-term prevention and surveillance of cancer in a cancer patient. In vivo, NIMPAB cells are naturally involved in cancer immunosurveillance and cancer cells removal.

The technology described herein is directed to the development of themosensitive or thermoresponsive nanoparticles carrying recombinant chemokines, such as CXCL13, CXCL12, and CCL19, that can be injected or retained at tumor sites in order to enhance, and/or promote the tumor infiltration of innate natural IgM producing B cells that are in circulation in the subject.

The technology described herein also relates to using phosphatidylcholine (PtC or PC) liposomes for enhancing and expanding the ex vivo cell culture of isolated NIMPAB cells. The PtC liposomes help to expand NIMPAB cell numbers, tumor-infiltrating and tumor phagocytosis activity. The resultant NIMPAB cells are then used in adoptive transfer into a subject for the treatment as a cancer therapy, and/or for the prevention of cancer.

Other aspects relate to combined cellular immunotherapies that use both the ex vivo NIMPAB expansion approaches along with intratumoral chemokine-nanoparticle injection.

Accordingly, it is the objective of this disclosure to provide a themosensitive nanoparticle carrying chemokines, such as CXCL13, CXCL12, and CCL19, that can be injected into a tumor growth or its vicinity to attract the migration of innate NIMPAB cells to the tumor microenvironment to effectual cytotoxic effects and induce growth inhibition and cell death of the tumor.

It is also the objective of this disclosure to provide a method of cancer treatment using the themosensitive nanoparticle carrying the described chemokines, such as CXCL13, CXCL12, and CCL19. One or more of the described chemokines can be used to treat cancer.

It is also the objective of this disclosure to provide a method of culture expansion of innate NIMPAB ex vivo, that is, induce mitosis of the innate NIMPAB ex vivo, thereby ensuring there is sufficient supply or more innate NIMPAB for cancer surveillance and phagocytosis of cancer cells.

It is also the objective of this disclosure to provide a method of cancer treatment using the ex vivo culture expanded of innate NIMPAB.

Accordingly, in one embodiment, this disclosure provides a nanoparticle comprising at least a lipid layer shell and an aqueous core, wherein the aqueous core comprising at least one chemokine selected from the group consisting of CXCL13, CXCL12, and CCL19, wherein the at least a lipid layer shell encapsulates the aqueous core, and wherein the at least a lipid layer shell has a phase transition temperature between 38° C. and 43° C.

In one embodiment, this disclosure provides a nanoparticle comprising a shell and an aqueous core, wherein the aqueous core comprising at least one chemokine selected from the group consisting of CXCL13, CXCL12, and CCL19, wherein the shell encapsulates the aqueous core, and wherein the shell is a temperature-responsive shell or a pH-responsive shell.

In one embodiment, this disclosure provides a composition comprising a nanoparticle comprising at least a lipid layer shell and an aqueous core, wherein the nanoparticle comprises an aqueous core comprising at least one chemokine selected from the group consisting of CXCL13, CXCL12, and CCL19, wherein the at least a first lipid layer shell encapsulates the aqueous core, and wherein the at least first lipid layer shell has a phase transition temperature between 38° C. and 43° C.

In one embodiment, this disclosure provides a composition comprising a liposome comprising an aqueous core, wherein the aqueous core comprising at least one chemokine which is selected from the group consisting of CXCL13, CXCL12, and CCL19, wherein the liposome comprises at least a first lipid layer shell that encapsulates the aqueous core, and wherein the at least first lipid layer shell has a phase transition temperature between 38° C. and 43° C.

In one embodiment, this disclosure provides a composition comprising a liposome comprising an aqueous core, wherein the aqueous core comprising at least one chemokine which is selected from the group consisting of CXCL13, CXCL12, and CCL19, wherein the liposome comprises at least a first lipid layer shell that encapsulates the aqueous core, wherein the at least a first lipid layer shell comprises of one or more of the lipid selected from the group consisting of DPPC, MPPC, PEG, DMPC, DMPG, DSPE, DOPC, POPE, DPPG, DSPC, DSPE-PEG, MSPC, cholesterol, PS, PC, PE, and/or PG; and wherein the at least first lipid layer shell has a phase transition temperature between 38° C. and 43° C.

In one embodiment of any aspect described, the chemokine is selected from the group consisting of CXCL13, CXCL12, and CCL19, and they are recombinant chemokines.

In one embodiment, this disclosure provides a composition comprising any one nanoparticle or a combination of any nanoparticles described herein or known in the art, wherein the nanoparticles comprise as aqueous core comprising at least one chemokine selected from the group consisting of CXCL13, CXCL12, and CCL19.

In one embodiment, this disclosure provides a method of treating cancer, the method comprising (a) administering a composition comprising a nanoparticle comprising at least a lipid layer shell and an aqueous core to a subject's preselected tumor or cancer target site in need of treatment for cancer, wherein the aqueous core comprising at least one chemokine selected from the group consisting of CXCL13, CXCL12, and CCL19, wherein the at least a first lipid layer shell encapsulates the aqueous core, and wherein the at least a first lipid layer shell has a phase transition temperature between 38° C. and 43° C.; and (b) heating the subject's preselected tumor target site to a temperature of between 38° C. and 45° C., whereby the chemokine in the aqueous core is released from when the environment of the nanoparticle is between 38° C. and 43° C.

In one embodiment, this disclosure provides a method of treating cancer, the method comprising administering (a) a composition described herein to a subject in need of treatment for cancer; and (b) heating a subject's preselected tumor target site to a temperature of between 38° C. and 45° C., whereby the chemokine in the aqueous core is released from the nanoparticle when the environment of the nanoparticle is between 38° C. and 43° C.

In one embodiment, this disclosure provides a method of increasing infiltration of natural IgM producing B cells in a subject to the subject's tumor target site, the method comprising administering (a) a composition described herein to the subject; and (b) heating a subject's preselected tumor/cancer target site to a temperature of between 38° C. and 45° C., whereby the chemokine in the aqueous core of the nanoparticles of the composition is released from the nanoparticle when the environment of the nanoparticle is between 38° C. and 43° C.

In one embodiment, this disclosure provides a method of preventing recurrence of cancer at a cancer exicison site, the method comprising administering (a) a composition described herein to a subject in need of prevention of cancer recurrence, wherein the composition is administered at and around the site of excision of a tumor; and (b) heating a subject's excision site to a temperature of between 38° C. and 45° C., whereby the chemokine in the aqueous core is released from the nanoparticle when the environment of the nanoparticle is between 38° C. and 43° C., wherein the composition comprising the nanoparticles or liposomes comprising chemokines, such as CXCL13, CXCL12, and CCL19, and wherein the nanoparticles or liposomes are temperature-responsive nanoparticles or liposomes. In one embodiment of this aspect, the nanoparticles or liposomes are pH-responsive and step (b) involved changing the pH at the excision site, in particularlp reducing the pH to below 7.0.

In one embodiment of any aspect described, the nanoparticle shell is permeable between the temperature 38° C. to 43° C.

In one embodiment of any aspect described, the nanoparticle shell is permeable below the pH of 7.0.

In one embodiment of any aspect described, the nanoparticle shell is a lipid layer.

In one embodiment of any aspect described, the nanoparticle shell comprises (a) one or more phospholipids selected from the group having two acyl groups, either saturated or unsaturated, and polar head group defined as phosphatidyl cholines, phosphatidyl glycerols, and/or phosphatidyl ethanolamines; (b) optionally a fatty acid or sterol with an ionizable moiety; and (c) one or more types of lysolipids selected from the group consisting of monoacylphosphatidyl cholines, monoacylphosphatidyl glycerols, and/or monoacylphosphatidyl ethanolomines.

In one embodiment of any aspect described, the lipid layer is a mixed lipid layer comprising two or more lipids.

In one embodiment of any aspect described, the mixed lipid layer comprises one or more types of phospholipids.

In one embodiment of any aspect described, the one or more types of phospholipids is/are selected from the group consisting of phosphatidyl cholines, phosphatidyl glycerols, phosphatidyl inositols and phosphatidyl ethanolamines.

In one embodiment of any aspect described, the phospholipid is selected from the group consisting of dipalmitoylphosphatidylcholine (DPPC), 1-palmitoyl-2-hydroxy-sn-glycero-3-phosphocholine (MPPC), 1-myristoyl-2-stearoyl-sn-glycero-3-phosphocholine (MSPC); 1,2-dimyristoyl-sn-glycero-3-phosphocholine (DMPC), 1,2-Dimyristoyl-sn-glycero-3-phosphorylglycerol (DMPG); 1,2-Distearoyl-sn-glycero-3-phosphoethanolamine (DSPE); 1,2-Dioleoyl-sn-glycero-3-phosphocholine (DOPC); 1,2-Dioleoyl-sn-glycero-3-phosphoethanolamine (DOPE); 1,2-dipalmitoyl-sn-glycero-3-phospho-(1′-rac-glycerol) (DPPG); 1,2-Distearoyl-sn-glycero-3-phosphocholine (DSPC); disteaoylphosphoethanolamine conjugated with polyethylene glycol (DSPE-PEG); phosphatidylserine (PS), phosphatidylethanolamine (PE), phosphatidylglycerol (PG), and phosphatidylcholine (PC).

In one embodiment of any aspect described, the one or more types of phospholipid is/are a lysolipid.

In one embodiment of any aspect described, the lysolipid is selected from the group consisting of monoacylphosphatidyl cholines, monoacylphosphatidyl glycerols, monoacylphosphatidyl inositols and/or monoacylphosphatidyl ethanolomines.

In one embodiment of any aspect described, the mixed lipid layer comprising of one or more of the lipid selected from the group consisting of DPPC, MPPC, PEG, DMPC, DMPG, DSPE, DOPC, DOPE, DPPG, DSPC, DSPE-PEG, MSPC, cholesterol, PS, PC, PE, and/or PG.

In one embodiment of any aspect described, the nanoparticle is a liposome comprising at least a first lipid bilayer. In one embodiment, the at least a first lipid bilayer comprises one or more of the lipid selected from the group consisting of DPPC, MPPC, PEG, DMPC, DMPG, DSPE, DOPC, DOPE, DPPG, DSPC, DSPE-PEG, MSPC, cholesterol, PS, PC, PE, and/or PG. In one embodiment, the nanoparticle is any liposome made with one or more of the lipid selected from the group consisting of DPPC, MPPC, PEG, DMPC, DMPG, DSPE, DOPC, DOPE, DPPG, DSPC, DSPE-PEG, MSPC, cholesterol, PS, PC, PE, and/or PG. In one embodiment, the nanoparticle is any liposome known in the art.

In one embodiment of any aspect described, the nanoparticles have a selected mean particle size of less than or equal to 150 nm.

In one embodiment of any aspect described, the nanoparticles have a selected mean particle size of between 60-150 nm.

In one embodiment of any aspect described, the mixed lipid layer comprises of 5-20 mol % of MPPC or MSPC.

In one embodiment of any aspect described, the mixed lipid layer comprises of 5-18 mol % of MPPC or MSPC.

In one embodiment of any aspect described, the mixed lipid layer comprises of 8.5-10 mol % of MPPC or MSPC.

In one embodiment of any aspect described, the mixed lipid layer comprises of 85-95 mol % of DPPC or DPPG.

In one embodiment of any aspect described, the mixed lipid layer comprises of 0.1-10.0 mol % of DSPE-PEG.

In one embodiment of any aspect described, the mixed lipid layer comprises of no more that 4 mol % of DSPE-PEG.

In one embodiment of any aspect described, the mixed lipid layer consists essentially of 5-20 mol % of MPPC or MSPC.

In one embodiment of any aspect described, the mixed lipid layer consists essentially of 5-18 mol % of MPPC or MSPC.

In one embodiment of any aspect described, the mixed lipid layer consists essentially of 8.5-10 mol % of MPPC or MSPC.

In one embodiment of any aspect described, the mixed lipid layer consists essentially of 85-95 mol % of DPPC or DPPG.

In one embodiment of any aspect described, the mixed lipid layer consists essentially of 0.1-10.0 mol % of DSPE-PEG.

In one embodiment of any aspect described, the mixed lipid layer consists essentially of no more that 4 mol % of DSPE-PEG.

In one embodiment of any aspect described, the DSPE-PEG is DSPE-PEG2000.

In one embodiment of any aspect described, the mixed lipid layer forms a lipid bilayer comprising of (a) DPPC or DPPG; (b) MPPC or MSPC; and (c) DSPE-PEG.

In one embodiment of any aspect described, the mixed lipid layer forms a lipid bilayer comprising of DPPC, MPPC and DSPE-PEG.

In one embodiment of any aspect described, the mixed lipid layer forms a lipid bilayer comprising of DOPE, a fatty acid or sterol with an ionizable moiety, and DSPE-PEG.

In one embodiment of any aspect described, the mixed lipid layer forms a lipid bilayer comprising of DPPC, MPPC and DSPE-PEG in the molar ratio 90:10:4.

In one embodiment of any aspect described, the nanoparticle comprises a second inner layer of mixed lipid which encapsulates the aqueous core comprising of the chemokine.

In one embodiment of any aspect described, the second layer of mixed lipid comprises of (a) DPPC or DPPG; and (b) MPPC or MSPC.

In one embodiment of any aspect described, the second layer of mixed lipid comprises of DPPC and MPPC.

In one embodiment of any aspect described, the second layer of mixed lipid comprises of DPPC and MPPC in the molar ratio 90:10.

In one embodiment of any aspect described, the nanoparticle is a temperature-responsive liposome wherein the chemokine in the aqueous core is released from the nanoparticle when the environment of the nanoparticle is between 38° C. and 43° C.

In one embodiment of any aspect described, at least 70% of the chemokine is released when the environment of the nanoparticle is between 38° C. and 43° C.

In one embodiment of any aspect described, the chemokine is released within 5 minutes when the environment of the nanoparticle is between 38° C. and 43° C.

In one embodiment of any aspect described, the nanoparticle is a temperature-responsive liposome wherein at least 70% of the chemokine in the aqueous core is released within 5 minutes when the environment of the nanoparticle is between 38° C. and 43° C.

In one embodiment of any aspect described, the aqueous core comprises only one chemokine.

In one embodiment of any aspect described, the aqueous core comprises only two chemokines, the two-chemokine combination is selected from the group consisting of CXCL13 and CXCL12; CXCL13 and CCL19; and CXCL12 and CCL19.

In one embodiment of any aspect described, the aqueous core comprises all three chemokines CXCL13, CXCL12, and CCL19.

In one embodiment of any aspect described, the aqueous core further comprises a fluorescent dye or radioactive dye.

In one embodiment of any aspect described, the composition further comprising at least one pharmaceutically acceptable carrier, diluent, excipient or adjuvant.

In one embodiment of any aspect described, the composition further comprising a thermosensitive magnetic liposome (TSML).

In one embodiment of any aspect described, the treatment method comprises the infiltration of NIMPAB cells to the tumor/cancer target site and the infiltration is increased by administration of the composition described which is followed by the heating at the targeted site. The targeted sites are pre-selected.

In one embodiment of any aspect described, the treatment method comprises the released chemokine at the tumor/cancer target site to promotes in vivo infiltration of the subject's own NIMPAB cells to the tumor/cancer target site.

In one embodiment of any aspect described, the treatment method comprises that the preselected tumor target site is a solid tumor.

In one embodiment of any aspect described, the treatment method comprises the administration by direct intratumoral injection.

In one embodiment of any aspect described, the method of administration is by parenteral, oral, buccal, pulmonary, intravenous, intramuscular, subcutaneous, aural, rectal, vaginal, ophthalmic, intradermal, intraoccular, intracerebral, intralymphatic, intraarticular, intrathecal or intraperitoneal injection.

In one embodiment of any aspect described, the heating of step (b) in the treatment method is by high intensity focused ultrasound (HIFU) which allows non-invasive heating to establish hyperthermia (40-45° C.) of tumor/cancer target site over time.

In one embodiment of any aspect described, the subject is a mammal.

In one embodiment of any aspect described, the mammal subject is a primate mammal.

In one embodiment of any aspect described, the mammal is a human.

In one embodiment, this disclosure provides a method of expanding and/or stimulating NIMPAB cells derived from a subject, the method comprising culturing an isolated population of NIMPAB cells from a subject with a liposome comprising phosphatidylcholine (PC, also abbreviated PtC) and/or a composition comprising a liposome comprising PC for a period of time under culture conditions that promotes the expansion of the initial population of NIMPAB cells.

In one embodiment of any aspect described, the NIMPAB cells are phagocytic B cells.

In one embodiment of any aspect described, the NIMPAB cells are B-1 type B lymphocytes or B cells.

In one embodiment of any aspect described, the NIMPAB cells are phagocytic B-1 cells

In one embodiment of any aspect described, the NIMPAB cells are phagocytic L2pB1 cells.

In one embodiment of any aspect described, the culturing is ex vivo.

In one embodiment of any aspect described, the cell expansion method further comprising providing a sample of peritoneal cavity cells from the subject, wherein the sample comprises NIMPAB cells.

In one embodiment of any aspect described, the cell expansion method further comprising isolating a population of NIMPAB cells from a subject.

In one embodiment of any aspect described, the cell expansion method further comprising isolating a population of natural IgM producing B cells from a sample of peritoneal cavity cells from a subject. In one embodiment of any aspect described, the cell expansion method further comprising isolating a population of NIMPAB cells from a sample of peritoneal cavity cells from a subject.

In one embodiment of any aspect described, the cell expansion method further comprising selecting for natural IgM producing B cells from the subject prior to the ex vivo culturing. In one embodiment of any aspect described, the cell expansion method further comprising selecting for NIMPAB cells from the subject prior to the ex vivo culturing. For example, selecting for natural IgM producing B cells or NIMPAB cells from the sample of peritoneal cavity cells obtained from the subject.

In one embodiment of any aspect described, the cell expansion method further comprising selecting for natural IgM producing B cells from the cell culture after the ex vivo culturing and expansion. In one embodiment of any aspect described, the cell expansion method further comprising selecting for NIMPAB cells from the cell culture after the ex vivo culturing and expansion.

In one embodiment of any aspect described, the cell expansion method further comprising harvesting for natural IgM producing B cells from the cell culture after the ex vivo culturing. In one embodiment of any aspect described, the cell expansion method further comprising harvesting for NIMPAB cells from the cell culture after the ex vivo culturing.

In one embodiment of any aspect described, the cell expansion method further comprising cryopreservation of the harvested natural IgM producing B cells prior to use. In one embodiment of any aspect described, the cell expansion method further comprising cryopreservation of the harvested NIMPAB cells prior to use.

As used herein, “cryopreservation” refers to the preservation of cells by cooling to low sub-zero temperatures, such as (typically) 77 K or 196° C. (the boiling point of liquid nitrogen). Cryopreservation also refers to storing the cells at a temperature between 0° C.-10° C. in the absence of any cryopreservative agents. At these low temperatures, any biological activity, including the biochemical reactions that would lead to cell death, is effectively stopped. Cryoprotective agents are often used at sub-zero temperatures to preserve the cells from damaged due to freezing at low temperatures or warming to room temperature.

In one embodiment, this disclosure provides a method of treating cancer, the method comprising administering a population of ex vivo culture expanded NIMPAB cells to a subject in need of treatment for cancer, wherein the NIMPAB cells are culture expanded by any method described.

In one embodiment, this disclosure provides a method of treating cancer in a subject in need of cancer treatment, the method comprising (a) culturing an initial population of NIMPAB cells with a liposome comprising phosphatidylcholine (PC) or a composition comprising a liposome comprising PC or both for a period of time under culture conditions that promotes the expansion of the initial population of NIMPAB cells; (b) culturing the cell ex vivo; and (c) administering the harvested cells to a recipient subject in need of treatment for cancer.

In one embodiment of any aspect described, the treatment method further comprising providing a sample of peritoneal cavity cells from a donor subject, wherein the sample comprises NIMPAB cells.

In one embodiment of any aspect described, the treatment method further comprising a step of selecting for the expanded NIMPAB cells prior to administering the cell to the recipient subject.

In one embodiment of any aspect described, the treatment method further comprising a step of harvesting for expanded NIMPAB cells prior to administering the cell to the recipient subject.

In one embodiment of any aspect described, the treatment method further comprising a step of enriching for expanded NIMPAB cells prior to administering the cell to the recipient subject.

In one embodiment of any aspect described, the treatment method further comprising a step of cryopreserving the expanded NIMPAB cells prior to administering the cell to the recipient subject.

In one embodiment of any aspect of the treatment method described, the NIMPAB cells are phagocytic B cells.

In one embodiment of any aspect of the treatment method described, the NIMPAB cells are B-1 type B lymphocytes.

In one embodiment of any aspect of the treatment method described, the NIMPAB cells are phagocytic B-1 cells

In one embodiment of any aspect of the treatment method described, the NIMPAB cells are phagocytic L2pB1 cells.

In one embodiment of any aspect of the treatment method described, the NIMPAB cell is obtained from a healthy donor subject.

In one embodiment of any aspect of the treatment method described, the NIMPAB cell is obtained from peripheral blood; through hemodialysis; from the peritoneal cavity; through peritoneal dialysis; or from a tumor sample.

In one embodiment of any aspect of the treatment method described, the donor subject and the recipient subject are not the same subject.

In one embodiment of any aspect of the treatment method described, the NIMPAB cell is non-autologous to the recipient subject.

In one embodiment of any aspect of the treatment method described, the non-autologous NIMPAB cell is at the minimum HLA match with the recipient subject.

In one embodiment of any aspect of the treatment method described, the donor subject and the recipient subject are the same subject.

In one embodiment of any aspect of the treatment method described, the NIMPAB cell is autologous to the recipient subject.

As used herein, the term “autologous” refers to a situation in which the donor of the NIMPAB cells for ex vivo expansion and recipient of the expanded NIMPAB cells are the same person.

In one embodiment of any aspect of the treatment method described, the administration is by direct intratumoral injection.

In one embodiment of any aspect of the treatment method described, the method of administration is by parenteral, oral, buccal, pulmonary, intravenous, intramuscular, subcutaneous, aural, rectal, vaginal, ophthalmic, intradermal, intraoccular, intracerebral, intralymphatic, intraarticular, intrathecal or intraperitoneal injection.

In one embodiment of any aspect of the treatment method described, the subject is a mammal.

In one embodiment of any aspect of the treatment method described, the mammal subject is a primate mammal.

In one embodiment of any aspect of the treatment method described, the mammal is a human.

### Definitions

Natural IgM antibodies (Abs) are the circulating IgM Abs that arise without known immune exposure or vaccination are referred to as natural, whereas immune IgM are generated in response to defined antigenic stimuli. Natural IgM Abs are generated by natural gene assortment in the cells and are part of the innate immune system in an organism.

Natural IgM-producing cell are the B lymphocytes that produces the natural IgM Abs.

As used herein, natural IgM producing B cells, natural IgM producing phagocytic B cells, and NIMPAB cells are used interchangeably to refer to natural IgM producing phagocytic B-1 type B lymphocytes/B cells.

Lysolipids or lysophospholipid is any derivative of a phospholipid in which one or both acyl derivatives have been removed by hydrolysis. Examples include phosphatidylcholine (PtC) and phosphatidylethanolamine (PE).

As used herein in reference to the nanoparticles or liposomes, the term “temperature-responsive” or “thermosensitive” refers to the wall or shell of the nanoparticle or liposome becoming permeable between the temperature 38° C. to 43° C.

As used herein in reference to the nanoparticles or liposomes, the term “pH-responsive” refers to the wall or shell of the nanoparticle or liposome becoming permeable below the pH of 7.0.

### BRIEF DESCRIPTION OF THE DRAWINGS

FIGS. 1A-1C demonstrate human phagocytic B cells in peripheral blood. Peripheral PBMC was obtained from healthy donor and incubated over night with PtC-nanoparticle and control nanoparticles at 1:20 ratio. Cells were then stained for CD19, CD20 (FIG. 1A) and CD5 and CD14 (FIG. 1B). In this case, 15% of human CD19+CD20+ B cells specifically phagocytose PtC-nanoparticles, whereas CD14+ monocytes phagocytose both PtC-nanoparitcles and control nanoparticles (FIG. 1C). Results are representative of four experiments.

FIGS. 2A-2C demonstrate that L2pB1 cells are the predominant NIMPAB in mouse.

FIG. 2A depicts flow cytometry demonstrating that NBD (green fluorescence) is embedded in liposomes as pH-insensitive reference fluorescence. pHrodo-dye on the surface of the liposomes were used as indicator of pH reduction in the event of phagolysome formation. Double positive signals of red (pHrodo) and green (NBD) indicate phagocytosis of the liposomes (see arrow) while single positive signal of green (NBD) without red signified surface attachment of the liposomes. Peritoneal cells were incubated with either PtC-liposomes or control liposomes for 2 hours at 37° C. FACS analysis of both pHrodo red and NBD green signals in B-1 cells (CD19+, IgM+, CDS+, CD11b-mid) is shown as indicator of acidic phagolysosome formation.

FIG. 2B depicts flow cytometry in which PD-L2 expression of B-1 cells and phagocytosis of PtC-liposome is analyzed. All PtC-liposome-containing cells are PD-L2 positive (red arrow) whereas none of the PD-L2 negative B1 cells contains PtC-liposome.

FIG. 2C shows the phase contrast of a B-1 cell having internalized PtC-liposomes by phagocytosis. The liposomes location in the cell is indicated by an arrow. An illustration of the design of PtC-liposomes is shown at lower right corner.

FIGS. 3A-3F demonstrate that mouse peritoneal cells induce lipoptosis of melanoma cells. Melanoma cells of the B16F10 cell line were incubated alone (FIGS. 3A, 3C, 4E) or with peritoneal cells (FIGS. 3B, 3E, 3F). Peritoneal cells contained more than 50% of B-1 cells. Bright field (FIGS. 3A, 3B), oil red O staining and fluorescent image (FIGS. 3C,3D) and oil red O combined with hematoxylin staining (FIGS. 3E, 3F) are shown. Oil red O staining indicates accumulated lipids in dying cancer cells, a feature of lipootosis.

FIGS. 4A-4E demonstrate that L2pB1 cells are required for inhibiting B16F10 melanoma cells. FIG. 4A depicts flow cytometry results. CD19-Cre-PZTD mice were injected with Diphtheria toxin (DT) to deplete L2pB1 cells. An almost complete loss of PD-L2+ B-1 B cells (PD-L2+TdTomato+) was shown. FIG. 4B depicts images of B16F10 melanoma cells cultured alone for 3 days. FIG. 4C depicts an image of B16F10 melanoma cells co-cultured with wild type peritoneal cells. Most B16F10 cells were dying by day 3. FIG. 4D depicts an image of B16F10 melanoma cells co-cultured with total spleen cells. No significant inhibition was observed. FIG. 4E depicts an image of B16F10 melanoma cells co-cultured with L2pB1 cell-depleted peritoneal cells. B16F10 melanoma cell inhibition and cell death were significantly diminished as compared to control peritoneal cells in FIG. 4C.

FIGS. 5A-5C demonstrate that L2pB1 cells are the predominant peritoneal B lymphocytes that constitutively produce IL-10. FIG. 5A depicts flow cytometry results. Peritoneal cells were obtained from IL-10-GFP knock in C57BL/6 mice. Cells were then cultured in the presence or absence of 5 ug/ml LPS overnight followed by FACS analysis. IL-10 expression was monitored through GFP expression. L2pB1 cells were gated as Mac-1+B220lowIgM+PD-L2+ cells. L2nB1 cells were gated as Mac-1+B220lowIgM+PD-L2− cells. GFP expression of L2pB1 and L2nB1 cells were compared. No GFP expression was observed in L2nB1 cells in untreated cells. Low level of GFP expression was observed in L2pB1 cells without LPS stimulation. After LPS stimulation, there is a significant increase of GFP expression in L2pB1 cells. GFP expression was also observed in L2nB1 cells after LPS stimulation. But L2pB1 cells express much higher level of GFP than L2nB1 cells. No GFP expression was observed in T cells and B2 B cells in the peritoneal cavity (data not shown). FIGS. 5B-5C depict graphs of the quantified results.

FIG. 6 depicts a schematic of the anti-cancer functions of NIMPAB as exemplified by L2pB1 cells in mice. (1) Unlike other immune cells, NIMPAB cells can self-renewal and do not require continuous replenishment from BM stem cells; (2) NIMPAB cells secret broad-cancer-recognizing natural IgM (nIgM) antibodies; (3) NIMPAB-derived nIgM can induce lipoptosis of cancer cells by binding to both lipids and cancer cells and over-feeding cancer cells with lipids upon internalization; (4) Apoptotic bodies or microvesicles from dying cancer cells can further activate NIMPAB cells to differentiate into larger phagocytic cells; (5) NIMPAB cells further engulf cancer cells; (6) NIMPAB cells process tumor antigens upon phagocytosis of cancer cells; (7) NIMPAB cells secret GM-CSF to recruit other cancer-fighting cells like macrophage, DC and NKT cells; (8) NIMPAB cells secret anti-inflammatory cytokine IL-10 to control local inflammation; (9) Suppressive Treg cells and exhausted T cells that express high level of PD-1 (receptor for PD-2) will be inhibited by PD-L2 on NIMPAB cells; (10) Only T cells that can recognize tumor antigen and have no or low expression of PD-1 will be activated by NIMPAB cells. (11) NIMPAB-activated T cells and other immune cells (12) will jointly control more cancer cells.

FIG. 7 depicts a schematic of NIMPAB-based cancer immunotherapy. Step 1: CD5+CD27+IgM+ PtC-nanoparticle-binding NIMPAB cells will be isolated from surgically removed tumor, peripheral blood or peritoneal cavity by apheresis from patient before chemotherapy or from healthy donors. Step2: These cells will be further enriched, activated and expanded in vitro by PtC-liposomes. If tumor antigens are known, they will be incorporated into the PtC-liposome. Step 3: The resulting NIMPAB cells will then be transferred back to patients i.v. or by local intratumoral injection. Step 4: Nanoparticles carrying NIMPAB-attracting chemokines will be injected into the tumor to promote migration and infiltration of NIMPAB cells into the tumor. Slow releasing of the chemokines from the nanoparticles will stabilize tumor-infiltrating NIMPAB inside tumor for prolonged anti-tumor effects.

FIGS. 8A-8B depict the PD-L2-ZsGreen-TdTomato-Diphtheria Toxin Receptor KI-KO (PZTD) mouse model.

FIG. 8A depicts a schematic of cloning A cDNA copy of ZsGreen, a green fluorescent protein was inserted after the stop codon in exon 5 of PD-L2 gene separated by an internal ribosome entry site (IRES). A Neomycin resistant gene (Neo) and a BGHPA sequences were inserted after exon 6. All these insertions were flanked by two LoxP sequences (triangles). A duplication of exon 5 was inserted after the 3′ end LoxP sequence. An IRES and a cDNA copy of Diphtheria toxin receptor (DTR) were inserted after the stop codon in the duplicate exon 5 followed by a cDNA copy of red fluorescent protein, TdTomato. DTR and TdTomato genes were separated by a 2A sequence. After crossing to a Cre recombinase transgenic mouse, the sequence flanked by LoxP sites will be removed permanently, leaving only IRES-DTR-2A-TdTomato sequence.

FIG. 8B depicts the results of Peritoneal B-1 B cells from WT, PZTD and PZTD mice crossed to CD19-Cre KI mice analyzed by FACS. The various arrows indicate the WT L2pB1 cells in heterozygous PZTD mice, the L2pB1 cells that express ZsGreen in PZTD mice, and the L2pB1 cells in PZTD mice after crossing to CD19-Cre KI mice.

FIG. 9 depicts a schematic of intratumoral injection of chemokine-nanoparticle and migration of B-1 B cells out of body cavities.

FIG. 10 depicts a schematic of an animal model for NIMPAB-based treatment. Step1 is the subcutaneous (s.c.) inoculation of the primary tumor (green dot), for example melanoma cells followed by intraperitoneal (i.p.) adoptive transfer of L2pB1 cells and intratumoral (i.t.) injection of chemokine-nanoparticles. Step 2 is the introduction of a secondary tomor that is of a different origin from the primary tumor, for example, brain tumor or lung carcinoma cells. A successful treatment would result in the shrinkage and remission of the primary tumor (green dot) and the prevention of the growth of the secondary tumor.

FIG. 11 shows that PCW cells but not splenocytes inhibit syngeneic melanoma tumor growth in an in vitro 3D tumor spheroid growth model.

FIG. 12A-12B show that depletion of L2pB1 cells in vivo in mice resulted in enlarged tumor sizes.

FIG. 12A shows melanoma tumor formation in CD19-Cre-PZTD mice that either received i.p. injection of DT or PBS before tumor inoculation. Mice received DT injection showed 70-80% depletion of L2pB1 cells at the end of the experiment. Tumors formed in DT-treated mice showed larger size and more engiogenesis.

FIG. 12B showed statistical difference in tumor size in DT and PBS-treated mice.

FIG. 13A shows that the transwell experimental design and FACS data demonstrating that B1a cells were preferably attracted by low concentration of CXCL13 in the transwell experiment.

FIG. 13B shows that PCW cells migrate towards CXCL13.

FIG. 13C shows that CXCL13 mobilize large B1a cells.

FIG. 13D shows that CXCL13 attracts more large L2pB1 cells than L2nB1 cells.

FIG. 14 shows that CXCL13-carrying temperature sensitive liposomes (TSL) can attract PCW cells upon heating.

### DETAILED DESCRIPTION

Unless otherwise defined herein, scientific and technical terms used in connection with the present application shall have the meanings that are commonly understood by those of ordinary skill in the art to which this disclosure belongs. It should be understood that this disclosure is not limited to the particular methodology, protocols, and reagents, etc., described herein and as such can vary. The terminology used herein is for the purpose of describing particular embodiments only, and is not intended to limit the scope of the disclosure, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3) (2015 digital online edition at internet website of Merck Manuals); Robert S. Porter et al. (eds.), The Encyclopedia of Molecular Cell Biology and Molecular Medicine, published by Blackwell Science Ltd., 1999-2012 (ISBN 9783527600908); Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Current Protocols in Molecular Biology (CPMB), Frederick M. Ausubel (ed.), John Wiley and Sons, 2014 (ISBN 047150338X, 9780471503385), and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents is based on the information available to the applicants and does not constitute any admission as to the correctness of the dates or contents of these documents.

Embodiments of the technology described herein are based on the observation of the innate immune system in an organism, such as a mammal, which naturally produced antibodies (Abs) to cancerous cells. For example, all humans generate abnormal or precancerous cells on a regular basis. However, only a very small percentage of the general population develops cancer. This is because in healthy humans the immune system constantly generates potent antibodies that seek out and kill abnormal or precancerous cells before large tumors are formed. In contrast, the elderly, who often suffer from compromised immunity, have a significantly higher rate of cancer compared to other segments of the population. The process whereby the immune system is constantly screening and removing pre-cancerous cells is known as cancer immuno-surveillance. Immuno-surveillance differs from conventional immune response in that it does not launch systemic inflammation, it is an ongoing maintenance process that is not terminated in a short term and more importantly it has a broad-spectrum cancer recognition mechanism.

Over 99% of the anti-cancer antibodies generated by the human immune system are of the IgM subclass. Natural IgM Abs are the constitutively secreted products of B1 cells (CD5⁺ in mice and CD20⁺CD27⁺CD43^{+/-}CD70⁻ in humans) that have important and diverse roles in health and disease. Whereas the role of natural IgM as the first line of defense for protection against invading microbes has been extensively investigated, more recent reports have highlighted their potential roles in the maintenance of tissue homeostasis via clearance of apoptotic and altered cells through complement-dependent mechanisms, inhibition of inflammation, removal of misfolded proteins, and regulation of pathogenic autoreactive IgG Abs and autoantibody-producing B cells. These observations have provided the theoretical underpinnings for efforts that currently seek to harness the untapped therapeutic potential of natural IgM in cancer treatment and also prevention of relapse.

In health, the circulating IgM that arise without known immune exposure or vaccination are referred to as natural, whereas immune IgM are generated in response to defined antigenic stimuli. In the mouse, natural IgM or innate natural (nIgM) are often without N-region additions and are germline encoded or with minimal somatic hypermutations. nIgM can display polyreactivity, whereas some IgM clones have highly refined antigen-binding (Ag-binding) specificities. In mice, B-1 lymphocytes represent a unique B-cell population distinguished from follicular B cells (B-2 cells) and marginal zone B cells by their surface marker expression, developmental origin, self-renewing capacity, and functions. These B-1 cells are identified by cell surface expression of IgM^{hi}, IgD^{lo}, CD23⁻, CD43⁺, and B220^{lo}. The vast majority is found in peritoneal and pleural cavities, whereas almost no B-1 cells are found in the peripheral blood and lymphoid tissues. B-1 cells are responsible for the production of so-called natural antibodies that occur spontaneously in naive “pathogen-free” mice. These are polyspecific antibodies of low affinity and predominantly of the IgM isotype. They constitute a first line of defense against microbial antigens. These B-1 cells are the major source of nIgM. In humans, the CD20⁺CD27⁺CD43⁺CD70⁻ B-1 cells are believed to produce the natural IgM (nIgM) in the same manner as the mouse B-1 B cells as oppose to immune or activated IgM.

These B-1 cells are distinct from CD20⁺CD27⁺CD43⁻ activated memory B and CD20⁺CD27⁻CD43⁻ naive B cells because when activated, B-1 do not display the activation markers CD69 and CD70. B-1 cells are also characterized by their ability to efficiently present antigens and can provide potent signaling to T cells in the absence of specific antigenic stimulus. Compared to B-2 cells that are susceptible to BCR-mediated negative selection due to activation-induced apoptotic death, B-1 cells are resistant to strong BCR-mediated signaling. B-1 cells also have a special ability for self-renewal that ensures the continuous production of nIgM throughout life, even though the bone marrow can also generate some subtype of B-1 B cells under certain circumstance.

The functional contributions of B-1 cells are intertwined in the tight regulation of their trafficking between different lymphoid compartments. B-1 cells migrate from the bone marrow and into the peritoneal cavity as they follow gradients of chemokines such as CXCL13, CXCL12 and CCL19. It has been shown that CXCL13-deficient mice have reduced levels of peritoneal B-1 cells. Stimulation with certain cytokines, or by infectious agent-derived ligands that bear the pathogen-associated molecular patterns (PAMPs), recognized by innate immune receptors, such as TLRs, can activate peritoneal B-1 cells. This process can also induce expression of the chemokine receptor CCR7 that can mediate their relocalization to other lymphoid organs and differentiation into Ig-producing cells. However, the homing of B1 cells into the peritoneal cavity is not an absolute requirement for mounting T-independent Ab responses, as most of the production of IgM by murine B-1 cells occurs in the spleen.

Most cancer immunotherapy strategies that try to launch conventional immune responses against specific tumor antigen or signal pathway have limited success due to lack of adaptation to cancer variation and short-lived effects. Described herein is an exploitation of the innate cancer immunosurveillance mechanism involving natural IgM producing B cells to provide a novel robust, broad-spectrum and self-sustainable cancer therapy. The cancer therapy uses nanoparticles carrying the appropriate chemokines to attact the natural IgM producing B cells to the target cancer location. In addition, the cancer therapy uses a method of increasing the number of the natural IgM producing B cells for cancer immunosurveillance.

### Chemokine-Carrying Nanoparticles and Therapeutic Uses Thereof

Accordingly, in one embodiment, this disclosure provides a nanoparticle comprising at least a first lipid layer shell and an aqueous core, wherein the aqueous core comprising at least one chemokine selected from the group consisting of CXCL13, CXCL12, and CCL19, wherein the at least first lipid layer shell encapsulates the aqueous core, and wherein the at least a lipid layer shell has a phase transition temperature between 38° C. and 43° C. In one embodiment, the aqueous core of the nanoparticle further comprises GM-CSF.

In one embodiment, this disclosure provides a nanoparticle comprising a shell and an aqueous core, wherein the aqueous core comprising at least one chemokine selected from the group consisting of CXCL13, CXCL12, and CCL19, wherein the shell encapsulates the aqueous core, and wherein the shell is a temperature-responsive shell or a pH-responsive shell. In one embodiment, the aqueous core of the nanoparticle further comprises granulocyte-macrophage colony-stimulating factor (GM-CSF).

In one embodiment, this disclosure provides a composition comprising a nanoparticle comprising at least a lipid layer shell and an aqueous core, wherein the nanoparticle wherein the aqueous core comprising at least one chemokine selected from the group consisting of CXCL13, CXCL12, and CCL19, wherein the at least a lipid layer shell encapsulates the aqueous core, and wherein the at least a lipid layer shell has a phase transition temperature between 38° C. and 43° C. In one embodiment, the aqueous core of the nanoparticle further comprises GM-CSF. In one embodiment, the composition further comprises GM-CSF.

In one embodiment, this disclosure provides a composition comprising a nanoparticle comprising a shell and an aqueous core, wherein the aqueous core comprising at least one chemokine selected from the group consisting of CXCL13, CXCL12, and CCL19, wherein the shell encapsulates the aqueous core, and wherein the shell is a temperature-responsive shell or a pH-responsive shell. In one embodiment, the aqueous core of the nanoparticle further comprises GM-CSF. In one embodiment, the composition further comprises GM-CSF.

In one embodiment, this disclosure provides a composition comprising any one nanoparticle or a combination of any nanoparticles described herein. In one embodiment, this disclosure provides a composition comprising a nanoparticle comprising a liposome comprising at least a first lipid bilayer comprising of one or more of the lipid selected from the group consisting of DPPC, MPPC, PEG, DMPC, DMPG, DSPE, DOPC, DOPE, DPPG, DSPC, DSPE-PEG, MSPC, cholesterol, PS, PC, PE, and/or PG. In one embodiment, the nanoparticle is a liposome made of one or more of the lipid selected from the group consisting of DPPC, MPPC, PEG, DMPC, DMPG, DSPE, DOPC, DOPE, DPPG, DSPC, DSPE-PEG, MSPC, cholesterol, PS, PC, PE, and/or PG. In one embodiment, this disclosure provides a composition comprising (a) a liposome comprising at least a first lipid bilayer comprising of one or more of the lipid selected from the group consisting of DPPC, MPPC, PEG, DMPC, DMPG, DSPE, DOPC, DOPE, DPPG, DSPC, DSPE-PEG, MSPC, cholesterol, PS, PC, PE, and/or PG; and (b) an aqueous core, wherein the aqueous core comprising at least one chemokine selected from the group consisting of CXCL13, CXCL12, and CCL19, wherein the at least first lipid layer shell encapsulates the aqueous core, and wherein the at least a lipid layer shell has a phase transition temperature between 38° C. and 43° C. In one embodiment, the composition further comprises a pharmaceutically acceptable carrier. In one embodiment, the composition further comprises an additional cancer therapeutic agent. In one embodiment, the composition further comprises a pharmauetically acceptable carrier and an additional cancer therapeutic agent. In one embodiment, the aqueous core of the nanoparticle further comprises GM-CSF. In one embodiment, the composition further comprises GM-CSF.

In one embodiment of any aspect described, the chemokines selected from the group consisting of CXCL13, CXCL12, and CCL19, and they are recombinant chemokines.

### Chemokines for Promoting Migration of Natural IgM Producing B Cells

Chemokines are chemotactic cytokines, of molecular weight 6-15 kDa, that are released by a wide variety of cells to attract and activate, among other cell types, macrophages, T and B lymphocytes, eosinophils, basophils and neutrophils. There are four classes of chemokines, CXC, CC, C, and CX3C, depending on whether the first two cysteines in the amino acid sequence are separated by a single amino acid (CXC) or are adjacent (CC).

Chemokines bind to specific cell-surface receptors belonging to the family of G-protein-coupled seven-transmembrane-domain proteins, which are termed “chemokine receptors.” On binding to their cognate ligands, chemokine receptors transduce an intracellular signal though the associated trimeric G proteins, resulting in, among other responses, a rapid increase in intracellular calcium concentration, changes in cell shape, increased expression of cellular adhesion molecules, degranulation, and promotion of cell migration.

It has been shown that B-1 cells migrate from the bone marrow and into the peritoneal cavity as they follow gradients of CXCL13, and CXCL13-deficient mice have reduced levels of peritoneal B-1 cells.

Chemokine (C—X—C motif) ligand 13 (CXCL13) also known as B lymphocyte chemoattractant (BLC) is a protein ligand that in humans is encoded by the CXCL13 gene.

CXCL13 is a small cytokine belonging to the CXC chemokine family. As its name suggests, this chemokine is selectively chemotactic for B cells belonging to both the B-1 and B-2 subsets, and elicits its effects by interacting with chemokine receptor CXCR5. In mouse, B-1 cell express higher CXCR5 than B-2 cells. Thus, CXCL13 attracts B-1 cells more than B-2 cells. CXCL13 and its receptor CXCR5 control the organization of B cells within follicles of lymphoid tissues, and is expressed highly in the liver, spleen, lymph nodes, and gut of humans. The gene for CXCL13 is located on human chromosome 4 in a cluster of other CXC chemokines.

Recombinant CXCL13 protein can be obtained commercially, for example, from R&D Systems, Novus Biologicals (product #P3589), Biolegend, and Peprotein. Alternative, standard molecular biology techniques can be used to express a recombinant CXCL13 protein. One skilled in the art would be to clone and synthesize recombinant protein. For example, as described in U.S. Patent Application publication No: 20140045211, the contents are herein incorporated by reference in their entirety.

The stromal cell-derived factor 1 (SDF-1) also known as C—X—C motif chemokine 12 (CXCL12) is a chemokine protein that in humans is encoded by the CXCL12 gene. Stromal cell-derived factors 1-alpha and 1-beta are small cytokines that belong to the chemokine family, members of which activate leukocytes and are often induced by proinflammatory stimuli such as lipopolysaccharide, TNF, or IL1. The chemokines are characterized by the presence of 4 conserved cysteines that form 2 disulfide bonds. They can be classified into 2 subfamilies. In the CC subfamily, the cysteine residues are adjacent to each other. In the CXC subfamily, they are separated by an intervening amino acid. The SDF1 proteins belong to the latter group.

CXCL12 is strongly chemotactic for lymphocytes. During embryogenesis it directs the migration of hematopoietic cells from foetal liver to bone marrow and the formation of large blood vessels. CXCL12 binds to its receptor CXCR4. LPS preferentially upregulates the expression of CXCR4 on CDS+ B-1 cells but not CD5− B-1 cells, not B-2 cells. Thus, activated CDS+ B-1 cells but not other B cells migrate towards CXCL12.

Recombinant CXCL12 protein can be obtained commercially, for example, from R&D Systems, Thermo Fisher Scientific, Novus Biologicals (product #P5357), Biolegend, and Peprotein. Alternative, standard molecular biology techniques can be used to express a recombinant CXCL12 protein. One skilled in the art would be to clone and synthesize recombinant protein. For example, as described in U.S. Pat. Nos. 7,923,016, 8,404,640, and 8,524,670, and U.S. Patent Application publication No: 20140045211, the contents are herein incorporated by reference in their entirety.

Chemokine (C—C motif) ligand 19 (CCL19) is a protein that in humans is encoded by the CCL19 gene. It plays a role in inflammatory and immunological responses, and also in normal lymphocyte recirculation and homing. For example, in trafficking of T-cells in thymus, and T-cell and B-cell migration to secondary lymphoid organs. CCL19 binds to the chemokine receptor CCR7. B-1 cells also express CCR7 and are attracted towards CCL19. Recombinant CCL19 has been shown to have potent chemotactic activity for T-cells and B-cells but not for granulocytes and monocytes.

Recombinant CCL19 protein can be obtained commercially, for example, from R&D Systems, Thermo Fisher Scientific, Novus Biologicals, Biolegend, Ebioscience, and MyBioSource. Alternative, standard molecular biology techniques can be used to express a recombinant CCL19 protein. One skilled in the art would be to clone and synthesize recombinant protein. For example, as described in U.S. Pat. Nos. 7,858,297, and 7,892,727, and U.S. Patent Application publication No: 20140045211, the contents are herein incorporated by reference in their entirety.

Granulocyte-macrophage colony-stimulating factor (GM-CSF), also known as colony stimulating factor 2 (CSF2), is a monomeric glycoprotein secreted by macrophages, T cells, mast cells, NK cells, endothelial cells and fibroblasts that functions as a cytokine. The pharmaceutical analogs of naturally occurring GM-CSF are called sargramostim and molgramostim.

GM-CSF is a monomeric glycoprotein that functions as a cytokine. It is a white blood cell growth factor. GM-CSF stimulates stem cells to produce granulocytes (neutrophils, eosinophils, and basophils) and monocytes. Monocytes exit the circulation and migrate into tissue, whereupon they mature into macrophages and dendritic cells. Thus, it is part of the immune/inflammatory cascade, by which activation of a small number of macrophages can rapidly lead to an increase in their numbers, a process crucial for fighting infection.

GM-CSF signals via signal transducer and activator of transcription, STATS. In macrophages, it has also been shown to signal via STAT3. The cytokine activates macrophages to inhibit fungal survival. It induces deprivation in intracellular free zinc and increases production of reactive oxygen species that culminate in fungal zinc starvation and toxicity. Thus, GM-CSF facilitates development of the immune system and promotes defense against infections. In mice, activated B-1 cells secrete GM-CSF and upregulate GM-CSF receptor as well. Autocrine stimulation by GM-CSF in B-1 cells promotes IgM production.

GM-CSF is manufactured using recombinant DNA technology and is marketed as a protein therapeutic called molgramostim or, when the protein is expressed in yeast cells, sargramostim (Amgen®). It is used as a medication to stimulate the production of white blood cells and thus prevent neutropenia following chemotherapy.

In one embodiment of any aspect described, the nanoparticle described herein comprises a chemokine selected from the group consisting of CXCL13, CXCL12 and CCL19, and GM-CSF.

In one embodiment of any aspect described, the nanoparticle described herein comprises only one chemokine selected from the group consisting of CXCL13, CXCL12 and CCL19.

In one embodiment of any aspect described, the nanoparticle described herein comprises only one chemokine selected from the group consisting of CXCL13, CXCL12 and CCL19, and GM-CSF.

In one embodiment of any aspect described, the nanoparticle described herein comprises only two chemokines, the two-chemokine combination is selected from the group consisting of CXCL13 and CXCL12; CXCL13 and CCL19; and CXCL12 and CCL19.

In one embodiment of any aspect described, the nanoparticle described herein comprises only two chemokines, the two-chemokine combination is selected from the group consisting of CXCL13 and CXCL12; CXCL13 and CCL19; and CXCL12 and CCL19; and GM-CSF.

In one embodiment of any aspect described, the nanoparticle described herein comprises all three chemokines CXCL13, CXCL12, and CCL19.

In one embodiment of any aspect described, the nanoparticle described herein comprises all three chemokines CXCL13, CXCL12, and CCL19; and GM-CSF.

In one embodiment of any aspect described, the nanoparticle described herein comprises one or more recombinantly produced chemokines selected from the group consisting of CXCL13, CXCL12 and CCL19.

In one embodiment of any aspect described, the nanoparticle described herein comprises one or more recombinantly produced chemokines selected from the group consisting of CXCL13, CXCL12 and CCL19; and GM-CSF.

In one embodiment of any aspect described, the nanoparticle described herein comprises one or more recombinantly produced human chemokines selected from the group consisting of CXCL13, CXCL12 and CCL19.

In one embodiment of any aspect described, the nanoparticle described herein comprises one or more recombinantly produced human chemokines selected from the group consisting of CXCL13, CXCL12 and CCL19; and GM-CSF.

### Thermosensitive Nanoparticles Carrying Chemokines

One embodiment of the present disclosure is to provide thermosensitive or pH-responsive nanoparticles that carrying chemokines, such as CXCL13, CXCL12 and CCL19, and compositions comprising these thermosensitive nanoparticles. These thermosensitive or pH-responsive nanoparticles are administered to a subject or directly placed at a target site in the subject, such as a tumor. Upon arrival in the tumor area, heat or change in pH may also be applied to trigger the release of the chemokines from within the nanoparticle. To achieve this, thermosensitive liposomes or pH-responsive are being developed. The formulation of such liposomes is based on pioneering work of Yatvin and Weinstein in the late 1970s (Yatvin M B et al., Science, 1978, 202:1290-3), who described the use of liposomes composed of phospholipids that undergo a gel-to-liquid crystalline phase transition at temperatures of around 44° C., a process which, in the absence of cholesterol in the membrane, causes significant release of liposome-entrapped water-soluble compounds.

From this basic formulation, thermosensitive liposomes have been further developed, for instance, by providing them with long-circulating properties using poly(ethylene glycol) (Needham D. et al., Cancer Res. 2000; 60:1197-201; Li L. et al., J. Control Release. 2010; 143:274-9; Unezaki S. et al., Pharm Res. 1994; 11:1180-5) or oligoglycerol-moieties (Lindner L H, et al., Clin Cancer Res. 2004; 10:2168-78) and by incorporating additional lipid compounds that further enhance membrane permeability at the phase transition temperature of the lipid membrane, e.g. lysolipid (Needham D. et al., Cancer Res. 2000; 60:1197-201) or oligoglycerol-PG (Lindner L H, et al., Clin Cancer Res. 2004; 10:2168-78).

Applicators for regional or localized heating of tumor tissue, even deep-seated tumors, are well established in clinical practice, and are used to heat tumor tissue to temperatures of 42° C. (mild hyperthermia). Mild hyperthermia involves the heating of tumors to temperatures of up to 43 degrees celcius and is usually combined with radiation or chemotherapy to enhance the therapeutic outcome of these treatments. Presently, hyperthermia technology has been perfected and can be applied locally on the tumor by focusing electromagnetic or ultrasound energy on the tumor area using, e.g., radiofrequency electrodes implanted in the tumor or microwave antennas and ultrasound transducers that apply their energy to the tumor non-invasively. Therefore, commonly used temperature-sensitive liposomal (TSL) are designed to release the encapsulated drug between 39° C. and 42° C.

Additional advancement in TSL has produced new thermal-sensitive liposome (the low temperature sensitive liposome (LTSL)) that responds at clinically attainable hyperthermic temperatures releasing its drug in a matter of seconds as it passes through the microvasculature of a warmed tumor. The LTSL is composed of a judicial combination of two or more component lipids, each with a specific function and each affecting specific material properties, including a sharp thermal transition and a rapid on-set of membrane permeability to small ions, drugs and small dextran polymers.

Conventional thermosensitive liposomes are composed of dipalmitoylphosphatidylcholine (DPPC), dipalmitoylphosphatidylglycerol (DPPG) or distearoyl phosphatidylcholine (DSPC). Phase transition temperatures of DPPC and DPPG are 41° C., while DSPC has the phase transition temperature of 58° C. Thermosensitive liposomes with a phase transition temperature of 42° C.-44° can be made by altering both type and molar ratio of lipids present in the bilayer of the liposomes. Apart from lipids, cholesterol and PEG are often used in thermosensitive liposomes. Cholesterol is a small steroid alcohol. It interacts with the hydrophobic tail of the phospholipids and hence stabilises the liposomal membrane. Addition of cholesterol lowers the phase transition temperature of liposomal membrane, but at the same time it broadens the phase transition temperature and thereby can modulate the temperature sensitivity of liposomes. So, optimum amount of cholesterol is necessary to prepare thermosensitive liposomes. A highly stable thermosensitive liposomal formulation consists primarily of DPPC, DSPC and cholesterol. Addition of PEG extends circulation time of liposomes, which is necessary for a better drug delivery system.

Besides addition of cholesterol, there are a number of strategies used to prepare thermosensitive liposomes, including addition of lysolipids along with other saturated lipids, grafting polymers with lipids, encapsulation of thermosensitive block copolymers, etc.

During mild hyperthermia, lysolipid-containing liposomes undergo major morphological changes like formation of open liposomes, bilayer disc and pore-like defects. For example, lysolipid-containing liposomes can completely release the encapsulated drug within 10-30 s at mild hyperthermia temperature, that is, 40° C.-42° C. During hyperthermia, encapsulated block copolymers disrupt the liposomal bilayer from inside and help in releasing liposomal contents quickly.

The liposomal membrane of the nanoparticles may show phase transition at the temperature of hyperthermia, i.e. so that the phase transition temperature of the membrane may be 39°-43° C. As the material of this membrane, various phospholipids of which acyl groups are saturated acyl groups (hereinafter sometimes abbreviated to “saturated phospholipids”) are used separately or in combination very advantageously. For example, glycerophospholipids are preferably used which have two acyl groups of the formula R—CO— wherein R is an alkyl group having 8 or more carbon atoms and at least one of the two R groups is an alkyl group having 10 or more, preferably 12-18, carbon atoms, and those of which the two alkyl groups have 12-18 carbon atoms each are preferably used. Such phospholipids include hydrogenated lecithin prepared by hydrogenation of lecithin originated from animals and plants (e.g. egg yolk lecithin, soybean lecithin), and phosphatidyl choline prepared by partial or totally-synthesis which contains mixed acyl groups of lauryl, myristoyl, palmitoyl, stearoyl, etc. Particularly phosphatidyl choline obtained by partial or total synthesis is used advantageously; the concrete examples used preferably are as follows (the observed phase transition temperatures are shown in parentheses): dimyristoylphosphatidyl choline (DMPC, 23.9° C.), palmitoylmyristoylphosphatidyl choline (PMPC, 27.2° C.), myristoylpalmitoylphosphatidyl choline (MPPC, 35.3° C.), dipalmitoylphosphatidyl choline (DPPC, 41.4° C.), stearoylpalmitoylphosphatidyl choline (SPPC, 44.0° C.), palmitoylstearoylphosphatidyl choline (PSPC, 47.4° C.), and distearoylphosphatidyl choline (DSPC, 54.9° C.).

The phase transition temperature of a liposomal membrane is approximate to the phase transition temperature calculated by weight-proportional distribution of those of individual saturated phospholipids used (See C. G. Knight, “Liposomes; from physical structure to therapeutic applications”, Elsevire, North Holland p. 310-311 (1981)), and the composition of saturated phospholipid can be chosen on the basis of this relationship so that the phase transition temperature of the membrane may be fall in the range described above. By adjustment of the phase transition temperature of the membrane to a temperature in the range described above and by adjustment of the osmotic pressure of the drug-containing solution to be entrapped which is described below, the object of this disclosure that the liposome compositions show phase transition of the membrane at the temperature of hyperthermia (38°-43° C.) so as to release effectively the drug entrapped can be achieved.

Low temperature-sensitive liposomal (LTSL) formulations are known in the art. One skilled in the art can be used prepare a themosensitive nanoparticle according to any method known in the art. For example, as described in U.S. Pat. Nos. 5,094,854, 6,726,925 and 7,901,709, and U.S. Patent Application Publication Nos; 2002/0102298, 2005/0191345, 2009/0087482, and 2009/0117035, and described in Akbarzadeh, A., et al., 2013, Nanoscale Res. Letts. 8:102; the contents of each patent or publication are incorporated herein by reference.

Accordingly, in one embodiment of any aspect described, the nanoparticle shell is permeable between the temperature 38° C. to 43° C.

In one embodiment of any aspect described, the nanoparticle shell is permeable below the pH of 7.0.

In one embodiment of any aspect described, the nanoparticle shell is a lipid layer.

In one embodiment of any aspect described, the nanoparticle shell comprises (a) one or more types of phospholipids selected from the group having two acyl groups, either saturated or unsaturated, and polar head group defined as phosphatidyl cholines, phosphatidyl glycerols, and/or phosphatidyl ethanolamines; (b) optionally a fatty acid or sterol with an ionizable moiety; and (c) one or more lysolipids selected from the group consisting of monoacylphosphatidyl cholines, monoacylphosphatidyl glycerols, and/or monoacylphosphatidyl ethanolomines.

Non-limiting examples of a fatty acid or sterol with an ionizable moiety include cholesterol-conjugated ionizable amino lipids or cholesterol with ionizable amine groups. For example, a lysine head group is a ionizable moiety and it can linked to a long-chain dialkylamine through an amide linkage at the lysine α-amine to an alcohol or a sterol.

In one embodiment of any aspect described, the lipid layer is a mixed lipid layer comprising two or more lipids.

In one embodiment of any aspect described, the mixed lipid layer comprises one or more types of phospholipids.

In one embodiment of any aspect described, the one or more types of phospholipids is/are selected from the group consisting of phosphatidyl cholines, phosphatidyl glycerols, phosphatidyl inositols and phosphatidyl ethanolamines.

In one embodiment of any aspect described, the phospholipid is selected from the group consisting of dipalmitoylphosphatidylcholine (DPPC), 1-palmitoyl-2-hydroxy-sn-glycero-3-phosphocholine (MPPC), 1-myristoyl-2-stearoyl-sn-glycero-3-phosphocholine (MSPC); 1,2-dimyristoyl-sn-glycero-3-phosphocholine (DMPC), 1,2-Dimyristoyl-sn-glycero-3-phosphorylglycerol (DMPG); 1,2-Distearoyl-sn-glycero-3-phosphoethanolamine (DSPE); 1,2-Dioleoyl-sn-glycero-3-phosphocholine (DOPC); 1,2-Dioleoyl-sn-glycero-3-phosphoethanolamine (DOPE); 1,2-dipalmitoyl-sn-glycero-3-phospho-(1′-rac-glycerol) (DPPG); 1,2-Distearoyl-sn-glycero-3-phosphocholine (DSPC); disteaoylphosphoethanolamine conjugated with polyethylene glycol (DSPE-PEG); phosphatidylserine (PS), phosphatidylethanolamine (PE), phosphatidylglycerol (PG), and phosphatidylcholine (PC).

In one embodiment of any aspect described, the one or more phospholipid is/are a lysolipid.

In one embodiment of any aspect described, the nanoparticle comprises a lysolipid-containing shell.

In one embodiment of any aspect described, the lysolipid is selected from the group consisting of monoacylphosphatidyl cholines, monoacylphosphatidyl glycerols, monoacylphosphatidyl inositols and/or monoacylphosphatidyl ethanolomines.

In one embodiment of any aspect described, the mixed lipid layer comprising of one or more of the lipid selected from the group consisting of DPPC, MPPC, PEG, DMPC, DMPG, DSPE, DOPC, DOPE, DPPG, DSPC, DSPE-PEG, MSPC, cholesterol, PS, PC, PE, and/or PG.

In one embodiment of any aspect described, the nanoparticle is a liposome comprising at least a first lipid bilayer. In one embodiment, the liposome comprises at least first lipid bilayer comprising of one or more of the lipid selected from the group consisting of DPPC, MPPC, PEG, DMPC, DMPG, DSPE, DOPC, DOPE, DPPG, DSPC, DSPE-PEG, MSPC, cholesterol, PS, PC, PE, and/or PG. In one embodiment, the nanoparticle is a liposome made of one or more of the lipid selected from the group consisting of DPPC, MPPC, PEG, DMPC, DMPG, DSPE, DOPC, DOPE, DPPG, DSPC, DSPE-PEG, MSPC, cholesterol, PS, PC, PE, and/or PG.

The size of the nanoparticles or liposomes in a preparation may depend upon the chemokine(s) entrapped contained therein and/or the intended target. Liposomes of between 0.05 to 0.3 microns in diameter, have been reported as suitable for tumor administration (U.S. Pat. No. 5,527,528 to Allen et al.). Sizing of nanoparticles or liposomes according to the present disclosure may be carried out according to methods known in the art, and taking into account the chemokine(s) contained therein and the effects desired (see, e.g., U.S. Pat. No. 5,225,212 to Martin et al; U.S. Pat. No. 5,527,528 to Allen et al., the disclosures of which are incorporated herein by reference in their entirety).

In one embodiment of any aspect described, the nanoparticle or liposome is less than 10 microns in diameter, or the nanoparticle or liposome preparation containing a plurality nanoparticles or liposomes respectively of less than 10 microns in diameter. In a further embodiment of any aspect described, nanoparticles or liposomes are from about 0.05 microns or about 0.1 microns in diameter, to about 0.3 microns or about 0.4 microns in diameter. The nanoparticle or iposome preparations may contain liposomes of different sizes. Advantageously, these nanoparticles or liposomes comprise lipid mixtures set forth herein and are therefore temperature-sensitive, with an ability to release contained chemokine(s), as described.

In one embodiment of any aspect described, the nanoparticles have a selected mean particle size of less than or equal to 150 nm.

In one embodiment of any aspect described, the nanoparticles have a selected mean particle size of between 60-150 nm.

In other embodiments of any aspect described, the nanoparticles have a selected mean particle size of between 65-150 nm, 70-150 nm, 75-150 nm, 80-150 nm, 85-150 nm, 90-150 nm, 95-150 nm, 100-150 nm, 105-150 nm, 110-150 nm, 115-150 nm, 120-150 nm, 125-150 nm, 130-150 nm, 135-150 nm, 140-150 nm, 60-145 nm, 60-140 nm, 60-135 nm, 60-130 nm, 60-125 nm, 60-120 nm, 60-115 nm, 60-110 nm, 60-105 nm, 60-100 nm, 60-95 nm, 60-90 nm, 60-85 nm, 60-80 nm, 60-75 nm, 60-70 nm, 70-145 nm, 70-140 nm, 70-135 nm, 70-130 nm, 70-125 nm, 70-120 nm, 70-115 nm, 70-110 nm, 70-105 nm, 70-100 nm, 70-95 nm, 70-90 nm, 70-85 nm, 70-80 nm, 80-145 nm, 80-140 nm, 80-135 nm, 80-130 nm, 80-125 nm, 80-120 nm, 80-115 nm, 80-110 nm, 80-105 nm, 80-100 nm, 80-95 nm, 80-90 nm, 90- 145 nm, 90-140 nm, 90-135 nm, 90-130 nm, 90-125 nm, 90-120 nm, 90-115 nm, 90-110 nm, 90-105 nm, 90-100 nm, 100-145 nm, 100-140 nm, 100-135 nm, 100-130 nm, 100-125 nm, 100-120 nm, 120-145 nm, 120-140 nm, 120-135 nm, 120-130 nm, and 130-145 nm.

In one embodiment of any aspect described, the nanoparticles have a selected mean particle size of about 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, or 145 nm, up to about 100 nm, 115 nm, 110 nm, 120 nm, 125 nm, 130 nm, 135 nm, 140 nm, 145 nm, or 150 nm in diameter.

In one embodiment of any aspect described, the nanoparticles or liposomes are prepared to have substantially homogeneous sizes in a selected size range. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane. See e.g., U.S. Pat. No. 4,737,323.

In one embodiment of any aspect described, the mixed lipid layer comprises of 5-20 mol % of MPPC or MSPC.

In one embodiment of any aspect described, the mixed lipid layer comprises of 5-18 mol % of MPPC or MSPC.

In one embodiment of any aspect described, the mixed lipid layer comprises of 8.5-10 mol % of MPPC or MSPC.

In one embodiment of any aspect described, the mixed lipid layer comprises of 85-95 mol % of DPPC or DPPG.

In one embodiment of any aspect described, the mixed lipid layer comprises of 0.1-10.0 mol % of DSPE-PEG.

In one embodiment of any aspect described, the mixed lipid layer comprises of no more that 4 mol % of DSPE-PEG.

In one embodiment of any aspect described, the mixed lipid layer is present below its phase transition temperature.

In one embodiment of any aspect described, the mixed lipid layer comprises DPPC and MSPC that are present in the molar ratio from about 95:5 to about 80:20.

In one embodiment of any aspect described, the mixed lipid layer comprises DPPC and MPPC that are present in the molar ratio from about 95:5 to about 80:20.

In one embodiment of any aspect described, the mixed lipid layer comprises DPPC and MSPC that are present in the molar ratio from about 95:5 to about 70:30.

In one embodiment of any aspect described, the mixed lipid layer comprises DPPC and MPPC that are present in the molar ratio from about 95:5 to about 70:30.

In one embodiment of any aspect described, the mixed lipid layer consists essentially of 5-20 mol % of MPPC or MSPC.

In one embodiment of any aspect described, the mixed lipid layer consists essentially of 5-18 mol % of MPPC or MSPC.

In one embodiment of any aspect described, the mixed lipid layer consists essentially of 8.5-10 mol % of MPPC or MSPC.

In one embodiment of any aspect described, the mixed lipid layer consists essentially of 85-95 mol % of DPPC or DPPG.

In one embodiment of any aspect described, the mixed lipid layer consists essentially of 0.1-10.0 mol % of DSPE-PEG.

In one embodiment of any aspect described, the mixed lipid layer consists essentially of no more that 4 mol % of DSPE-PEG.

In one embodiment of any aspect described, the DSPE-PEG is DSPE-PEG2000.

In one embodiment of any aspect described, the mixed lipid layer forms a lipid bilayer comprising of DPPC or DPPG; MPPC or MSPC; and DSPE-PEG.

In one embodiment of any aspect described, the mixed lipid layer forms a lipid bilayer comprising of DPPC, MPPC and DSPE-PEG.

In one embodiment of any aspect described, the mixed lipid layer forms a lipid bilayer comprising of DOPE, a fatty acid or sterol with an ionizable moiety, and DSPE-PEG.

In one embodiment of any aspect described, the mixed lipid layer forms a lipid bilayer comprising of DPPC, MPPC and DSPE-PEG in the molar ratio 90:10:4.

In one embodiment of any aspect described, the mixed lipid layer forms a lipid bilayer comprising of DPPC, MPPC and DSPE-PEG2000 in the molar ratio 86.5:7.3:3.8.

In one embodiment of any aspect described, the mixed lipid layer forms a lipid bilayer comprising of DPPC, MPPC and DSPE-PEG2000 in the molar ratio 85.0:9.8:5.2.

In one embodiment of any aspect described, the mixed lipid layer forms a lipid bilayer comprising of DPPC, MSPC and DSPE-PEG.

In one embodiment of any aspect described, the mixed lipid layer forms a lipid bilayer comprising of DPPC, MSPC and DSPE-PEG in the molar ratio 90:10:4.

In one embodiment of any aspect described, the mixed lipid layer forms a lipid bilayer comprising of DPPC, MSPC and DSPE-PEG2000 in the molar ratio 86.5:7.3:3.8.

In one embodiment of any aspect described, the mixed lipid layer forms a lipid bilayer comprising of DPPC, MSPC and DSPE-PEG2000 in the molar ratio 85.0:9.8:5.2.

In one embodiment of any aspect described, the nanoparticle comprises a second inner layer of mixed lipid which encapsulates the aqueous core comprising of the chemokine.

In one embodiment of any aspect described, the second layer of mixed lipid comprises of DPPC or DPPG; and MPPC or MSPC.

In one embodiment of any aspect described, the second layer of mixed lipid comprises of DPPC and MPPC.

In one embodiment of any aspect described, the second layer of mixed lipid comprises of DPPC and MPPC in the molar ratio 90:10.

In one embodiment of any aspect described, the second layer of mixed lipid comprises of DPPC and MPPC in the molar ratio 95:5 to about 80:20.

In one embodiment of any aspect described, the second layer of mixed lipid comprises of DPPC and MPPC in the molar ratio 95:5 to about 70:30.

In one embodiment of any aspect described, the second layer of mixed lipid comprises of DPPC and MSPC.

In one embodiment of any aspect described, the second layer of mixed lipid comprises of DPPC and MSPC in the molar ratio 90:10.

In one embodiment of any aspect described, the second layer of mixed lipid comprises of DPPC and MSPC in the molar ratio 95:5 to about 80:20.

In one embodiment of any aspect described, the second layer of mixed lipid comprises of DPPC and MSPC in the molar ratio 95:5 to about 70:30.

In one embodiment of any aspect described, the nanoparticle is a temperature-responsive liposome wherein the chemokine in the aqueous core is released from the nanoparticle when the environment of the nanoparticle is between 38° C. and 43° C.

In other embodiments of any aspect described, the chemokine in the aqueous core is released from the nanoparticle when the environment of the nanoparticle is between 39° C. and 43° C., between 39.5° C. and 43° C., between 40° C. and 43° C., between 40.5° C. and 43° C., between 41° C. and 43° C., between 41.5° C. and 43° C., between 42° C. and 43° C., between 38° C. and 42.5° C., between 38° C. and 42° C., between 38° C. and 41.5° C., between 38° C. and 41° C., between 38° C. and 40° C., between 39° C. and 42.5° C., between 39° C. and 42° C., between 39° C. and 41.5° C., between 39° C. and 41° C., between 39° C. and 40° C., between 39.5° C. and 42.5° C., between 39.5° C. and 42° C., between 39.5° C. and 41.5° C., between 40° C. and 42.5° C., between 40° C. and 42° C., between 40° C. and 41.5° C., and between 40° C. and 41° C.

In one embodiment of any aspect described, at least 70% of the chemokine therein is released when the environment of the nanoparticle experience mild hyperthermia. For example, when the temperature is between 38° C. and 43° C.

In other embodiments of any aspect described, at least 75%, at least 77%, at least 79%, at least 80%, at least 82%, at least 82%, at least 84%, at least 86%, at least 88%, at least 90%, at least 92%, at least 94%, at least 96%, at least 98%, at least 99%, at least 100% of the chemokine therein is released when the environment of the nanoparticle experience hyperthermia. For examples, the mild hyperthermia is between 39° C. and 43° C., between 39.5° C. and 43° C., between 40° C. and 43° C., between 40.5° C. and 43° C., between 41° C. and 43° C., between 41.5° C. and 43° C., between 42° C. and 43° C., between 38° C. and 42.5° C., between 38° C. and 42° C., between 38° C. and 41.5° C., between 38° C. and 41° C., between 38° C. and 40° C., between 39° C. and 42.5° C., between 39° C. and 42° C., between 39° C. and 41.5° C., between 39° C. and 41° C., between 39° C. and 40° C., between 39.5° C. and 42.5° C., between 39.5° C. and 42° C., between 39.5° C. and 41.5° C., between 40° C. and 42.5° C., between 40° C. and 42° C., between 40° C. and 41.5° C., and between 40° C. and 41° C.

In one embodiment of any aspect described, the chemokine is released within 5 minutes when the environment of the nanoparticle experience mild hyperthermia. For example, when the temperature is between 38° C. and 43° C.

In other embodiments of any aspect described, the chemokine is released within 4.9 min, 4.8 min, 4.7 min, 4.6 min, 4.5 min, 4.4 min, 4.3 min, 4.2 min, 4.1 min, 4.0 min, 3.9 min, 3.8 min, 3.7 min, 3.6 mins, 3.5 min, 3.4 min, 3.3 min, 3.2 min, 3.1 min, 3.0 minutes, 2.9 min, 2.8 min, 2.7 min, 2.6 min, 2.5 min, 2.4 min, 2.3 min, 2.2 min, 2.1 min, 2.0 min, 1.9 min, 1.8 min, 1.7 min, 1.6 min, 1.5 min, 1.4 min, 1.3 min, 1.2 min, 1.1 min, 1.0 min, 0.9 min, 0.8 min, 0.7 min, 0.6 min, and 0.5 min when the environment of the nanoparticle experience mild hyperthermia. For examples, the mild hyperthermia is between 39° C. and 43° C., between 39.5° C. and 43° C., between 40° C. and 43° C., between 40.5° C. and 43° C., between 41° C. and 43° C., between 41.5° C. and 43° C., between 42° C. and 43° C., between 38° C. and 42.5° C., between 38° C. and 42° C., between 38° C. and 41.5° C., between 38° C. and 41° C., between 38° C. and 40° C., between 39° C. and 42.5° C., between 39° C. and 42° C., between 39° C. and 41.5° C., between 39° C. and 41° C., between 39° C. and 40° C., between 39.5° C. and 42.5° C., between 39.5° C. and 42° C., between 39.5° C. and 41.5° C., between 40° C. and 42.5° C., between 40° C. and 42° C., between 40° C. and 41.5° C., and between 40° C. and 41° C.

In one embodiment of any aspect described, the nanoparticle is a temperature-responsive liposome wherein at least 70% of the chemokine in the aqueous core is released within 5 minutes when the environment of the nanoparticle is between 38° C. and 43° C.

Assessing the release of the nanoparticles or liposome contents can be performed by any method known in the art. For example, as described in Merlin, Eur. J. Cancer 27(8): 1031 (1979). The nanoparticles or liposome encapsulate fluorescent probe, 6-Carboxyfluorescein (CF) for the analysis. The CF was entrapped into liposomes at a quenching concentration (50 mM); no fluorescence was observed for CF entrapped in the liposome. Intense fluorescence, however, developed upon release of the probe from liposomes due to dilution of the CF in the suspension. The amount of the probe released from the liposomes at various temperatures could thus be quantified based on fluorescence. Merlin, Eur. J. Cancer., 27(8):1031 (1991), studied thermally sensitive liposomes encapsulating Doxorubicin (DX), and incorporated pegylated lipids in the bilayer to increase their circulation time in the blood stream compared to conventional thermosensitive liposomes. See also Maruyama et al., Biochem. Biophys. Acta, 1149:17 (1993)).

In one embodiment of any aspect described, the aqueous core comprises only one chemokine.

In one embodiment of any aspect described, the aqueous core comprises only two chemokines, the two-chemokine combination is selected from the group consisting of CXCL13 and CXCL12; CXCL13 and CCL19; and CXCL12 and CCL19. In one embodiment of any aspect described, the aqueous core further comprises GM-CSF.

In one embodiment of any aspect described, the aqueous core comprises all three chemokines CXCL13, CXCL12, and CCL19. In one embodiment of any aspect described, the aqueous core further comprises GM-CSF.

The amount of chemokine(s) to be entrapped within or carried by the nanoparticles or liposomes according to the present disclosure will vary depending on the therapeutic dose and the unit dose of the chemokine(s), as will be apparent to one skilled in the art. In general, however, the preparation of the nanoparticles or liposomes of the present disclosure is designed so the largest amount of chemokine(s) possible is carried by the the nanoparticle or liposome. The nanoparticles or liposomes of the present disclosule may be of any type.

In one embodiment of any aspect described, the aqueous core further comprises an inert molecule or compound for detection and/or imaging purposes. For example, detection of the location of the injected nanoparticle, or imaging the injected nanoparticle. Non-limiting examples of an inert molecule or compound for detection and/or imaging purposes include a fluorescent dye or radioactive dye or a heavy metal ion.

In one embodiment of any aspect described, the aqueous core further comprises a fluorescent dye or radioactive dye. For example, 6-Carboxyfluorescein (CF).

In one embodiment of any aspect described, the composition further comprises at least one pharmaceutically acceptable carrier, diluent, excipient or adjuvant.

In one embodiment of any aspect described, the composition further comprising a thermosensitive magnetic liposome (TSML).

In one embodiment of any aspect described, the TSML comprises magnetic fluid such as Fe3O4. To prepare magnetic liposomes, magnetic fluid Fe3O4 can be used as the core and co-encapsulated with ammonium sulfate buffer into the liposomes as described in Z. Peng et al., PLoS One. 2014; 9(3): e92924. Other non-limiting examples of TSML include those described in the U.S. Pat. No. 7,282,479 and U.S. Patent Application Publication Nos; 2011/0177153, 2005/0191345, and International Patent Application Publication No: WO2004071386; and methods described in A J. Giustinithe et al., Nano Life, 2010; 1(01n02), the contents of each patent or publication are incorporated herein by reference.

In one embodiment of any aspect described, the composition further comprises at least one cancer therapeutic agent. Non-limiting examples of least one cancer therapeutic agent includes gemcitabine, cisplastin, paclitaxel, carboplatin, bortezomib, AMG479, vorinostat, rituximab, temozolomide, rapamycin, ABT-737, PI-103; alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammaII and calicheamicin omegaII (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation.

In one embodiment of any aspect described, the nanoparticles are dispersed in physiological saline or PBS to provide an aqueous preparation of nanoparticles. Liposomes composed of DPPC:MPPC may be contained in physiological saline or PBS that contains from about 1 microMolar to about 5 microMolar of MPPC monomer.

### Therapeutic Use of Thermosensitive Nanoparticles Carrying Chemokines

Disclosed herein are methods of using the described nanoparticle for the treatment of cancer, especially, solid tumors. It is contemplated that the thermosensitive nanoparticles carrying chemokines can be used in every surgical procedure post tumor or tissue or organ resectioning. After removal of the tumor or diseased tissue or organ, the thermosensitive nanoparticles carrying chemokines can be applied directly to the site of resectioning. Locally increasing the temperature or decreasing the pH of the tissue at the excisinos site initiates the localized release of the chemokine(s) in the nanoparticles.

In one embodiment, this disclosure provides a method of treating cancer, the method comprising (a) administering a composition comprising a nanoparticle comprising at least a first lipid layer shell and an aqueous core to a subject's preselected tumor or cancer target site in need of treatment for cancer, wherein the aqueous core comprising at least one chemokine selected from the group consisting of CXCL13, CXCL12, and CCL19, wherein the at least first lipid layer shell encapsulates the aqueous core, and wherein the at least a lipid layer shell has a phase transition temperature between 38° C. and 43° C.; and (b) heating the subject's preselected tumor target site to a temperature of between 38° C. and 45° C., whereby the chemokine in the aqueous core is released from when the environment of the nanoparticle is between 38° C. and 43° C.

In one embodiment, this disclosure provides a method of treating cancer, the method comprising administering (a) a composition described herein to a subject in need of treatment for cancer; and (b) heating a subject's preselected tumor target site to a temperature of between 38° C. and 45° C., whereby the chemokine in the aqueous core is released from the nanoparticle when the environment of the nanoparticle is between 38° C. and 43° C.

In one embodiment, this disclosure provides a method of increasing infiltration of natural IgM producing B cells in a subject to the subject's tumor target site, the method comprising administering (a) a composition described herein to the subject; and (b) heating a subject's preselected tumor/cancer target site to a temperature of between 38° C. and 45° C., whereby the chemokine in the aqueous core of the nanoparticle of the composition is released from the nanoparticle when the environment of the nanoparticle is between 38° C. and 43° C.

In one embodiment of any aspect described, the treatment method comprises that the infiltration of natural IgM producing B cells to the tumor/cancer target site is increased by administration of the composition followed by the heating compared to prior to the heating.

In one embodiment of any aspect described, the treatment method comprises the released chemokine(s) at the tumor/cancer target site promotes in vivo infiltration of the subject's own innate natural IgM producing B cells to the tumor/cancer target site.

In one embodiment of any aspect described, the composition further comprises a themosensitive magnetic liposome (TSML).

In one embodiment of any aspect described, the treatment method further comprises administering a TSML or a magnetic liposome, or a second composition comprising a TSML or magnetic liposome.

In one embodiment of any aspect described, the TSML or magnetic liposome is administered concurrently with the composition described herein to the preselected target site.

In one embodiment of any aspect described, the TSML or magnetic liposome is administered sequentially prior to or after the administration of the composition described herein to the preselected target site. For example, the composition described herein is first injected to a preselected tumor target site, then a second composition comprising the TSML or magnetic liposome to the same preselected tumor target site.

The purpose of the TSML or magnetic liposome or the second composition comprising the TSML or magnetic liposome is to enable the mild hyperthermia to occur at the preselected tumor target site by changing the local magnetic field at and near the preselected tumor target site.

In one embodiment of any aspect described, the treatment method further comprises selecting a subject in need of cancer treatment or prevention.

In one embodiment of any aspect described, the subject in need of cancer treatment or prevention has been diagnosed with a cancer.

In one embodiment of any aspect described, the subject in need of cancer treatment or prevention has been diagnosed with a cancer and exhibits solid tumors in the body.

In one embodiment of any aspect described, the treatment method further comprises selecting a tumor target site for administering the nanoparticles described herein or compositions comprising the the nanoparticles described herein. For example, any tumors on the skin, or organs such as lung, muscle, liver, heart, spinal cord, brain, and kidney.

In one embodiment of any aspect described, the treatment method comprises that the preselected tumor target site is a solid tumor.

The nanoparticles described herein can be administered using methods that are known to those skilled in the art, including but not limited to delivery into the bloodstream of a subject or subcutaneous or intramuscular, or intracavity (peritneum, the lung, brain or liver etc) administration of liposomes. Where nanoparticles described herein are used in conjunction with hyperthermia, the nanoparticles can be administered by any suitable means that results in delivery of the nanoparticles to the treatment site. For example, nanoparticles can be administered intravenously and thereby brought to the site of a tumor by the normal blood flow; heating of this site can result in greater nanoparticles extravasation from the blood stream because of the effect of hyperthermnia on blood vasculature and moreover, once extravasated into the tumor tissue results in the nanoparticle or liposomal membranes being heated to the phase transition temperature so that the nanoparticle or liposomal contents are preferentially released at the site of the tumor.

For the treatment of cancer, effective delivery of the nanoparticles described via the bloodstream requires that the liposome be able to penetrate the continuous (but “leaky”) endothelial layer and underlying basement membrane surrounding the vessels supplying blood to a tumor. Liposomes of smaller sizes have been found to be more effective at extravasation into tumors through the endothelial cell barrier and underlying basement membrane which separates a capillary from tumor cells. See, e.g., U.S. Pat. No. 5,213,804.

As used herein, “solid tumors” are those growing in an anatomical site other than the bloodstream (in contrast to blood-borne tumors such as leukemias). Solid tumors require the formation of small blood vessels and capillaries to nourish the growing tumor tissue.

In accordance with the present disclosure, the anti-tumor or anti-neoplastic agent of choice is entrapped within a liposome according to the present disclosure; the liposomes are formulated to be of a size known to penetrate the endothelial and basement membrane barriers. The resulting liposomal formulation can be administered parenterally to a subject in need of such treatment, preferably by intravenous administration, but also by, for example, direct injection. Tumors characterized by an acute increase in permeability of the vasculature in the region of tumor growth are particularly suited for treatment by the present methods. Administration of liposomes is followed by heating of the treatment site to a temperature that results in release of the liposomal contents.

In one embodiment of any aspect described, the treatment method comprises the administration by direct intratumoral injection.

In one embodiment of any aspect described, the method of administration is by parenteral, oral, buccal, pulmonary, intravenous, intramuscular, subcutaneous, aural, rectal, vaginal, ophthalmic, intradermal, intraoccular, intracerebral, intralymphatic, intraarticular, intrathecal or intraperitoneal injection.

In one embodiment of any aspect described, the heating of step (b) in the treatment method is by high intensity focused ultrasound (HIFU) allows non-invasive heating to establish hyperthermia (of at least 40-45° C.) of the tumor/cancer target site over time.

In one embodiment of any aspect described, the heating of step (b) in the treatment method is by increasing the magnetic field around the vicinity of the tumor/cancer target site over time. In one embodiment, a high frequency magnetic field is preferably used, and a high frequency magnetic field with an electromagnetic wave having a frequency of 1 KHz to 10 MHz is particularly preferred. The reason why the high frequency magnetic field with a frequency higher than 1 KHz is preferred is that a heating efficiency due to magnetic hysteresis is high, and the reason why the high frequency magnetic field with a frequency lower than 10 MHz is preferred is that the magnetic fine particles can be heated while a heat generating reaction of a living thing due to induction current can be controlled.

Method of localized increase of the magnetic field in the body of an organism is known. For example, as described in the U.S. Patent Application Publication Nos; 2011/0177153, the contents of which is incorporated herein by reference.

In one embodiment of any aspect described, the subject is a mammal.

In one embodiment of any aspect described, the mammal is a primate mammal.

In one embodiment of any aspect described, the mammal is a human.

### Ex Vivo Expansion of Natural IgM Producing B Cells

The functional contributions of B-1 cells are intertwined in the tight regulation of their trafficking between different lymphoid compartments. B-1 cells migrate from the bone marrow and into the peritoneal cavity as they follow gradients of chemokines such as CXCL13, CXCL12 and CCL19. Therefore, the number of natural IgM-producing cells in circulation and available for cancer surveillance is limited and may not be sufficient when cancer growth occurs at a faster rate. Accordingly, the cancer therapy described herein uses a method of increasing the number of the natural IgM producing B cells ex vivo and adoptive transfer of the expanded cells into a subject for cancer treatment or for cancer prevention.

In one embodiment, this disclosure provides a method of expanding and/or stimulating natural IgM producing B cells derived from a subject, the method comprising culturing an isolated population of natural IgM producing B cell from a subject with a liposome comprising phosphatidylcholine (PC or PtC) and/or a composition comprising a liposome comprising PC for a period of time under culture conditions that promotes the expansion of the initial population of natural IgM producing B cells.

As used herein, the term “expanding” refers to increasing the number of like cells through cell division (mitosis). The term “proliferating” and “expanding” are used interchangeably.

The term “isolated” as used herein signifies that the cells are placed into conditions other than their natural environment. The term “isolated” does not preclude the later use of these cells thereafter in combinations or mixtures with other cells.

In one embodiment of any aspect described, the natural IgM-producing cells are phagocytic B cells.

In one embodiment of any aspect described, the natural IgM-producing cells are B-1 cells.

In one embodiment of any aspect described, the natural IgM-producing cells are phagocytic B-1 cells

In one embodiment of any aspect described, the natural IgM-producing cells are phagocytic L2pB1 cells.

In one embodiment of any aspect described, the natural IgM-producing cells not are phagocytic L2nB1 cells.

In one embodiment of any aspect described, the natural IgM-producing cells are constitutively producing IL-10.

In one embodiment of any aspect described, the natural IgM-producing cells are capable of self-renewal.

In one embodiment of any aspect described, the natural IgM-producing cells induce lipoptosis of cancer cells.

In one embodiment of any aspect described, the natural IgM-producing cells secrete GM-CSF.

In one embodiment of any aspect described, the natural IgM-producing cells are CD5⁺/CD27⁺ cells.

In one embodiment of any aspect described, the natural IgM-producing cells are CD69⁻/CD70⁻ cells.

In one embodiment of any aspect described, the natural IgM-producing cells are IgMhi secreting cells.

In one embodiment of any aspect described, the natural IgM-producing cells expresses CCR7, a chemokine receptor for CCL19.

In one embodiment of any aspect described, the natural IgM-producing cells are attracted towards CCL19.

In one embodiment of any aspect described, the natural IgM-producing cells are B220^{low}/CD5⁺/IgM^{hi}/CD11b⁺/PD-L2⁺.

In one embodiment of any aspect described, the natural IgM-producing cells are PtC-binding cells.

In one embodiment of any aspect described, the natural IgM-producing cells are CD5⁺/IgM^{hi}/CD27⁺/CD69⁻/CD70⁻/PtC-binding cells.

In one embodiment of any aspect described, the natural IgM-producing cells are CD5⁺/IgM^{hi}/CD27⁺/PtC-binding cells.

In one embodiment of any aspect described, the natural IgM-producing cells are CD20⁺/CD3⁻/IgM^{hi}/PD-L2⁺/CD27⁺/CD43^{+/-}/CD69⁻/CD70⁻.

In one embodiment of any aspect described, the natural IgM-producing cells are murine cells, porcine cells or human cells.

In one embodiment of any aspect described, the culturing is ex vivo.

In one embodiment of any aspect described, the cell expansion method further comprising selecting a subject who will donate the isolated natural IgM producing B cells.

In one embodiment of any aspect described, the cell expansion method further comprising collecting a sample of peritoneal cavity cells from the donor subject, wherein the sample comprises natural IgM producing B cells.

In one embodiment of any aspect described, the cell expansion method further comprising providing a sample of peritoneal cavity cells from the donor subject, wherein the sample comprises natural IgM producing B cells.

In one embodiment of any aspect described, the cell expansion method further comprising isolating a population of natural IgM producing B cell from the donor subject.

In one embodiment of any aspect described, the cell expansion method further comprising isolating a population of natural IgM producing B cell from a sample of peritoneal cavity cells from the donor subject.

In one embodiment of any aspect described, the cell expansion method further comprising selecting for natural IgM producing B cells from the subject prior to the ex vivo culturing. Murine natural IgM producing phagocytic B cells have the following markers: B220^{low}/CD5⁺/IgM^{hi}/CD11b⁺/PD-L2⁺. Human natural IgM producing phagocytic B cells have the following markers: CD20⁺/CD3⁻/IgM^{hi}/PD-L2⁺/CD27⁺/CD43^{+/-}CD69⁻CD70⁻. NIMPAB cells can be isolated by any method known in the industry. For example, non-limiting example include fluorescence-activated cell sorting based on the unique markers of the cells.

In one embodiment of any aspect described, the cell expansion method further comprising selecting for natural IgM producing B cells from the cell culture after the ex vivo culturing.

In one embodiment of any aspect described, the cell expansion method further comprising culturing the isolated population of NIMPAB cells with an infectious agent-derived ligands that bear the pathogen-associated molecular patterns (PAMPs), recognized by innate immune receptors, such as TLRs.

In one embodiment of any aspect described, the NIMPAB cells are cultured for at least 10 cell divisions. In other embodiment of any aspect described, the NIMPAB cells are cultured for about 5-7 cell divisions.

In one embodiment of any aspect described, the cell expansion method further comprising harvesting for the expanded natural IgM producing B cells from the cell culture after the ex vivo culturing and expansion. NIMPAB cells can be isolated by any method known in the industry. For example, non-limiting example include fluorescence-activated cell sorting based on the unique markers of the cells.

In one embodiment of any aspect described, the cell expansion method further comprising cryopreservation of the harvested natural IgM producing B cells prior to use.

In one embodiment, this disclosure provides a composition comprising a population of ex vivo culture expanded natural IgM producing B cells and a pharmaceutically acceptable carrier.

In one embodiment, this disclosure provides a composition comprising a population of ex vivo culture expanded natural IgM producing B cells and a pharmaceutically acceptable carrier for use in the treatment of cancer in a subject.

In another embodiment, this disclosure provides a composition comprising a population of ex vivo culture expanded natural IgM producing B cells and a pharmaceutically acceptable carrier for use in the manufacture of a medicament for the treatment of cancer in a subject.

In one embodiment, this disclosure provides a composition comprising a population of ex vivo culture expanded natural IgM producing B cells and a cryoprotective agent.

In one embodiment, this disclosure provides a composition comprising a population of ex vivo culture expanded natural IgM producing B cells and a cryoprotective agent for use in the treatment of cancer in a subject.

In another embodiment, this disclosure provides a composition comprising a population of ex vivo culture expanded natural IgM producing B cells and a cryoprotective agent for use in the manufacture of a medicament for the treatment of cancer in a subject.

As used herein, “composition” refers to an injectate, substance or a combination of substances which can be delivered into a tissue or an organ or a subject. Exemplary compositions include, but are not limited to, a suspension of ex vivo culture expanded natural IgM producing B cells described herein in a suitable physiologic carrier such as saline and/or with a cryoprotective agent.

In one embodiment of any aspect described, the composition further comprises an additional cancer therapeutic agent. Examples of cancer therapeutic agents are described herein.

In one embodiment of any aspect described, the composition further comprises a nanoparticle comprising a chemokine described herein. For example, a nanoparticle comprising a chemokine CXCL13 and/or CXCL12 and/or CCL19.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a subject without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present disclosure can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques. Suitable pharmaceutical carriers are described in Remington's Pharmaceutical Sciences, A. Osol, a standard reference text in this field of art. For example, a parenteral composition suitable for administration by injection is prepared by dissolving 1.5% by weight of active ingredient in 0.9% sodium chloride solution.

In one embodiment, the “pharmaceutically acceptable” carrier does not include in vitro cell culture media.

In one embodiment, the term “pharmaceutically acceptable” means approved by a regulatory agency of the Federal or a state government or listed in the U.S. Pharmacopeia or other generally recognized pharmacopeia for use in animals, and more particularly in humans. Specifically, it refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, and the like. Examples of suitable pharmaceutical carriers are described in Remington's Pharmaceutical Sciences, 18th Ed., Gennaro, ed. (Mack Publishing Co., 1990). The formulation should suit the mode of administration.

### Cryopreservation of Culture Expanded Natural IgM Producing B (NIMPAB) Cells

In one embodiment, the disclosure provides a cryopreserved composition comprising an enriched population of NIMPAB cells; an amount of cryopreservative sufficient for the cryopreservation of the isolated NIMPAB cells; and a pharmaceutically acceptable carrier. In one embodiment, the cryopreserved composition comprises a composition comprising an enriched population of NIMPAB cells; an amount of cryopreservative sufficient for the cryopreservation of the NIMPAB cells; and a pharmaceutically acceptable carrier.

Freezing is destructive to most living cells. Upon cooling, as the external medium freezes, cells equilibrate by losing water, thus increasing intracellular solute concentration. Below about 10°-15° C., intracellular freezing will occur. Both intracellular freezing and solution effects are responsible for cell injury (Mazur, P., 1970, Science 168:939-949). It has been proposed that freezing destruction from extracellular ice is essentially a plasma membrane injury resulting from osmotic dehydration of the cell (Meryman, H. T., et al., 1977, Cryobiology 14:287-302).

Cryoprotective agents and optimal cooling rates can protect against cell injury. Cryoprotection by solute addition is thought to occur by two potential mechanisms: colligatively, by penetration into the cell, reducing the amount of ice formed; or kinetically, by decreasing the rate of water flow out of the cell in response to a decreased vapor pressure of external ice (Meryman, H. T., et al., 1977, Cryobiology 14:287-302). Different optimal cooling rates have been described for different cells. Various groups have looked at the effect of cooling velocity or cryopreservatives upon the survival or transplantation efficiency of frozen bone marrow cells or red blood cells (Lovelock, J. E. and Bishop, M. W. H., 1959, Nature 183:1394-1395; Ashwood-Smith, M. J., 1961, Nature 190:1204-1205; RoWe, A. W. and Rinfret, A. P., 1962, Blood 20:636; Rowe, A. W. and Fellig, J., 1962, Fed. Proc. 21:157; Rowe, A. W., 1966, Cryobiology 3(1):12-18; Lewis, J. P., et al., 1967, Transfusion 7(1):17-32; Rapatz, G., et al., 1968, Cryobiology 5(1):18-25; Mazur, P., 1970, Science 168:939-949; Mazur, P., 1977, Cryobiology 14:251-272; Rowe, A. W. and Lenny, L. L., 1983, Cryobiology 20:717; Stiff, P. J., et al., 1983, Cryobiology 20:17-24; Gorin, N. C., 1986, Clinics in Haematology 15(1):19-48).

The successful recovery of human bone marrow cells after long-term storage in liquid nitrogen has been described (1983, American Type Culture Collection, Quarterly Newsletter 3(4):1). In addition, stem cells in bone marrow were shown capable of withstanding cryopreservation and thawing without significant cell death, as demonstrated by the ability to form equal numbers of mixed myeloid-erythroid colonies in vitro both before and after freezing (Fabian, I., et al., 1982, Exp. Hematol 10:119-122). The cryopreservation and thawing of human fetal liver cells (Zuckerman, A. J., et al., 1968, J. Clin. Pathol. (London) 21(1):109-110), fetal myocardial cells (Robinson, D. M. and Simpson, J. F., 1971, In Vitro 6(5):378), neonatal rat heart cells (Alink, G. M., et al., 1976, Cryobiology 13:295-304), and fetal rat pancreases (Kemp, J. A., et al., 1978, Transplantation 26(4):260-264) have also been reported.

The injurious effects associated with freezing can be circumvented by (a) use of a cryoprotective agent, (b) control of the freezing rate, and (c) storage at a temperature sufficiently low to minimize degradative reactions.

Cryoprotective agents which can be used include but are not limited to dimethyl sulfoxide (DMSO) (Lovelock, J. E. and Bishop, M. W. H., 1959, Nature 183:1394-1395; Ashwood-Smith, M. J., 1961, Nature 190:1204-1205), glycerol, polyvinylpyrrolidine (Rinfret, A. P., 1960, Ann. N.Y. Acad. Sci. 85:576), polyethylene glycol (Sloviter, H. A. and Ravdin, R. G., 1962, Nature 196:548), albumin, dextran, sucrose, ethylene glycol, i-erythritol, D-Sorbitol, D-mannitol (Rowe, A. W., et al., 1962, Fed. Proc. 21:157), D-sorbitol, i-inositol, D-lactose, choline chloride (Bender, M. A., et al., 1960, J. Appl. Physiol. 15:520), amino acids (Phan The Tran and Bender, M. A., 1960, Exp. Cell Res. 20:651), methanol, acetamide, glycerol monoacetate (Lovelock, J. E., 1954, Biochem. J. 56:265), and inorganic salts (Phan The Tran and Bender, M. A., 1960, Proc. Soc. Exp. Biol. Med. 104:388; Phan The Tran and Bender, M. A., 1961, in Radiobiology, Proceedings of the Third Australian Conference on Radiobiology, Ilbery, P. L. T., ed., Butterworth, London, p. 59). In a preferred embodiment, DMSO is used, a liquid which is non-toxic to cells in low concentration. Being a small molecule, DMSO freely permeates the cell and protects intracellular organelles by combining with water to modify its freezability and prevent damage from ice formation. Addition of plasma (e.g., to a concentration of 20-25%) can augment the protective effect of DMSO. After addition of DMSO, cells should be kept at 0-4° C. until freezing, since DMSO concentrations of about 1% are toxic at temperatures above 4° C.

A controlled slow cooling rate is critical. Different cryoprotective agents (Rapatz, G., et al., 1968, Cryobiology 5(1):18-25) and different cell types have different optimal cooling rates (see e.g., Rowe, A. W. and Rinfret, A. P., 1962, Blood 20:636; Rowe, A. W., 1966, Cryobiology 3(1):12-18; Lewis, J. P., et al., 1967, Transfusion 7(1):17-32; and Mazur, P., 1970, Science 168:939-949 for effects of cooling velocity on survival of marrow-stem cells and on their transplantation potential). The heat of fusion phase where water turns to ice should be minimal. The cooling procedure can be carried out by use of, e.g., a programmable freezing device or a methanol bath procedure.

Programmable freezing apparatuses allow determination of optimal cooling rates and facilitate standard reproducible cooling. Programmable controlled-rate freezers such as Cryomed or Planar permit tuning of the freezing regimen to the desired cooling rate curve. For example, for marrow cells in 10% DMSO and 20% plasma, the optimal rate is 1 to 3° C./minute from 0° C. to −80° C. The container holding the cells must be stable at cryogenic temperatures and allow for rapid heat transfer for effective control of both freezing and thawing. Sealed plastic vials (e.g., Nunc, Wheaton Cryules®) or glass ampules can be used for multiple small amounts (1-2 ml), while larger volumes (100-200 ml) can be frozen in polyolefin bags (e.g., Delmed) held between metal plates for better heat transfer during cooling. (Bags of bone marrow cells have been successfully frozen by placing them in −80° C. freezers which, fortuitously, gives a cooling rate of approximately 3° C./minute).

In an alternative embodiment, the methanol bath method of cooling can be used. The methanol bath method is well-suited to routine cryopreservation of multiple small items on a large scale. The method does not require manual control of the freezing rate nor a recorder to monitor the rate. In a preferred aspect, DMSO-treated cells are pre-cooled on ice and transferred to a tray containing chilled methanol which is placed, in turn, in a mechanical refrigerator (e.g., Harris or Revco) at −80° C. Thermocouple measurements of the methanol bath and the samples indicate the desired cooling rate of 1° to 3° C./minute. After at least two hours, the specimens have reached a temperature of −80° C. and can be placed directly into liquid nitrogen (−196° C.) for permanent storage.

After thorough freezing, cells can be rapidly transferred to a long-term cryogenic storage vessel. Such storage is greatly facilitated by the availability of highly efficient liquid nitrogen refrigerators, which resemble large Thermos containers with an extremely low vacuum and internal super insulation, such that heat leakage and nitrogen losses are kept to an absolute minimum

In one embodiment, the cryopreservation procedure described in Current Protocols in Stem Cell Biology, 2007, (Mick Bhatia, et. al., ed., John Wiley and Sons, Inc.) is used for the compositions of isolated and expanded cells described herein. The reference is hereby incorporated by reference. As as exemplary, when the NIMPAB cells on a 10-cm tissue culture plate have reached at least 50% confluency, preferably 70% confluency, the media within the plate is aspirated and the cells are rinsed with phosphate buffered saline. The adherent cells are then detached by 3 ml of 0.025% trypsin/0.04% EDTA treatment. The trypsin/EDTA is neutralized by 7 ml of media and the detached cells are collected by centrifugation at 200×g for 2 min. The supernatant is aspirated off and the pellet of cells is resuspended in 1.5 ml of media. The harvested NIMPAB cells are cryopreserved at a density of at least 3×103 cells/ml. A aliquot of 1 ml of 100% DMSO is added to the suspension of NIMPAB cells and gently mixed. Then 1 ml aliquot of this suspension of NIMPAB cells in DMSO is dispensed into cyrules in preparation for cryopreservation. The sterilized storage cryules preferably have their caps threaded inside, allowing easy handling without contamination. Suitable racking systems are commercially available and can be used for cataloguing, storage, and retrieval of individual specimens.

Other methods of cryopreservation of viable cells, or modifications thereof, are available and envisioned for use (e.g., cold metal-mirror techniques; Livesey, S. A. and Linner, J. G., 1987, Nature 327:255; Linner, J. G., et al., 1986, J. Histochem. Cytochem. 34(9):1123-1135; U.S. Pat. Nos. 4,199,022, 3,753,357, 4,559,298 and are incorporated hereby reference.

Recovering NIMPAB Cells from the Frozen State

When the NIMPAB cells are needed, the frozen NIMPAB cells can be thawed according to methods known in the art, and used in the therapeutic methods described herein.

Frozen NIMPAB cells are preferably thawed quickly (e.g., in a water bath maintained at 37°-41° C.) and chilled on ice immediately upon thawing. In particular, the cryogenic vial containing the frozen NIMPAB cells can be immersed up to its neck in a warm water bath; gentle rotation will ensure mixing of the cell suspension as it thaws and increase heat transfer from the warm water to the internal ice mass. As soon as the ice has completely melted, the vial can be immediately placed in ice.

In a particular embodiment, the thawing procedure after cryopreservation is described in Current Protocols in Stem Cell Biology 2007 (Mick Bhatia, et. al., ed., John Wiley and Sons, Inc.) and is hereby incorporated by reference. Immediately after removing the cryogenic vial from the cryo-freezer, the vial is rolled between the hands for 10 to 30 sec until the outside of the vial is frost free. The vial is then held upright in a 37° C. water-bath until the contents are visibly thawed. The vial is immersed in 95% ethanol or sprayed with 70% ethanol to kill microorganisms from the water-bath and air dry in a sterile hood. The contents of the vial are then transferred to a 10-cm sterile culture containing 9 ml of media using sterile techniques. The NIMPAB cells can then be cultured and further expanded in a incubator at 37° C. with 5% humidified CO2.

It may be desirable to treat the NIMPAB cells in order to prevent cellular clumping upon thawing. To prevent clumping, various procedures can be used, including but not limited to, the addition before and/or after freezing of DNase (Spitzer, G., et al., 1980, Cancer 45:3075-3085), low molecular weight dextran and citrate, hydroxyethyl starch (Stiff, P. J., et al., 1983, Cryobiology 20:17-24).

The cryoprotective agent, if toxic in humans, should be removed prior to therapeutic use of the thawed NIMPAB cells. In an embodiment employing DMSO as the cryopreservative, it is preferable to omit this step in order to avoid cell loss, since DMSO has no serious toxicity. However, where removal of the cryoprotective agent is desired, the removal is preferably accomplished upon thawing.

One way in which to remove the cryoprotective agent is by dilution to an insignificant concentration. This can be accomplished by addition of medium, followed by, if necessary, one or more cycles of centrifugation to pellet the cells, removal of the supernatant, and resuspension of the cells. For example, the intracellular DMSO in the thawed cells can be reduced to a level (less than 1%) that will not adversely affect the recovered cells. This is preferably done slowly to minimize potentially damaging osmotic gradients that occur during DMSO removal.

After removal of the cryoprotective agent, cell count (e.g., by use of a hemocytometer) and viability testing (e.g., by trypan blue exclusion; Kuchler, R. J. 1977, Biochemical Methods in Cell Culture and Virology, Dowden, Hutchinson & Ross, Stroudsburg, Pa., pp. 18-19; 1964, Methods in Medical Research, Eisen, H. N., et al., eds., Vol. 10, Year Book Medical Publishers, Inc., Chicago, pp. 39-47) can be done to confirm cell survival.

Other procedures which can be used, relating to processing of the thawed cells, include enrichment for adherent NIMPAB cells and expansion by in vitro culture as described supra.

In one embodiment of any aspect of this disclosure, thawed NIMPAB cells are tested by standard assays of viability (e.g., trypan blue exclusion) and of microbial sterility as described herein, and tested to confirm and/or determine their identity relative to the recipient.

Endotoxin levels can be determined by the gel-clot limulus amebocyte lysate (LAL) test method in compliance with the US Food and Drug Administration's GMP regulations, 21 CFR § 211. Acceptable endotoxin level is 5.0 EU/ml.

An aliquot of the cells will be taken prior to cryopreservation for mycoplasma PCR testing. The Mycoplasma PCR testing will be performed at a GMP approved facility using MycoSensor™ QPCR Assay Kit (Manufactured by Stratagene).

Methods for identity testing which can be used include but are not limited to HLA typing (Bodmer, W., 1973, in Manual of Tissue Typing Techniques, Ray, J. G., et al., eds., DHEW Publication No. (NIH) 74-545, pp. 24-27), and DNA fingerprinting, which can be used to establish the genetic identity of the cells. DNA fingerprinting (Jeffreys, A. J., et al., 1985, Nature 314:67-73) exploits the extensive restriction fragment length polymorphism associated with hypervariable minisatellite regions of human DNA, to enable identification of the origin of a DNA sample, specific to each individual (Jeffreys, A. J., et al., 1985, Nature 316:76; Gill, P., et al., 1985, Nature 318:577; Vassart, G., et al., 1987, Science 235:683), and is thus preferred for use.

### Therapeutic Uses of the Ex Vivo Expanded of Natural IgM Producing B Cells

In one embodiment, this disclosure provides a method of treating cancer, the method comprising administering a population of ex vivo culture expanded natural IgM producing B cells to a subject in need of treatment for cancer, wherein the natural IgM producing B cells are culture expanded by any method described. It is contemplated that the population of ex vivo culture expanded natural IgM producing B cells or a composition comprising the population of ex vivo culture expanded natural IgM producing B cells can be used in every surgical procedure post tumor or tissue or organ re-sectioning. After removal of the tumor or diseased tissue or organ, the a population of ex vivo culture expanded natural IgM producing B cells or a composition comprising the population of ex vivo culture expanded natural IgM producing B cells can be applied directly to the site of re-sectioning or systemically to the subject. It is contemplated that the population of ex vivo culture expanded natural IgM producing B cells or a composition comprising the population of ex vivo culture expanded natural IgM producing B cells be used routinely in any cancer therapy, for treatment and also for prevention after the subject has gone into remission.

In one embodiment, this disclosure provides a method of treating cancer in a subject in need of cancer treatment, the method comprising (a) culturing an initial population of natural IgM producing B cell with a liposome comprising phosphatidylcholine (PC) and/or a composition comprising a liposome comprising PC for a period of time under culture conditions that promotes the expansion of the initial population of natural IgM producing B cells; (b) culturing the cell ex vivo; and (c) administering a therapeutically effective amount of the expanded cell to a recipient subject in need of treatment for cancer.

As used herein, the term “amount” refers to “an amount effective” or “an effective amount” of NIMPAB cells to achieve a beneficial or desired prophylactic or therapeutic result, including clinical results. For example, cause apoptosis of the cancer cells, shrink the size of the tumor and/or reduce the rate of tumor growth.

A “prophylactically effective amount” refers to an amount of NIMPAB cells to achieve the desired prophylactic result. For example, to prevent the relapse of tumor growth after complete removal or shrinkage of tumor. Typically but not necessarily, since a prophylactic dose is used in subjects prior to or at an earlier stage of disease, the prophylactically effective amount is less than the therapeutically effective amount.

A “therapeutically effective amount” of NIMPAB cells may vary according to factors such as the cancer stage, age, sex, health and weight of the individual, and the ability of the NIMPAB cells to elicit a desired response in the individual. A therapeutically effective amount is also one in which any toxic or detrimental effects of NIMPAB cells are outweighed by the therapeutically beneficial effects. The term “therapeutically effective amount” includes an amount that is effective to “treat” a subject (e.g., a patient).

In one embodiment, as used herein, “prevent,” and similar words such as “prevented,” “preventing” etc., indicate an approach for preventing, inhibiting, or reducing the likelihood of the occurrence or recurrence of cancer and tumor growth. In another embodiment, the term refers to delaying the onset or recurrence of cancer and tumor growth, or delaying the occurrence or recurrence of the symptoms associated with cancer and tumor growth. In another embodiment, as used herein, “prevention” and similar words includes reducing the intensity, effect, symptoms and/or burden of cancer and tumor growth prior to onset or recurrence of cancer and tumor growth.

In one embodiment, as used herein “treatment” or “treating,” includes any beneficial or desirable effect on the symptoms or pathology of cancer and tumor growth, and may include even minimal reductions in one or more measurable markers of the cancer and tumor growth being treated. In another embodiment, treatment can involve optionally either the reduction or amelioration of symptoms of cancer and tumor growth, or the delaying of the progression of the cancer and tumor growth. “Treatment” does not necessarily indicate complete eradication or cure of the cancer and tumor growth, or associated symptoms thereof.

In one embodiment, when used herein in reference to cells, the term “administering,” refers to the placement of the natural IgM producing B cells into the recipient subject. In one embodiment, “administering,” refers to the cells being placed directily into a tumor or near a tumor in the recipient subject. In other embodiments, “administering,” refers to the placing the cells intravenously into the recipient subject.

In another embodiment, when used herein in reference to nanoparticles described herein, liposomes described herein, or compositions comprising the nanoparticles or liposomes described herein, the term “administering,” refers to the placement of the nanoparticles, or liposomes, or compositions into the recipient subject. In one embodiment, “administering,” refers to the nanoparticles, or liposomes, or compositions being placed directily into a tumor or near a tumor in the recipient subject. In other embodiments, “administering,” refers to the placing the nanoparticles, or liposomes, or compositions intravenously into the recipient subject.

In one embodiment of any aspect described, the cell treatment method further comprising selecting a subject who will donate the isolated natural IgM producing B cells.

In one embodiment of any aspect described, the donor subject is a healthy subject who has not been diagnosed with cancer.

In one embodiment of any aspect described, the donor subject is a subject who has been diagnosed with cancer.

In one embodiment of any aspect described, the cell treatment method further comprising selecting a receipient subject who will be administered the expanded natural IgM producing B cells.

In one embodiment of any aspect described, the recipient subject has cancer.

In one embodiment of any aspect described, the cell treatment method further comprising collecting a sample of peritoneal cavity cells from the donor subject, wherein the sample comprises natural IgM producing B cells.

In one embodiment of any aspect described, the treatment method further comprising providing a sample of peritoneal cavity cells from the donor subject, wherein the sample comprises natural IgM producing B cells.

In one embodiment of any aspect described, the treatment method further comprising a step of selecting for the expanded natural IgM-producing cells prior to administering the cells to the recipient subject.

In one embodiment of any aspect described, the treatment method further comprising a step of harvesting the expanded natural IgM-producing cells prior to administering the cells to the recipient subject.

In one embodiment of any aspect described, the treatment method further comprising a step of enriching for expanded natural IgM-producing cells prior to administering the cells to the recipient subject.

In one embodiment of any aspect described, the treatment method further comprising a step of cryopreserving the expanded natural IgM-producing cells prior to administering the cells to the recipient subject.

In one embodiment of any aspect of the treatment method described, the natural IgM-producing cells are phagocytic B cells.

In one embodiment of any aspect of the treatment method described, the natural IgM-producing cells are B-1 cells.

In one embodiment of any aspect of the treatment method described, the natural IgM-producing cells are phagocytic B-1 cells

In one embodiment of any aspect of the treatment method described, the natural IgM-producing cells are phagocytic L2pB1 cells.

In one embodiment of any aspect described, the natural IgM-producing cells are B220low/CD5+/IgMhi/CD11b+/PD-L2+.

In one embodiment of any aspect described, the natural IgM-producing cells are CD20+/CD3−/IgMhi/PD-L2+/CD27+/CD43+/−CD69−/CD70−.

In one embodiment of any aspect described, the natural IgM-producing cells are murine cells, porcine cells or human cells.

In one embodiment of any aspect described, the culturing is ex vivo.

In one embodiment of any aspect of the treatment method described, the natural IgM-producing cell is obtained from a healthy donor subject.

In one embodiment of any aspect of the treatment method described, the natural IgM-producing cell is obtained from peripheral blood; through hemodialysis; from the peritoneal cavity; through peritoneal dialysis; or from a tumor sample.

In one embodiment of any aspect of the treatment method described, the donor subject and the recipient subject are not the same subject.

In one embodiment of any aspect of the treatment method described, the natural IgM-producing cell is non-autologous to the recipient subject.

In one embodiment of any aspect of the treatment method described, the non-autologous natural IgM-producing cell is at the minimum HLA match with the recipient subject.

The natural IgM producing B cells isolated from a donor subject is an HLA-type match with a host (recipient) subject who is diagnosed with cancer or at risk of developing relapse of cancer. Donor-recipient antigen type-matching is well known in the art. The HLA-types include HLA-A, HLA-B, HLA-C, and HLA-D. These represent the minimum number of cell surface antigen matching required for adoptive transfer or transplantation of non-autologous cells. That is the transfected cells are transplanted into a different subject, i.e., allogeneic to the recipient host subject.

In one embodiment of any aspect of the treatment method described, the donor subject and the recipient subject are the same subject, i.e., the natural IgM producing B cells isolated from are autologous to the recipient subject.

In one embodiment of any aspect of the treatment method described, the natural IgM-producing cell is autologous to the recipient subject.

In one embodiment of any aspect of the treatment method described, the administration is by direct intratumoral injection.

In one embodiment of any aspect of the treatment method described, the method of administration is by parenteral, oral, buccal, pulmonary, intravenous, intramuscular, subcutaneous, aural, rectal, vaginal, ophthalmic, intradermal, intraoccular, intracerebral, intralymphatic, intraarticular, intrathecal or intraperitoneal injection.

In one embodiment of any aspect of the treatment method described, the subject is a mammal.

In one embodiment of any aspect of the treatment method described, the mammal subject is a primate mammal.

In one embodiment of any aspect of the treatment method described, the mammal is a human.

In one embodiment of any aspect described, a “subject,” as used herein, includes any animal that exhibits a symptom of a cancer that can be treated with the natural IgM producing B cells, and methods disclosed elsewhere herein.

The NIMPAB cells or the compositions comprising the NIMPAB cells described herein can be administered by any known route that would achieve the objective of placement of the cells into a tumor or in the vinicity a tumor in a subject. For example, the NIMPAB cells or the compositions comprising the NIMPAB cells described herein are administered via intravenously or by intratumoral injection.

In addition, the NIMPAB cells herein can be administered together with other components of biologically active agents, such as pharmaceutically acceptable surfactants (e.g., glycerides), excipients (e.g., lactose), carriers, diluents and vehicles.

The dosage administered to a subject will vary depending upon a variety of factors, including the size of the tumor and stage of cancer of the subject, and the route of administration; size, age, sex, health, body weight and diet of the recipient subject. For example, a dose can be about 1000 to 1 million NIMPAB cells per dose. For example, a larger tumor size may require a larger dose of cells administered. The larger tumor would require several intratumoral injections at several locations of the tumor. For example, an intratumoral injection per one cubic centimeter.

In one embodiment of any aspect described, about 1000 to 1 million NIMPAB cells per dose are administered to the recipient subject.

In another embodiment of any aspect described, at least 10 million NIMPAB cells per dose are administered to the recipient subject.

In another embodiment of any aspect described, about 10⁶ NIMPAB cells, about 10⁷ NIMPAB cells, about 10⁸ NIMPAB cells, about 10⁹ NIMPAB cells, about 10¹⁰ NIMPAB cells, or about 10¹¹ NIMPAB cells are administered to the recipient subject.

In one embodiment of any aspect described, the recipient subject receives only one dose of NIMPAB cells.

In another embodiment of any aspect described, the recipient subject receives more than one dose of NIMPAB cells. For example, one intratumoral injection per week over a period of 2-3 months.

In other embodiments, the recipient subject receives a dose of natural IgM producing B cells, of about 1×10⁵ cells/kg, about 5×10⁵ cells/kg, about 1×10⁶ cells/kg, about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 5×10⁷ cells/kg, about 1×10⁸ cells/kg, or more in one single intravenous or injection dose. In certain embodiments, the recipient subject receives a dose of natural IgM producing B cells, of at least 1×10⁵ cells/kg, at least 5×10⁵ cells/kg, at least 1×10⁶ cells/kg, at least 2×10⁶ cells/kg, at least 3×10⁶ cells/kg, at least 4×10⁶ cells/kg, at least 5×10⁶ cells/kg, at least 6×10⁶ cells/kg, at least 7×10⁶ cells/kg, at least 8×10⁶ cells/kg, at least 9×10⁶ cells/kg, at least 1×10⁷ cells/kg, at least 5×10⁷ cells/kg, at least 1×10⁸ cells/kg, or more in one single intravenous or injection dose.

In an additional embodiment, the recipient subject receives a dose of ex vivo expanded natural IgM producing B cells of about 1×10⁵ cells/kg to about 1×10⁸ cells/kg, about 1×10⁶ cells/kg to about 1×10⁸ cells/kg, about 1×10⁶ cells/kg to about 9×10⁶ cells/kg, about 2×10⁶ cells/kg to about 8×10⁶ cells/kg, about 2×10⁶ cells/kg to about 8×10⁶ cells/kg, about 2×10⁶ cells/kg to about 5×10⁶ cells/kg, about 3×10⁶ cells/kg to about 5×10⁶ cells/kg, about 3×10⁶ cells/kg to about 4×10⁸ cells/kg, or any intervening dose of cells/kg.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

It should be understood that the preceding is merely a detailed description of certain embodiments. It therefore should be apparent to those of ordinary skill in the art that various modifications and equivalents can be made without departing from the spirit and scope of the disclosure, and with no more than routine experimentation.

All references, patents and patent applications that are recited in this application are incorporated by reference herein in their entirety.

The present invention can be defined in any of the following numbered paragraphs:
[1] A nanoparticle comprising at least a lipid layer shell and an aqueous core, wherein the aqueous core comprising at least one chemokine selected from the group consisting of CXCL13, CXCL12, and CCL19, wherein the at least a lipid layer shell encapsulates the aqueous core, and wherein the at least a lipid layer shell has a phase transition temperature between 38° C. and 43° C.
[2] The nanoparticle of paragraph 1, wherein the lipid layer is a mixed lipid layer comprising two or more lipids.
[3] The nanoparticle of paragraph 2, wherein the mixed lipid layer comprises one or more phospholipids.
[4] The nanoparticle of paragraph 3, wherein the one or more phospholipids is/are selected from the group consisting of phosphatidyl cholines, phosphatidyl glycerols, phosphatidyl inositols and phosphatidyl ethanolamines.
[5] The nanoparticle of any one of paragraphs 3-5, wherein the phospholipid is selected from the group consisting of dipalmitoylphosphatidylcholine (DPPC), 1-palmitoyl-2-hydroxy-sn-glycero-3-phosphocholine (MPPC), 1-myristoyl-2-stearoyl-sn-glycero-3-phosphocholine (MSPC); 1,2-dimyristoyl-sn-glycero-3-phosphocholine (DMPC), 1,2-Dimyristoyl-sn-glycero-3-phosphorylglycerol (DMPG); 1,2-Distearoyl-sn-glycero-3-phosphoethanolamine (DSPE); 1,2-Dioleoyl-sn-glycero-3-phosphocholine (DOPC); 1,2-Dioleoyl-sn-glycero-3-phosphoethanolamine (DOPE); 1,2-dipalmitoyl-sn-glycero-3-phospho-(1′-rac-glycerol) (DPPG); 1,2-Distearoyl-sn-glycero-3-phosphocholine (DSPC); disteaoylphosphoethanolamine conjugated with polyethylene glycol (DSPE-PEG); phosphatidylserine (PS), phosphatidylethanolamine (PE), phosphatidylglycerol (PG), and phosphatidylcholine (PC).
[6] The nanoparticle of paragraphs 4-5, wherein the one or more phospholipid is/are a lysolipid.
[7] The nanoparticle of paragraph 6, wherein the lysolipid is selected from the group consisting of monoacylphosphatidyl cholines, monoacylphosphatidyl glycerols, monoacylphosphatidyl inositols and/or monoacylphosphatidyl ethanolomines.
[8] The nanoparticle of any one of paragraphs 2-7, wherein the mixed lipid layer comprising of one or more of the lipid selected from the group consisting of DPPC, MPPC, PEG, DMPC, DMPG, DSPE, DOPC, DOPE, DPPG, DSPC, DSPE-PEG, MSPC, cholesterol, PS, PC, PE, and/or PG,
[9] The nanoparticle of any one of paragraphs 1-8, wherein the nanoparticle is a liposome comprising at least a lipid bilayer.
[10] The nanoparticle of any one of paragraphs 1-9, wherein the nanoparticles have a selected mean particle size of less than or equal to 150 nm.
[11] The nanoparticle of any one of paragraphs 1-10, wherein the nanoparticles have a selected mean particle size of between 60-150 nm.
[12] The nanoparticle of any one of paragraphs 2-11, wherein the mixed lipid layer comprises 5-20 mol % of MPPC or MSPC.
[13] The nanoparticle of any one of paragraphs 2-12, wherein the mixed lipid layer comprises 5-18 mol % of MPPC or MSPC.
[14] The nanoparticle of any one of paragraphs 2-13, wherein the mixed lipid layer comprises 8.5-10 mol % of MPPC or MSPC.
[15] The nanoparticle of any one of paragraphs 2-14, wherein the mixed lipid layer comprises 85-95 mol % of DPPC or DPPG.
[16] The nanoparticle of any one of paragraphs 2-15, wherein the mixed lipid layer comprises 0.1-10.0 mol % of DSPE-PEG.
[17] The nanoparticle of any one of paragraphs 2-16, wherein the mixed lipid layer comprises no more that 4 mol % of DSPE-PEG.
[18] The nanoparticle of any one of paragraphs 2-17, wherein the mixed lipid layer consists essentially of 5-20 mol % of MPPC or MSPC.
[19] The nanoparticle of any one of paragraphs 2-18, wherein the mixed lipid layer consists essentially of 5-18 mol % of MPPC or MSPC.
[20] The nanoparticle of any one of paragraphs 2-19, wherein the mixed lipid layer consists essentially of 8.5-10 mol % of MPPC or MSPC.
[21] The nanoparticle of any one of paragraphs 2-20, wherein the mixed lipid layer consists essentially of 85-95 mol % of DPPC or DPPG.
[22] The nanoparticle of any one of paragraphs 2-21, wherein the mixed lipid layer consists essentially of 0.1-10.0 mol % of DSPE-PEG.
[23] The nanoparticle of any one of paragraphs 2-22, wherein the mixed lipid layer consists essentially of no more that 4 mol % of DSPE-PEG.
[24] The nanoparticle of paragraph 16, 22, or 23 wherein the DSPE-PEG is DSPE-PEG2000.
[25] The nanoparticle of any one of paragraphs 2-24, wherein the mixed lipid layer forms a lipid bilayer comprising of DPPC or DPPG; MPPC or MSPC; and DSPE-PEG.
[26] The nanoparticle of any one of paragraphs 2-25, wherein the mixed lipid layer forms a lipid bilayer comprising of DPPC, MPPC and DSPE-PEG.
[27] The nanoparticle of any one of paragraphs 2-26, wherein the mixed lipid layer forms a lipid bilayer comprising of DPPC, MPPC and DSPE-PEG in the molar ratio 90:10:4.
[28] The nanoparticle of any one of paragraphs 1-27, wherein the nanoparticle comprises a second inner layer of mixed lipid which encapsulates the aqueous core comprising of the chemokine.
[29] The nanoparticle of paragraph 28, wherein the second layer of mixed lipid comprises of DPPC or DPPG; and MPPC or MSPC.
[30] The nanoparticle of paragraph 28 or 29, wherein the second layer of mixed lipid comprises of DPPC and MPPC.
[31] The nanoparticle of any one of paragraphs 28-30, wherein the second layer of mixed lipid comprises of DPPC and MPPC in the molar ratio 90:10.
[32] The nanoparticle of any one of paragraphs 1-31, wherein the nanoparticle is a temperature-responsive liposome wherein the chemokine in the aqueous core is released from the nanoparticle when the environment of the nanoparticle is between 38° C. and 43° C.
[33] The nanoparticle of any one of paragraphs 1-32, wherein at least 70% of the chemokine is released when the environment of the nanoparticle is between 38° C. and 43° C.
[34] The nanoparticle of any one of paragraphs 1-33, wherein the chemokine is released within 5 minutes when the environment of the nanoparticle is between 38° C. and 43° C.
[35] The nanoparticle of any one of paragraphs 1-34, wherein the nanoparticle is a temperature-responsive liposome wherein at least 70% of the chemokine in the aqueous core is released within 5 minutes when the environment of the nanoparticle is between 38° C. and 43° C.
[36] The nanoparticle of any one of paragraphs 1-35, wherein the aqueous core comprises only one chemokine.
[37] The nanoparticle of any one of paragraphs 1-36, wherein the aqueous core comprises only two chemokines, the two-chemokine combination is selected from the group consisting of CXCL13 and CXCL12; CXCL13 and CCL19; and CXCL12 and CCL19.
[38] The nanoparticle of any one of paragraphs 1-37, wherein the aqueous core comprises all three chemokines CXCL13, CXCL12, and CCL19.
[39] The nanoparticle of any one of paragraphs 1-38, wherein the aqueous core further comprises a fluorescent dye or radioactive dye.
[40] A composition comprising a nanoparticle comprising at least a lipid layer shell and an aqueous core, wherein the nanoparticle wherein the aqueous core comprising at least one chemokine selected from the group consisting of CXCL13, CXCL12, and CCL19, wherein the at least a lipid layer shell encapsulates the aqueous core, and wherein the at least a lipid layer shell has a phase transition temperature between 38° C. and 43° C.
[41] The composition of X, wherein the chemokines selected from the group consisting of CXCL13, CXCL12, and CCL19, and they are recombinant chemokines.
[42] The composition of any one of paragraphs 40-41, wherein the nanoparticle is a liposome.
[43] A composition of a nanoparticle of any one of paragraphs 1-39.
[44] The composition of any one of paragraphs 40-43, further comprising at least one pharmaceutically acceptable carrier, diluent, excipient or adjuvant.
[45] The composition of any one of paragraphs 40-43, further comprising a thermosensitive magnetic liposome (TSML).
[46] The composition of any one of paragraphs 40-43, further comprising GM-CSF.
[47] A method of treating cancer, the method comprising: (a) administering a composition comprising a nanoparticle comprising at least a lipid layer shell and an aqueous core to a subject's preselected tumor or cancer target site in need of treatment for cancer, wherein the aqueous core comprising at least one chemokine selected from the group consisting of CXCL13, CXCL12, and CCL19, wherein the at least a lipid layer shell encapsulates the aqueous core, and wherein the at least a lipid layer shell has a phase transition temperature between 38° C. and 43° C.; and (b) heating the subject's preselected tumor target site to a temperature of between 38° C. and 45° C., whereby the chemokine in the aqueous core is released from when the environment of the nanoparticle is between 38° C. and 43° C.
[48] The treatment method of paragraph 47, whereby the infiltration of natural IgM producing B cells to the tumor/cancer target site is increased by administration of the composition followed by the heating.
[49] The treatment method of paragraph 47 or 48, whereby the released chemokine at the tumor/cancer target site promotes in vivo infiltration of the subject's own natural IgM producing B cells to the tumor/cancer target site.
[50] A method of treating cancer, the method comprising administering: (a) a composition of any of paragraphs 40-46 to a subject in need of treatment for cancer; and (b) heating a subject's preselected tumor target site to a temperature of between 38° C. and 45° C., whereby the chemokine in the aqueous core is released from the nanoparticle when the environment of the nanoparticle is between 38° C. and 43° C.
[51] A method of increasing infiltration of natural IgM producing B cells in a subject to the subject's tumor target site, the method comprising administering: (a) a composition of any of paragraphs 40-46 to the subject; and (b) heating a subject's preselected tumor/cancer target site to a temperature of between 38° C. and 45° C., whereby the chemokine in the aqueous core of the nanoparticle of the composition is released from the nanoparticle when the environment of the nanoparticle is between 38° C. and 43° C.
[52] The treatment method of any of paragraphs 47-51, wherein the preselected tumor target site is a solid tumor.
[53] The treatment method of any of paragraphs 47-52, wherein the administration is by direct intratumoral injection.
[54] The treatment method of any of paragraphs 47-52, wherein the method of administration is by parenteral, oral, buccal, pulmonary, intravenous, intramuscular, subcutaneous, aural, rectal, vaginal, ophthalmic, intradermal, intraoccular, intracerebral, intralymphatic, intraarticular, intrathecal or intraperitoneal injection.
[55] The treatment method of any of paragraphs 47-54, wherein the heating of step (b) is by high intensity focused ultrasound (HIFU) allows non-invasive heating to establish hyperthermia (40-45° C.) of tumor/cancer target site over time.
[56] The treatment method of any of paragraphs 47-55, wherein the subject is a mammal.
[57] The treatment method of paragraph 56, wherein the mammal subject is a primate mammal.
[58] The treatment method of paragraph 56, wherein the mammal is a human.
[59] A method of expanding and/or stimulating natural IgM producing B cells derived from a subject, the method comprising culturing a population of natural IgM producing B cell from a subject with a liposome comprising phosphatidylcholine (PC) and/or a composition comprising a liposome comprising PC for a period of time under culture conditions that promotes the expansion of the initial population of natural IgM producing B cells.
[60] The cell expansion method of paragraph 59, wherein the natural IgM-producing cells are phagocytic B cells.
[61] The cell expansion method of paragraph 59 or 60, wherein the natural IgM-producing cells are B-1 cells.
[62] The cell expansion method of any one of paragraphs 59-61, wherein the natural IgM-producing cells are phagocytic B-1 cells
[63] The cell expansion method of any one of paragraphs 59-62, wherein the natural IgM-producing cells are phagocytic L2pB1 cells.
[64] The cell expansion method of any one of paragraphs 59-63, wherein the culturing is ex vivo.
[65] The cell expansion method of any one of paragraphs 59-64, the method further comprising providing a sample of peritoneal cavity cells from the subject, wherein the sample comprises natural IgM producing B cells.
[66] The cell expansion method of any one of paragraphs 59-65, the method further comprising selecting for natural IgM producing B cells from the subject prior to the ex vivo culturing.
[67] The cell expansion method of any one of paragraphs 59-66, the method further comprising harvesting for natural IgM producing B cells from the cell culture after the ex vivo culturing.
[68] The cell expansion method of any one of paragraphs 59-67, the method further comprising cryopreservation of the harvested natural IgM producing B cells prior to use.
[69] A method of treating cancer, the method comprising administering a population of ex vivo culture expanded natural IgM producing B cells to a subject in need of treatment for cancer, wherein the natural IgM producing B cells are culture expanded by a method of any of paragraphs 59-68.
[70] A method of treating cancer in a subject in need of cancer treatment, the method comprising: (a) culturing an initial population of natural IgM producing B cell with a liposome comprising phosphatidylcholine (PC) and/or a composition comprising a liposome comprising PC for a period of time under culture conditions that promotes the expansion of the initial population of natural IgM producing B cells; (b) culturing the cell ex vivo; and (c) administering the harvested cell to a recipient subject in need of treatment for cancer.
[71] A method of treating cancer, the method comprising administering to a subject in need of treatment for cancer: (a) a composition of any of paragraphs 40-46, whereby the tumor infiltration of IgM producing B cells is increased by administration of the composition; and (b) a cell expanded by a method of any of paragraphs 56-68.
[72] The treatment method of paragraph 71, the method further comprising providing a sample of peritoneal cavity cells from a donor subject, wherein the sample comprises natural IgM producing B cells.
[73] The treatment method of paragraph 70 or 71, the method further comprising a step of selecting for the expanded natural IgM-producing cells prior to administering the cell to the subject.
[74] The treatment method of any one of paragraphs 70-73, the method further comprising a step of harvesting for expanded natural IgM-producing cells prior to administering the cell to the subject.
[75] The treatment method of any one of paragraphs 70-74, the method further comprising a step of enriching for expanded natural IgM-producing cells prior to administering the cell to the subject.
[76] The treatment method of any one of paragraphs 70-75, the method further comprising a step of cryopreserving the expanded natural IgM-producing cells prior to administering the cell to the subject.
[77] The treatment method of any one of paragraphs 70-76, wherein the natural IgM-producing cells are phagocytic B cells.
[78] The treatment method of any one of paragraphs 70-77, wherein the natural IgM-producing cells are B-1 cells.
[79] The treatment method of any one of paragraphs 70-78, wherein the natural IgM-producing cells are phagocytic B-1 cells
[80] The treatment method of any one of paragraphs 70-79, wherein the natural IgM-producing cells are phagocytic L2pB1 cells.
[81] The treatment method of any one of paragraphs 70-80, wherein the natural IgM-producing cell is obtained from a healthy donor subject.
[82] The treatment method of any one of paragraphs 70-81, wherein the natural IgM-producing cell is obtained from peripheral blood; through hemodialysis; from the peritoneal cavity; through peritoneal dialysis; or from a tumor sample.
[83] The treatment method of any one of paragraphs 70-82, wherein the donor subject and the recipient subject are not the same subject.
[84] The treatment method of any one of paragraphs 70-83, wherein the natural IgM-producing cell is non-autologous to the recipient subject.
[85] The treatment method of any one of paragraph s 70-84, wherein the non-autologous natural IgM-producing cell is at the minimum HLA match with the recipient subject.
[86] The treatment method of any one of paragraphs 70-82, wherein the donor subject and the recipient subject are the same subject.
[87] The treatment method of one of paragraphs 70-82, and 86, wherein the natural IgM-producing cell is autologous to the recipient subject.
[88] The treatment method of any one of paragraphs 70-87, wherein the administration is by direct intratumoral injection.
[89] The treatment method of any one of paragraphs 70-88, wherein the method of administration is by parenteral, oral, buccal, pulmonary, intravenous, intramuscular, subcutaneous, aural, rectal, vaginal, ophthalmic, intradermal, intraoccular, intracerebral, intralymphatic, intraarticular, intrathecal or intraperitoneal injection.
[90] The treatment method of any one of paragraphs 70-89, wherein the subject is a mammal.
[91] The treatment method of paragraph 90, wherein the mammal subject is a primate mammal.
[92] The treatment method of paragraph 90 or 91, wherein the mammal is a human.

The technology described herein is further illustrated by the following examples which in no way should be construed as being further limiting.

### EXAMPLES

### Example 1

The immune system is constantly screening and removing pre-cancerous cells, a process known as cancer immuno-surveillance. Immuno-surveillance differs from conventional immune response in that it does not launch systemic inflammation, it is an ongoing maintenance process that is not terminated in a short term and more importantly it has a broad-spectrum cancer recognition mechanism. Most cancer immunotherapy strategies that try to launch conventional immune responses against specific tumor antigen or signal pathway have limited success due to lack of adaptation to cancer variation and short-lived effects. Described herein is an exploitation of the immunosurveillance mechanism to provide a novel robust, broad-spectrum and self-sustainable cancer therapy.

As described herein, the inventors have discovered previously unappreciated immune functions of a subpopulation of B lymphocytes that produce anti-cancer natural IgM, indicating that these cells play critical roles in cancer immuno-surveillance and are more ideal than most immune cells currently used for cancer immunotherapy. Based on the data described herein, provided herein are several novel nanoparticle and natural IgM-producing phagocytic B cell (NIMPAB)-based cellular therapy methods that will evoke a novel robust, sustainable, and non-inflammatory immune response against cancer.

Specifically, contemplated experiments herein are:

Method #1: Intratumoral or intravenous (i.v.) injection of nanoparticles that carry B cell-attracting chemokines to increase tumor-infiltrating NIMPAB cells.

Method #2: In vitro expansion of patient-derived or healthy donor-derived NIMPAB cells followed by adoptive transfer back to patients to boost NIMPAB-mediated inhibition of tumor. Step 1: Patient or donor cell isolation: Natural IgM positive B cells will be isolated from patient peripheral blood (through hemodialysis), peritoneal cavity (through peritoneal dialysis), and surgically removed solid tumor before chemotherapy. Step 2: Cell priming and expansion: Isolated B cells will be co-cultured with phospholid-modified nanoparticles in vitro for varying amount of time and phatocytic B cells will be enriched. Step 3: Cell transfer: Resulting B cells will be washed and prepared for transfer back to patients.

Method #3: Combined therapy of Method #1 and #2 as well as other immunotherapies.

### Example 2: Development of a Cell-Based Immune Therapy Using Nanoparticles and Natural IgM-Producing Phagocytic B Cells (NIMPAB)

Evidence indicates that that natural IgM-producing phagocytic B cells (NIMPAB) play central roles in the immunosurveillance of cancer. Among thousands of antibodies cloned from cancer patients, all antibodies that can distinguish cancer cells from normal cells are germ-line coded natural IgM antibodies, produced by CD5+ B-1 B lymphocytes. These IgM antibodies not only distinguish tumor cells from healthy cells, they can also induce tumor cell death by a process termed lipoptosis. A subset of B-1 B cells that express PD-L2 in mice, termed L2pB1 cells, harbors an antibody repertoire enriched for IgM antibodies that recognize self-antigens, especially phospholipids. Unlike other phagocytes, these L2pB1 B cells specifically phagocytize phospholipid-modified nanoparticles. More importantly, these L2pB1 B cells can phagocytize cancerous cells. It has also been demonstrated that lipids accumulate in dying cancer cells incubated with L2pB1-containing peritoneal cells. Cancer cell death and growth inhibition are diminished when L2pB1 cells are depleted. Therefore, L2pB1 in mice is likely the equivalent of NIMPAB cells in humans.

Despite the above-mentioned mechanisms indicating that NIMPAB cells possess multi-anti-cancer functions, NIMPAB cells have never been explored for cancer immunotherapy. NIMPAB exist in both humans and mice. It is contemplated herein that boosting NIMPAB-mediated immunosurveillance functionality in cancer patients will not only control the existing cancer but also enhance the immunosurveillance that prevents and controls future secondary cancer development in the same patient.

Described herein are nanoparticle methods for intratumoral delivery of NIMPAB-attracting chemokines to enhance tumor-infiltration of NIMPAB cells. Additionally, described herein are lipid nanoparticle methods for in vitro expansion and boosting anti-tumor functions of NIMPAB cells for potential cellular immunotherapy. To facilitate development and testing of such therapies, described herein is a mouse model, where L2pB1 cells can be tracked and/or depleted in vivo.

### Example 3: Enhancing L2pB1 Cell Tumor Infiltration by Intratumoral Injection of Chemokine-Carrying Nanoparticles

Many cytokine and peptide-carrying nanoparticles have been developed to modulate T cells and other cells for various diseases (38). Intra-tumor chemokine expression has also been achieved by adenovirus-mediated expression (39). However, NIMPAB cell-attracting chemokine-carrying nanoparticles have never been synthesized and have never been considered for intra-tumor delivery for cancer immunotherapy

L2pB1 cells reside mostly in body cavities. Several chemokines have been known to induce B-1 cell migration. A knock-in and conditional knock-out mouse model was generated where L2pB1 cells can be tracked and monitored by fluorescent protein expression. B16 melanoma cells can be inoculated into these mice and tumor-infiltrating L2pB1 cells analyzed by immunofluorescence analysis. Intratumoral delivery of L2pB1-attracting chemokines by nanoparticles can enhance L2pB1 cell tumor infiltration. More tumor-infiltrating L2pB1 cells can be detected after intratumoral injection of chemokine-carrying nanoparticles and stronger inhibition of tumor. Intratumoral and i.v. injections of chemokine-nanoparticles can be compared. The purpose of the chemokine-carrying liposomes or nanoparticles is to attract natural IgM, phagocytic B lymphocytes (NIMPAB) into tumors. The delivering method is by direct intra-tumor injection.

Materials and Methods

Generating Chemokine-Carrying Nanoparticles (CCN) or Liposomes

Chemokines: It has been reported that B-1 B cells are preferentially attracted to CXCL13, CXCL12 and CCL19 (42-44). Elevation of these cytokines in the tissue will promote B-1 B cells' migration out of body cavities. Recombinant CXCL13, CXCL12 and CCL19 will be packaged into nanoparticles for injection.

Liposomes will be formulated from a mixture of 1-palmitoyl-2-hydroxy-sn-glycero-3-phosphocholine (MPPC), dipalmitoylphosphocholine (DPPC) and disteaoylphosphoethanolamine conjugated with polyethylene glycol (DSPE-PEG). The molar content of MPPC is at least 10 mol %.

Liposomes will be loaded with different combination of 1-3 of the chemokines: CXCL-13, CXCL12, and CCL19, all of which promote NIMPAB migration.

Liposome preparation: Liposomes containing chemokine(s) are prepared using a two-step process.

Step 1: DPPC:MPPC (molar ratio 90:10) dissolved in chloroform:methanol are mixed with chemokine(s) (lipid:chemokine mass ratio ≥10) dissolved in deionized water. The mixture is agitated mechanically and then allowed to stand for at least five minutes. The aqueous phases separates from the organic phase and is removed, leaving lipid:chemokine particles dispersed in chloroform.

Step 2: DPPC:MPPC:DSPE-PEG (molar ratio 90:10:4) dissolved in chloroform is added to the particle dispersion (outer leaflet lipid:inner leaflet lipid molar ratio ≥2). Aqueous medium (i.e. water or buffered saline) is added, followed by sonication of the mixture. A rotary evaporator is used to remove the chloroform, producing liposomes dispersed in the aqueous medium. The liposomes can be extruded to reduce the mean diameter (target ≤150 nm) and the polydispersity.

Characterization of the physicochemcial properties of the chemokine liposomes.

Liposome size distribution is measured with a particle size analyzer and amount of encapsulated chemokine(s) is measured using a colorimetric technique (i.e. BCA assay kit).

Chemokine release: The liposomes are designed to release their payload when heated (38° C.-43° C.) for at least 60 seconds. A migration/transwell assay will be used to determine the chemokine concentration and heating parameters required for NIMPAB cell attraction.

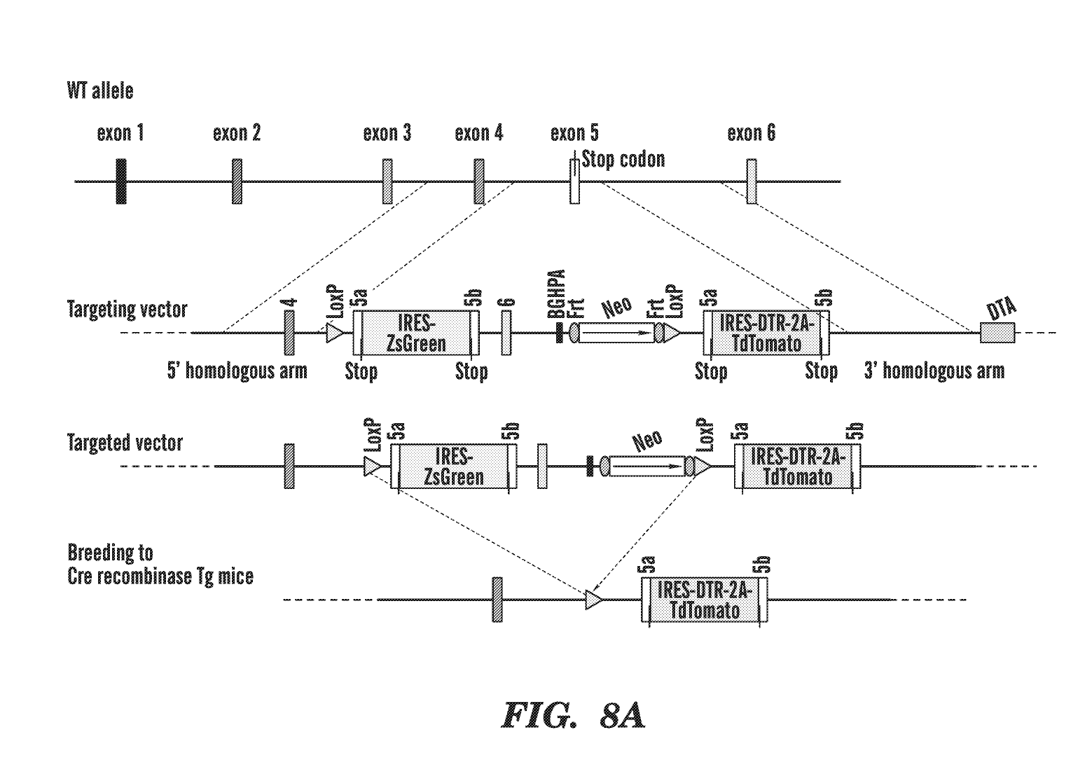

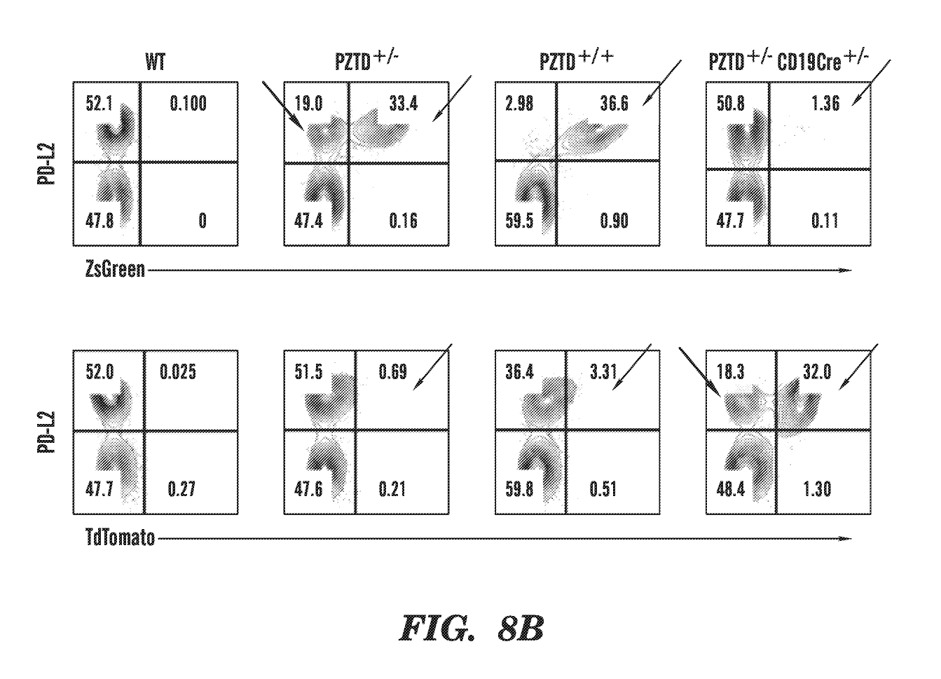

Detecting baseline tumor infiltration of L2pB1 cells. Described herein is a PD-L2-ZsGreen-TdTomato-Diphtheria toxin receptor (PZTD) knock-in and conditional knock-out mouse model, where L2pB1 cells are tracked and monitored by TdTomato fluorescent protein expression (FIGS. 8A-8B). B16F10 melanoma cells are inoculated into these mice and tumor-infiltrating L2pB1 cells analyzed by immunofluorescence analysis of ZsGreen positive cells inside the tissue by FACS, IHC/IF and in vivo imaging. PZTD mice that express ZsGreen florescent protein in L2pB1 cells were injected with 0.5 million B16F10 melanoma cells. Tumors were dissected on day 18 post injection. Single cell suspension of tumor infiltrating lymphocytes (TILs) were obtained by proteolytic dissociation of the tumor mass with gentle collagenase digestion. The lymphocytes were further separated from tumor cells and dead cells by percoll density gradient centrifugation. Lymphocytes were subjected to immunophenotyping by FACS staining with fluorescently-labeled antibodies specific for CD45, CD3e, CD19, B220, IgM, PD-L2. In mice, natural IgM-producing phagocytic B (NIMPAB) cells reside mostly in the peritoneal B1a cell population. B1a cells can be separated into two similar size subpopulations based on the surface expression of PD-L2. L2pB1 subpopulation expresses PD-L2, whereas L2nB1 subpopulation does not. L2pB1 cells are enriched with self-reactivity, hence have the most NIMPAB cells. L2pB1 cells are actively accumulated inside melanoma tumor as compared to lymph nodes and spleen. FACS analysis showed PD-L2+ZsGreen+L2pB1 cells in the transplanted B16F10 melanoma tumor, and also draining the lymph nodes and spleen. The percentage of L2pB1 cells in total B cells is plotted. Significantly more L2pB1 cells were present inside tumor than in draining lymph nodes or spleen (data not shown).

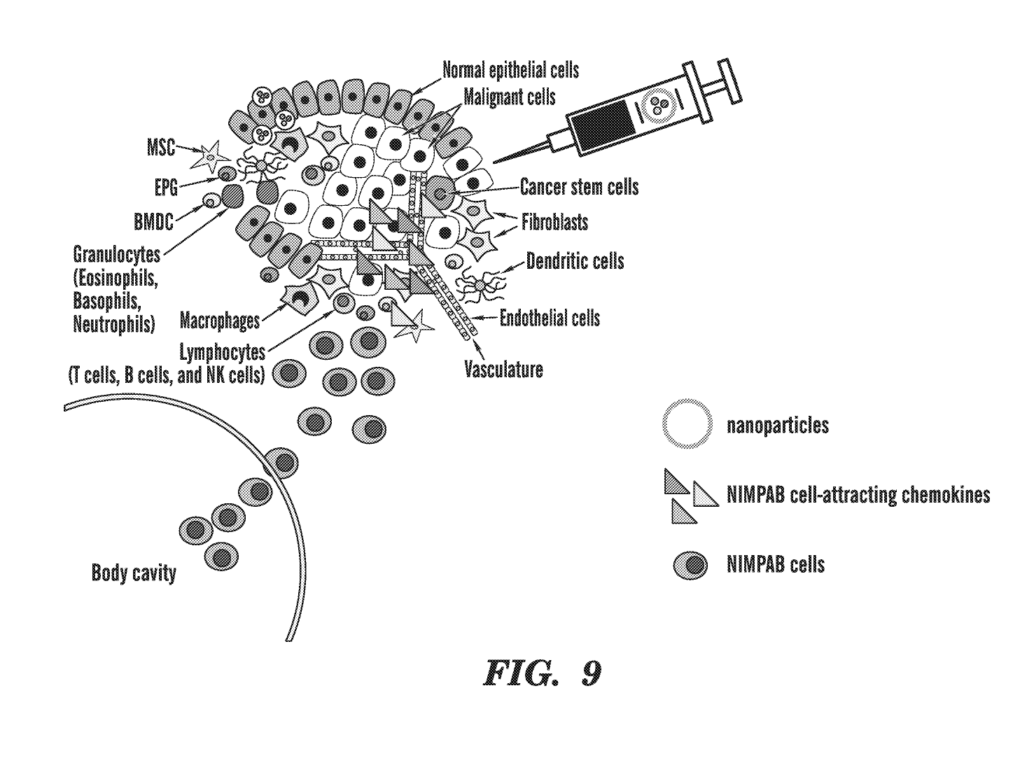

Enhancing tumor infiltration of L2pB1 cells by intratumoral (i.t.) injection of CCN. CCN can be injected, e.g., intratumorally, and the increase of tumor-infiltrating L2pB1 cells evaluated by FACS and immunofluorescent tissue staining. The effects of i.t. injection can be compared with i.v. and i.p. injection. More tumor-infiltrating L2pB1 cells are expected upon i.t. injection of CCNs and complete inhibition of tumor (FIG. 9).

### Example 4: Enhancing Anti-Tumor Functions of L2pB1 Cells by Pre-Incubation with PtC-Nanoparticles

L2pB1 cells are exposed to phosphatidylcholine (PtC)-liposomes and the liposomes are specifically phagocytized. Phospholipid recognition is involved in lipoptosis of cancer cells. Internalization of PtC-liposomes enhanced the natural anti-cancer IgM production and tumor cell phagocytosis by the L2pB1 cells. Tumor inhibition by naive and PtC-liposome-treated L2pB1 cells are observed and compared.

Materials and Method

L-α-phosphatidylcholine Liposomes (PtC Liposomes)

PtC-liposomes can detect B-1 cell phagocytosis and be used as an adjuvant (40, 41). Purpose of the PtC liposomes is for use in identifying and expanding IgM+ phagocytic B (NIMPAB) cells in ex vivo cell cultures. PtC liposomes are ex vivo incubation with cells in culture.

The liposomes are composed of L-α-phosphatidylcholine (PtC), a saturated lipid (i.e. DSPC), and a saturated phospholipid conjugated with polyethylene glycol (PEG). The molar content of PtC in the lipid shell is at least 80 mol %.

Liposome preparation and characterization: Liposomes are prepared via sonication and/or extrusion. The target mean diameter of the liposomes is 300-400 nm. A particle size analyzer will be used to measure liposome size distribution.

For control PtC-coated beads, fluorescent particles can be used.

Fluorescent particles were prepared using a modified Stober process (45). The bead size ranges from 500 to 600 nm. Dye precursor was prepared by reacting Tetramethylrhodamine isothiocyanate (TRITC) dye with (3-Aminopropyl)triethoxysilane (APTS). Rhodamine Green beads were prepared in the same way except that a layer of PtC was spread on the bead surface (46). These are silica nanoparticles used to evaluate and analyze B-1 cell phagocytosis.

Preparation of Green Florescent Beads with pHrodo-Red Dye

Reverse phase evaporation was used to coat green fluorescent silica beads (Discovery Scientific, USA) with a lipid bilayer (inner leaflet: DOTAP; outer leaflet: 85 mol % PtC, 10% pHrodo-Red (P36600, Life Technologies) conjugated to DPPE, and 5% DSPE-PEG2k). The bead size distribution, concentration, and zeta potential were measured with qNano (Izon, Oxford, UK) and dynamic light scattering (90 Plus, Brookhaven). The final product had an average diameter of 530 nm.

In Vitro Assessment of Phagocytosis

Peritoneal cavity (PerC) cells were recovered by injecting HBSS with 2% FBS into the PerC. PBL were harvested from blood with cardiac puncture and RBC lysis. 1×106 cells were incubated overnight at 37° C. with fluorescent beads, e.g., fluorescent Ptc-coated beads, at 10:1 bead-to-cell ratio and 5 μg/ml LPS in supplemented RMPI medium in 96-well plates. Cells were harvested and subjected to confocal microscopy and flow cytometry analysis.

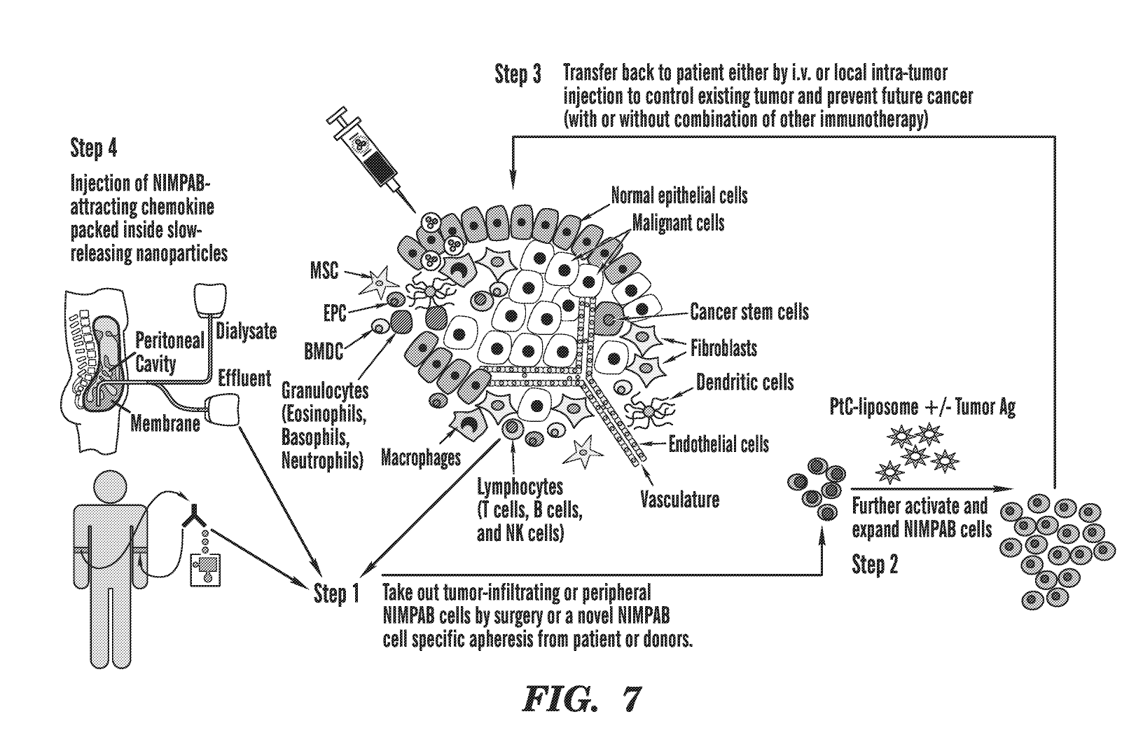

FIG. 7 summarizes the methods of increasing tumor-infiltrating NIMPAB cells and enhancing anti-tumor functions of NIMPAB cells.

Effects of PtC-liposome phagocytosis on L2pB1 natural IgM production. L2pB1 cells can be FACS-sorted and co-cultured with PtC-liposome for 3 days. Supernatant can be collected and anti-IgM ELISA can be performed to measure the IgM level. Supernatant with PtC-liposome, control liposome and no liposome samples can be used for comparison.

Effects of PtC-liposome phagocytosis on L2pB1 proliferation. FACS-sorted L2pB1 cells can be labeled with CFSE, followed by 3-day co-culture with PtC-liposomes. Proliferation of L2pB1 cells can be analyzed by FACS analysis of CFSE florescent intensity, with lower intensity indicating more proliferation. Alternatively, total peritoneal washout cells can be co-cultured with PtC-liposomes. At the end of the culture, FACS analysis can be performed to measure percentage increase of L2pB1 cells between PtC-liposome, control liposome and untreated samples. If PtC-liposomes specifically promote L2pB1 cell proliferation, PtC-liposome co-cultured samples can have the highest percentage increase.

Effects of PtC-liposome phagocytosis on L2pB1 cancer cell phagocytosis. B16F10 melanoma cells can be CFSE-labeled and co-cultured with FACS-sorted L2pB1 cells with or without PtC-liposome pre-treatment. Melanoma cells can be either untreated or pre-treated with 0.4 ug/ml Doxorubicin or 0.6 ug/ml Paclitaxel to increase cell death. FACS analysis can be performed to measure green CFSE engulfment by L2pB1 cells. PtC-liposome pre-treatment enhancement of L2pB1 phagocytosis is indicated by increased CFSE-green fluorescence in pre-treated L2pB1 cells, as compared to untreated L2pB1 cells.

Effects of PtC-liposome phagocytosis on L2pB1 cell induced cancer cell lipoptosis. B16F10 melanoma cells can be co-cultured with PtC-pre-treated L2pB1 cells or untreated L2pB1 cells. Lipoptosis of B16F10 melanoma cells can be measured by oil red O lipid staining.

Effects of PtC-liposome phagocytosis on L2pB1 cell cancer antigen presentation. PtC-liposome treated or untreated L2pB1 cells can be incubated with OVA peptide followed by co-culturing with CD4 T cells isolated from DO11.10 transgenic mice. T cells from DO11.10 TCR-transgenic mice specifically recognize OVA peptides presented by antigen presenting cells and will proliferate. PtC-liposome treated L2pB1 cells promote more T cell proliferation than untreated L2pB1 cells.

Effects of PtC-liposome phagocytosis on L2pB1 cell expression of IL-10, PD-L1, PD-L2, GM-CSF. Expression of IL-10, PD-L1, PD-L2, and GM-CSF on L2pB1 cells can be measured by ELISA, FACS, and RT-PCR after co-culture with PtC-liposome and control liposome.

### Example 5: Combined Cellular Immunotherapy of Adoptive L2pB1 Cell Transfer and Intratum Oral Chemokine-Nanoparticle Injection

L2pB1 cells can be depleted in the above-mentioned animal model. B16F10 melanoma cells can then be inoculated. L2pB1 cells from donor mice can be treated with PtC-liposomes followed by adoptive transfer to the mice bearing the primary B16F10 melanoma. Tumor size can be compared in mice with and without L2pB1 cell transfer. In mice receiving L2pB1 adoptive transfer, the effects of intratumoral chemokine-nanoparticle injection can be determined. In addition to controlling existing tumors, the effect of the combined therapy on preventing the development of future secondary tumors, which are different from the primary tumor, can be determined.

The methods and compositions described herein break new ground in cancer research by exploring the potential of utilizing the immune surveillance system to combat cancer. The novel therapies described herein will significantly advance the cancer immunotherapy field by providing a potential self-renewable therapy for both cancer treatment and cancer prevention. This new therapy addresses current challenges in cancer therapy research, such as lack of sustainability and adaptation to cancer heterogeneity, and will provide a giant leap forward in the cancer immunotherapy field.

Chemotherapy plays a critical role in reducing tumor burdens. However, the toxicity and low-specificity of these agents may cause tremendous damage to patients. Moreover, substantial evidence indicates that chemotherapy may cause therapy-related drug resistance and malignancy (1-5). Worst of all, administrating chemotherapy treatment compromises the immune system and renders patients at higher risk of much severer secondary cancers (6-10).

Immunotherapy presents many advantages over chemotherapy, since it provides lower toxicity and higher specificity. However, current immunotherapy strategies face many challenges, such as resistance inside the suppressive tumor microenvironment, limited responsiveness in one or a few cancer types and systemic inflammation that promote tumor metastasis. Most of all, the effects of immunotherapy may be short-lived and unable to adapt to the heterogeneity of tumors and future cancer development (11-18). These challenges are a critical barrier to progress in the cancer therapy field.

To address the challenges mentioned above, described herein is a revolutionary new immune therapy strategy that utilizes the natural-IgM producing phagocytic B cells (NIMPAB). NIMPABs play central roles in immune surveillance of cancer and are a candidate for a brand new immunotherapy.

The existence of immune surveillance of cancer is evidenced by the increased spontaneous malignancies in both humans and animals with immune deficiency or immune suppression (19, 20). Recent studies also show that higher numbers of tumor-infiltrating-B lymphocytes (TIL-B) are associated with better prognosis (21, 22) and most TIL-B cells recognize glycolipids and oxidized antigens exposed during tumor cell apoptosis (23, 24). These results indicate that immune surveillance of cancer not only works on new, emerging cancers, but also is actively involved in controlling established cancers.

However, the difference between immune surveillance and immune response to cancerous damage is often mixed. In the past decade, studies have shown that immune surveillance of cancer exists before cancer is even developed, and this surveillance is capable of both preventing and controlling cancer. For example, among the thousands of human monoclonal antibodies cloned from various tissues of cancer patients, all cancer-specific antibodies are exclusively germ-line coded natural IgM antibodies produced by CD5⁺ B-1 B lymphocytes (25). These natural IgM antibodies not only distinguish tumor cells from healthy cells, they also induce tumor cell death (26, 27). More importantly, they also exist in healthy donors without any cancer, which indicates that they are components of the immune surveillance system that existed before cancer development. They differ from the immune components that are induced by the inflammatory tissue damage after the establishment of malignancy.

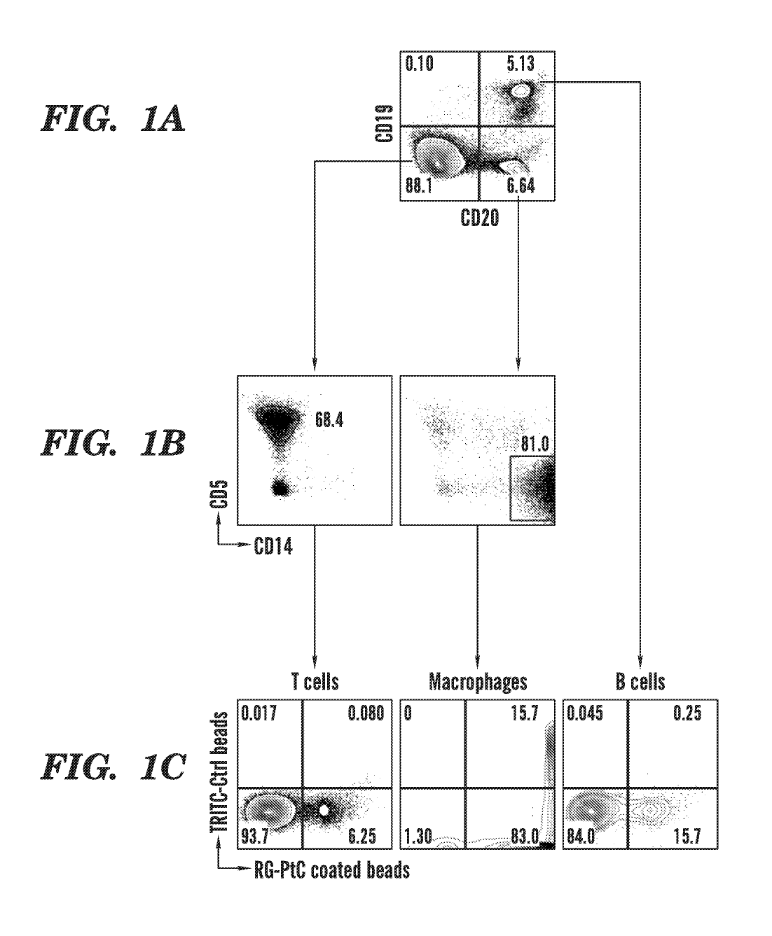

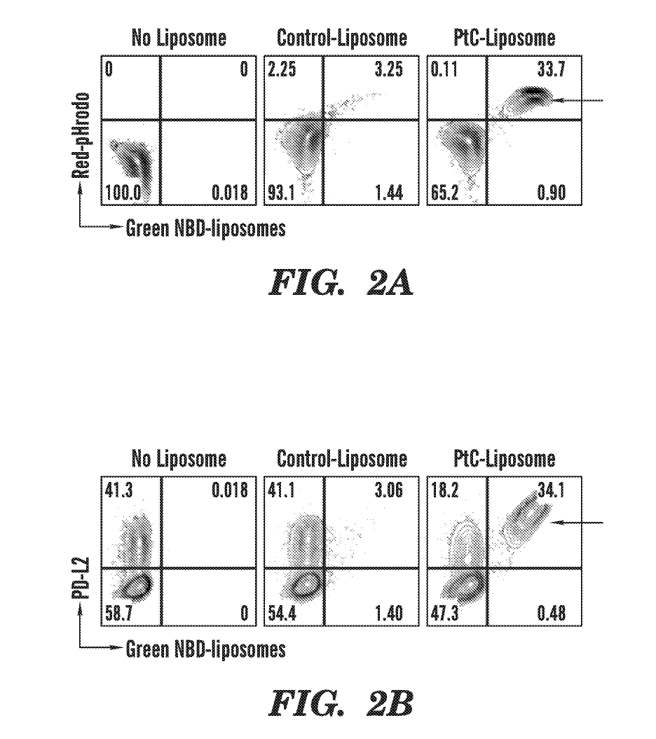

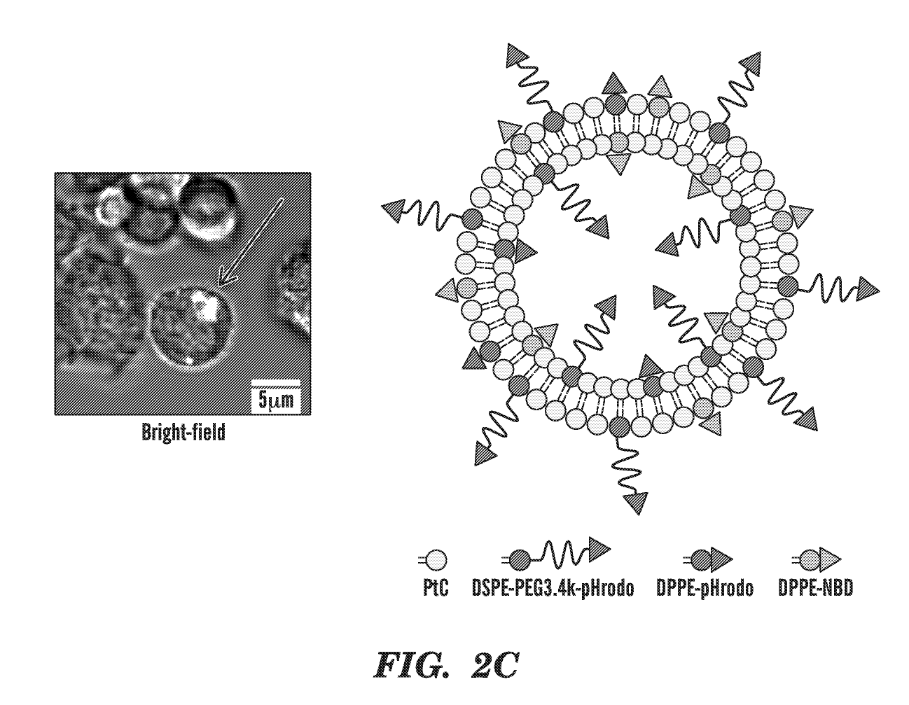

A significant portion of these natural IgM-producing CD5⁺ B-1 B cells in both humans (FIGS. 1A-1C) and mice (FIGS. 2A-2C) have a phospholipid-specific phagocytosis capacity (28). B-1 B cells can directly phagocytize apoptotic cancer cells. Breast cancer cells of the cell line 7367 were grown to 50% confluence on glass-bottom tissue culture dishes. Cancer cells were treated with 0.4 ug/ml Doxorubicin or 0.6 ug/ml Paclitaxel. Peritoneal washout cells (PCW) was collected from WT C57BL/6 mice and stimulated with 1 ug/ml LPS. After 24 hours, cancer cells were labeled with CFSE, whereas PCW cells were stained with CD19-AF405 and CD5-APC. Cancer cells and PCW cells were co-cultured in the presence of LPS for 48 hours and 72 hours. Microscopic analysis was performed. B1 B cells were seen as large blue cells whereas B2 B cells are small round faint blue cells. B1 B cells became plasma/macrophage like cells after 48 hour of stimulation and closely interact with cancer cells (data not shown). Apoptotic bodies and microvesicles were seen at the interaction. Some of them have been engulfed by B-1 B cells (data not shown). B-2 B cells are not actively engaged with any cancer cell.

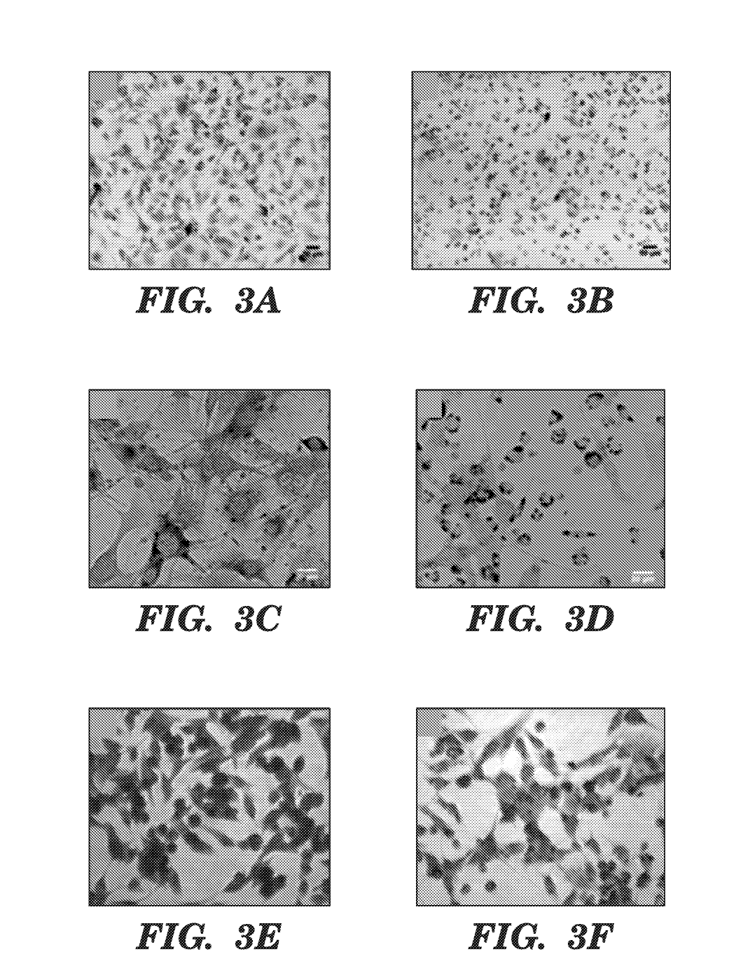

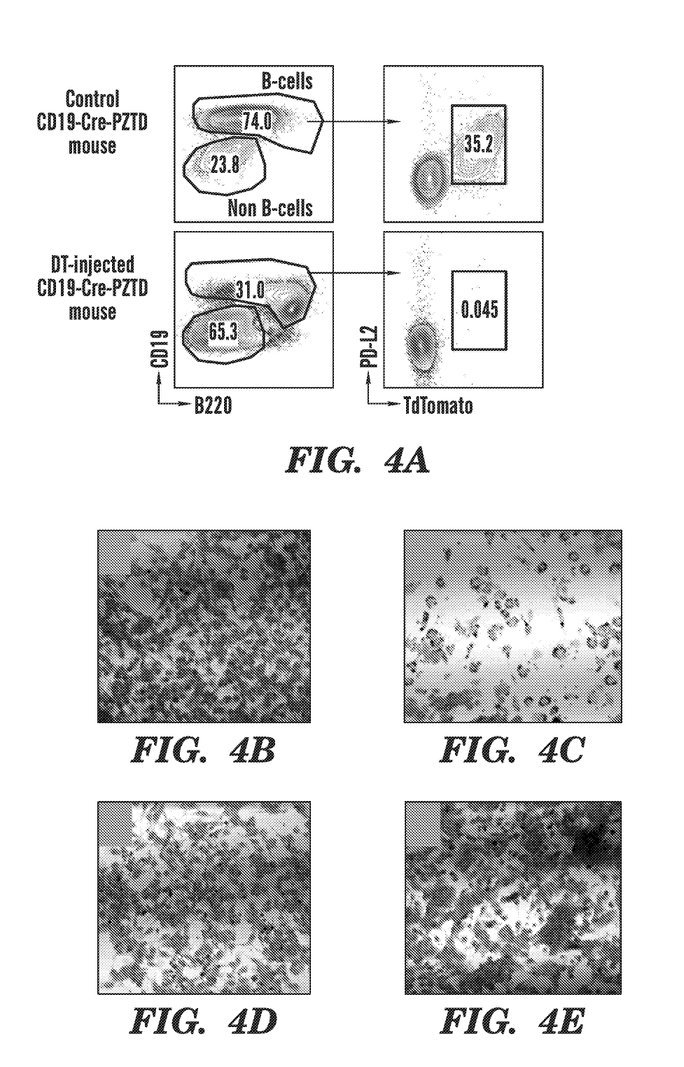

More importantly, our preliminary studies suggest that B-1 B cell-containing peritoneal cells induce cancer cell lipoptosis, apoptosis by lipid-over feeding (FIGS. 3A-3F). Furthermore, depletion of L2pB1 B cells, a PD-L2-expressing subpopulation of B-1 B cells, diminishes lipoptosis and tumor cell inhibition (FIGS. 4A-4E).

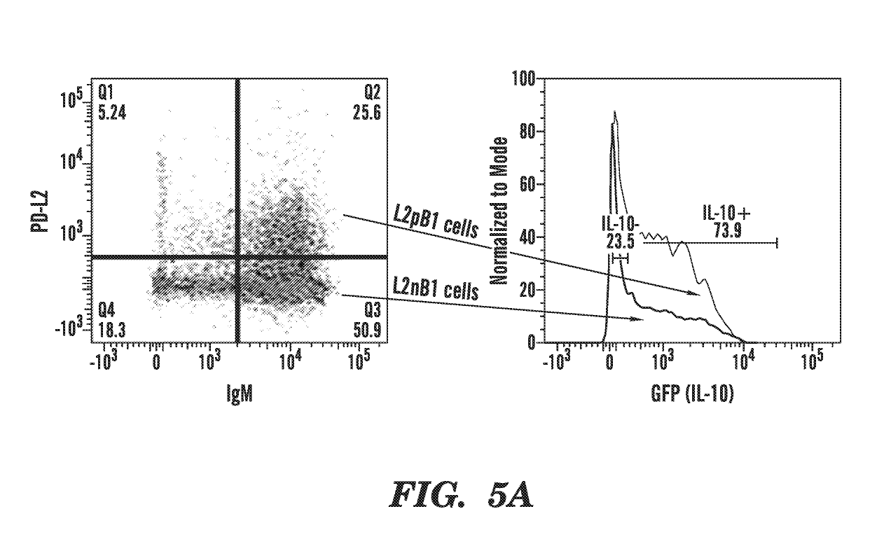

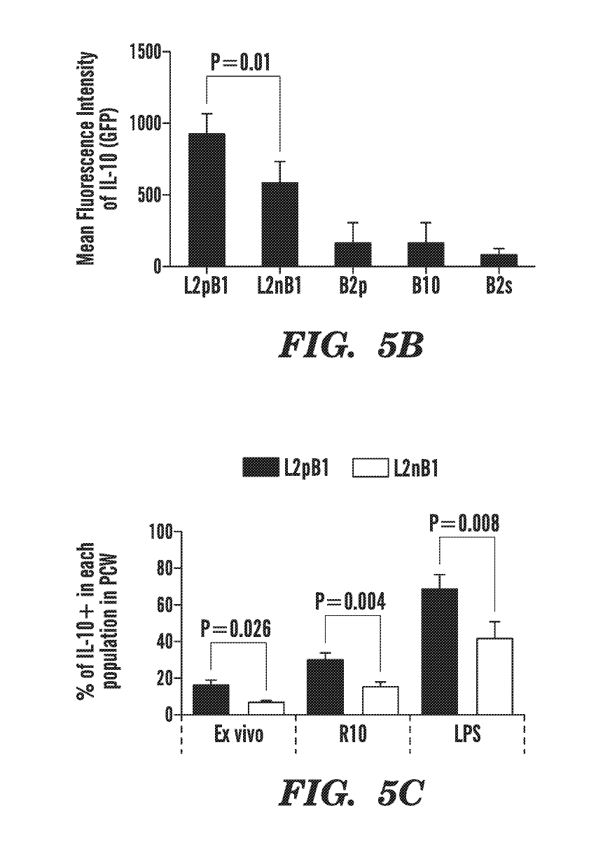

In mice, L2pB1 cells are composed of over 50% of B-1 B cells (29). These cells cannot only phagocytize, but they also constantly express anti-inflammatory cytokine IL-10, more than any other types of B cells (FIGS. 5A-5C). Depletion of L2pB1 cells reduced peritoneal IL-10 level by 5-6 folds. These data indicate that in addition to recognizing and removing cancer cells, L2pB1 cells also tightly regulate inflammation through PD-L2 and IL-10 expression, which might help restrict tumor metastases.

Of further interest, PD-L2 could differentially inhibit exhausted CD8 T cells and Treg cells in the tumor while sparing fresh CD8 T cells (30). Thus, it is contemplated herein that L2pB1 cells can reverse the immune-suppressive microenvironment of the tumor by regulating T cell populations through PD-L2 and PD-1 signaling. In addition, B-1 cells are also capable of expressing GM-CSF upon activation (31). The anti-cancer effect of GM-CSF is well documented (32). However, the efficiency of administering GM-CSF for clinical treatment is limited and hard to control. It is reasonable to postulate that L2pB1 cells may produce GM-CSF upon activation and regulate the tumor microenvironment with more precise adjustment of local levels of cytokine and localized control. In supporting the use of B-1 cells in an anti-cancer role, Azevedo et al. reported that these cells play important roles in concomitant tumor immunity (33). In addition, Leyva et al. reported during the Merinoff World Congress on B-1 cell Development and Function, that CDS+ peritoneal B-1a cells and their IgMs are required for protection in a peritoneal cancer model (34).

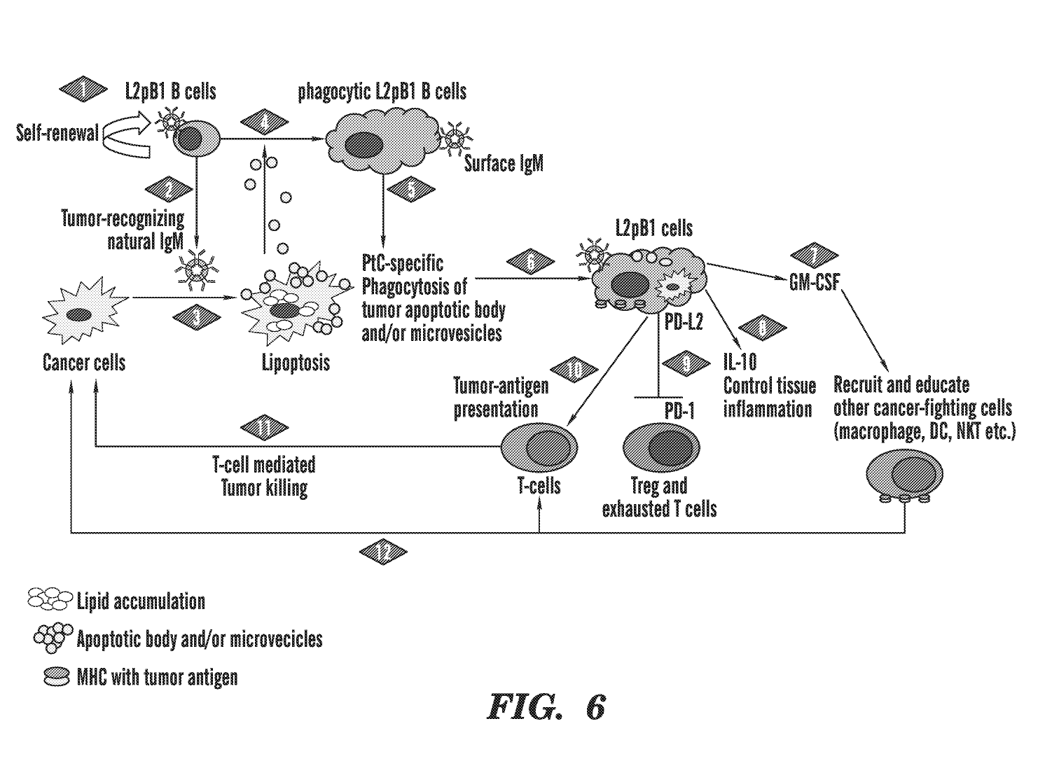

In summary, NIMPAB cells, like L2pB1 cells in mice, possess multiple anti-cancer functions (FIG. 6). These include 1) a whole naturally existing antibody repertoire specific for cancer cell recognition and killing, 2) direct phagocytosis capacity, and 3) regulatory checkpoint ligands and cytokines. Therefore, the potential of NIMPAB cells has never been fully appreciated as a key player in cancer immune surveillance. With such complete anti-cancer and regulatory functions, NIMPAB cells are superior candidates for a revolutionary NIMPAB-based cellular immunotherapy.

Among all the immunotherapies that are undergoing clinical trials, two treatment methods are very promising. One is using human monoclonal IgM antibodies cloned from cancer patients (35, 36). The other is using patient-derived tumor-infiltrating T lymphocyte (TIL-T) (37). Both of the strategies utilize the immune systems' own “weapon” (IgM) and “soldiers” (T cells). The method described herein, permit utilization of the “mastermind” and “army general” of our immune surveillance system. The NIMPAB cells not only are the source of the “weapon”, i.e. cancer-targeting IgMs, they also are also the “soldiers” themselves that kill tumor cells when needed. More importantly, they have the capacity to “instruct” (tumor antigen presentation) and “regulate” (activate and inhibit) different T cell subsets and other TILs through MHC-tumor antigen complex, PD-L1, PD-L2, IL-10 and potentially GM-CSF. By boosting these NIMPAB cells using a PtC-nanoparticle and subsequently putting them back into patients or mobilizing more NIMPAB into the tumor, a “command center” is established, greatly enhancing the cancer treatment. This is a paradigm-shifting advance. The present strategy differs from the common immunotherapy design by building up a sustainable “command center” that not only tackles existing tumor growth, but will also recognize and inhibit future cancer lesions.

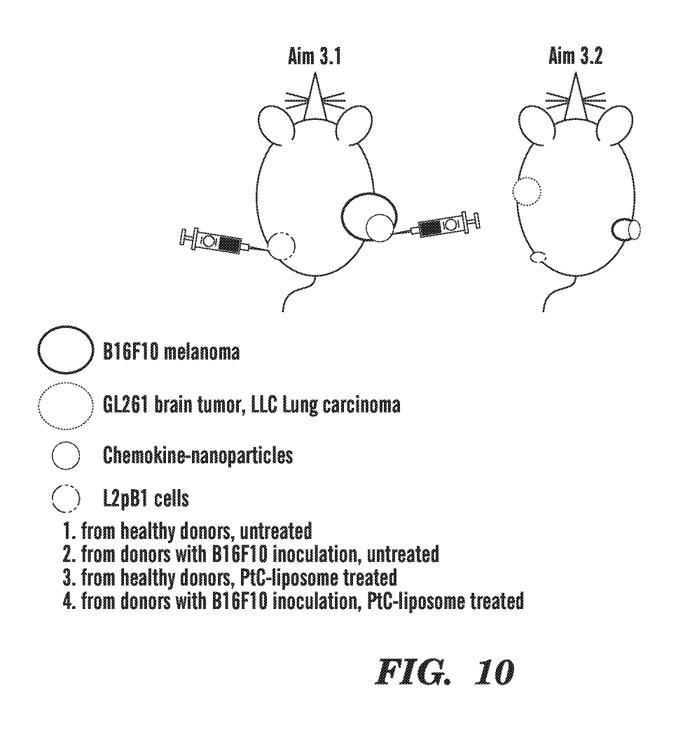

Inhibition of primary tumor. B16F10 melanoma cells can be inoculated in CD19-Cre-PZTD mice with or without L2pB1 cell depletion. After tumor size reaches 0.5 cm³, mice can receive i.p. injection of L2pB1 cells followed by i.t. injection of CCN (FIG. 10). L2pB1 cells from donor mice can be treated with PtC-liposomes followed by adoptive transfer to the mice bearing the primary B16 melanoma. Tumor size can be compared in mice with and without L2pB1 cell transfer.

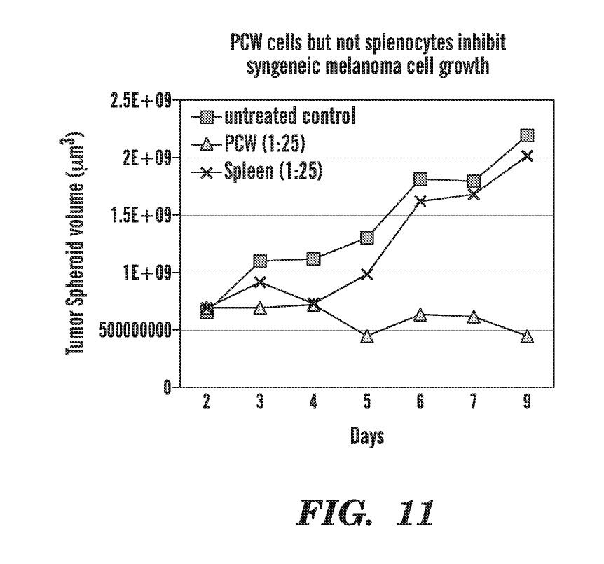

Prevention of secondary tumor. In addition to controlling existing tumors, the combined therapy strategy can also be assessed for ability to prevent the development of future secondary tumors that are either the same or different from the primary one. B16F10 melanoma cells that are the same as the primary tumor, GL261 brain tumor cells or LLC Lung carcinoma cells that are different than the primary tumor, can be inoculated on the opposing site of the primary tumor inoculation site respectively (FIG. 11). Tumor size and a full analysis of tumor-infiltrating cells can be obtained at different time points with or without combined L2pB1 and CCN treatment. L2pB1-CCN combined treatment enhances immune surveillance, such that either a full block of secondary tumor growth (both same and different than primary tumor) or a certain level of inhibition can be observed.

The methods described herein shift the paradigm of effector-immune cell-based immunotherapy towards an immunosurveillance-based immunotherapy. By way of non-limiting example, patients' NIMPAB cells could be cryopreserved before chemotherapy. Alternatively, if some patients are NIMPAB-deficient, they would receive a NIMPAB transfer from healthy donors. As NIMPAB can self-renew, a blood bank of NIMPAB can be established from healthy donors or any individual at a young age to use in case the cells are needed when the respective donor becomes older.

Novel concept of “soldiers and weapons” vs. “commander” in immunotherapy. The approach described herein is designed to maximally utilize the body's own immune system by boosting the key regulator of immune surveillance. This is very different from most current immune therapy strategies. If current cancer immunotherapy strategies are conceptualized as engineering new weapons (antibodies, cytokines etc.) to target cancer, or putting cancer-fighting soldiers (T cells, NK, DCs) back to the field (patients), the strategy described herein is putting a mastermind or an army general back to the battlefield. Weapons need to be controlled by soldiers. Soldiers need constant replacement upon exhaustion. Neither weapons nor frontline soldiers have a big picture of the cancer battlefield and thus are not efficient in adaptation to changing situations. The general knows and is in charge of the soldiers, is capable of making the best use of the weapons and will regulate the soldiers as they adapt to various emerging situations. Based on this concept, the approach described herein is designed to revitalize the general, i.e. NIMPAB, and eventually control the existing cancer and prevent future development of new cancer growth.

Natural immune-surveillance versus inducible inflammatory immune response. In contrast to current immune therapy strategies, described herein is the utilization of a very different set of immune components led by NIMPAB cells. These B cells and antibodies were born to control cancerous cells. Unlike other anti-cancer immune components activated after cancerous growth has been established, NIMPAB exists before the onset of cancer and has almost all the anti-cancer features, as compared with other immune cells (Table 1). The methods described herein address the double-edge dilemma of the pro- and anti-cancer inflammation in current approaches. By utilizing NIMPAB cells with broad and highly cancer-specific targeting through natural IgM-mediated lipoptosis and PtC-specific phagocytosis, there will be minimal damage to healthy tissues through IL-10 and PD-L2 mediated regulation. These NIMPAB cells will not only regulate tissue inflammation, they also will revitalize T cells by inhibiting exhausted T cells and Treg cells, which are the main cause of tumor microenvironment immune suppression and drug resistance.

### Example 6: Depletion of L2pB1 Cells from Peritoneal Cavity Reduced the Inhibition Efficacy of Peritoneal Cavity Washout (PCW) Cells on Tumor Spheroid Growth

B16F10 melanoma cells were cultured in spheroid ultra-low attachment microplate to form 3D spheroids. Calcein AM (which is imaged as green fluorescent) and PI (which is imaged as red fluorescent) staining were used to evaluate tumor spheroid formation with the increase of PI-positive necrotic core, which starts to appear on day 4. Calcein AM positive live cells were observed as a typical green ring around the spheroid (data not shown). Tumor spheroids were co-incubated with the same number of PCW cells and splenocytes respectively. Tumor spheroid size was measured using Celigo image cytometer. Tumor growth was calculated as tumor size over time.

In the presence of PCW cells but not splenocytes, tumor growth was inhibited (FIG. 11). Uniform 3D tumor spheroids were formed by day 4 after seeding. Then wild type PCW and splenocytes were added to the spheroids. L2pB1-depleted PCW (PCW-DT) and splenocytes (SP-DT) were both obtained from transgenic mice injected with Diphtheria toxin (DT), and the PCW-DT and SP-DT were added to the spheroids. After 48 hours co-culture, tumor spheroids were imaged and the tumor spheroid sizes (volume) were noted. PCW treated spheroids were shrunk after 48 hours whereas less shrinkage were observed in spheroids co-cultured with PCW-DT. No significant changes were observed in splenocytes treated spheroids (FIG. 11).

### Example 7: Depletion of L2pB1 Cells In Vivo Resulted in Enlarged Melanoma

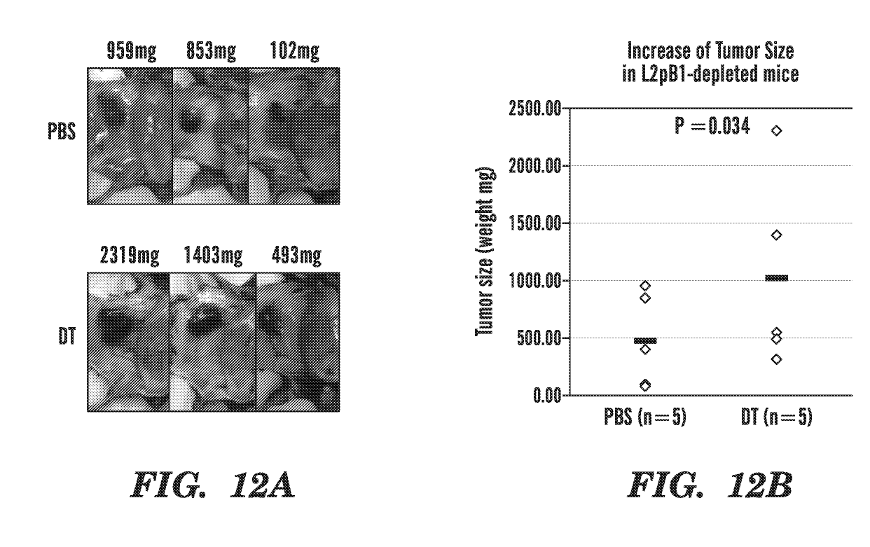

CD19-Cre-PZTD transgenic mice received i.p. injection of PBS or diphtheria toxin (DT) for 4 days before they were inoculated with 106 B16F10 melanoma cells. Mice were sacrificed 10-14 days post inoculation. Tumors were imaged and dissected for weight measurement (FIG. 12A). Depletion of L2pB1 cells was evaluated by FACS analysis of the peritoneal cavity washout cells. Depletion of L2pB1 cells ranged from 70% to 80% in DT-injected mice was seen at end point compared to PBS-injected mice. DT-injected mice show significant increase of tumor size and tumor weight as well as angiogenesis in L2pB1-depleted mice (FIG. 12B).

### Example 8: CXCL13 is the Optimal Chemokine that can Attract and Mobilize Mouse PCW Cells and Preferably L2pB1 Cells

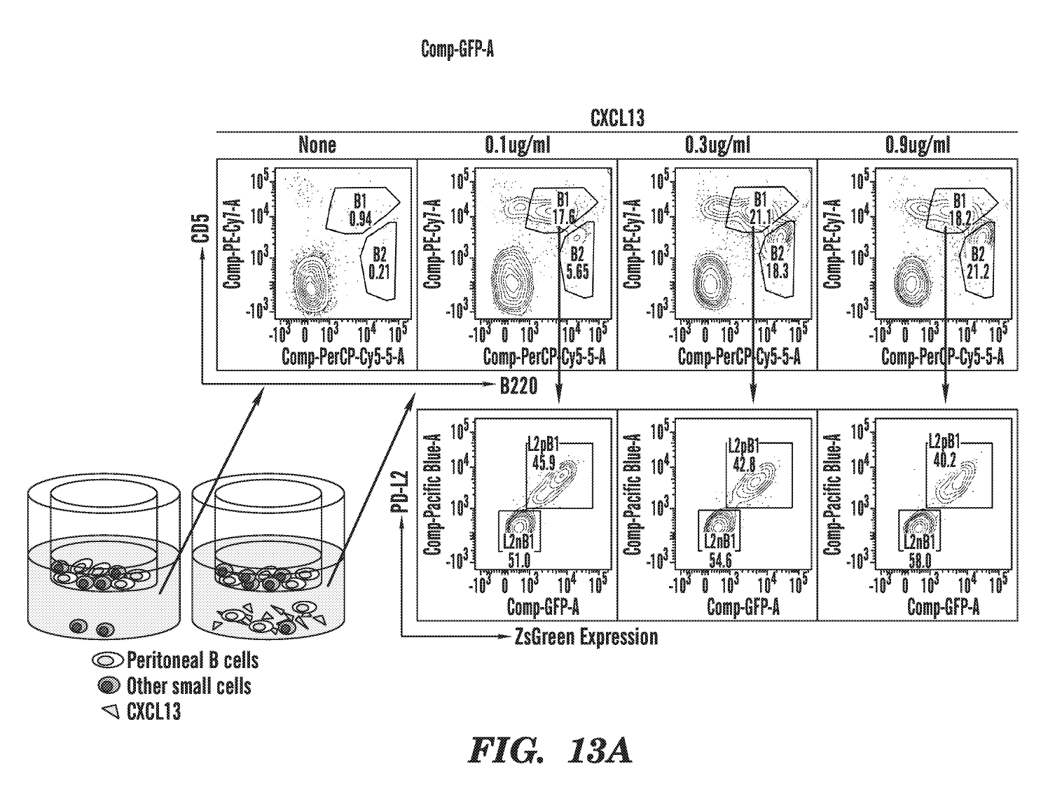

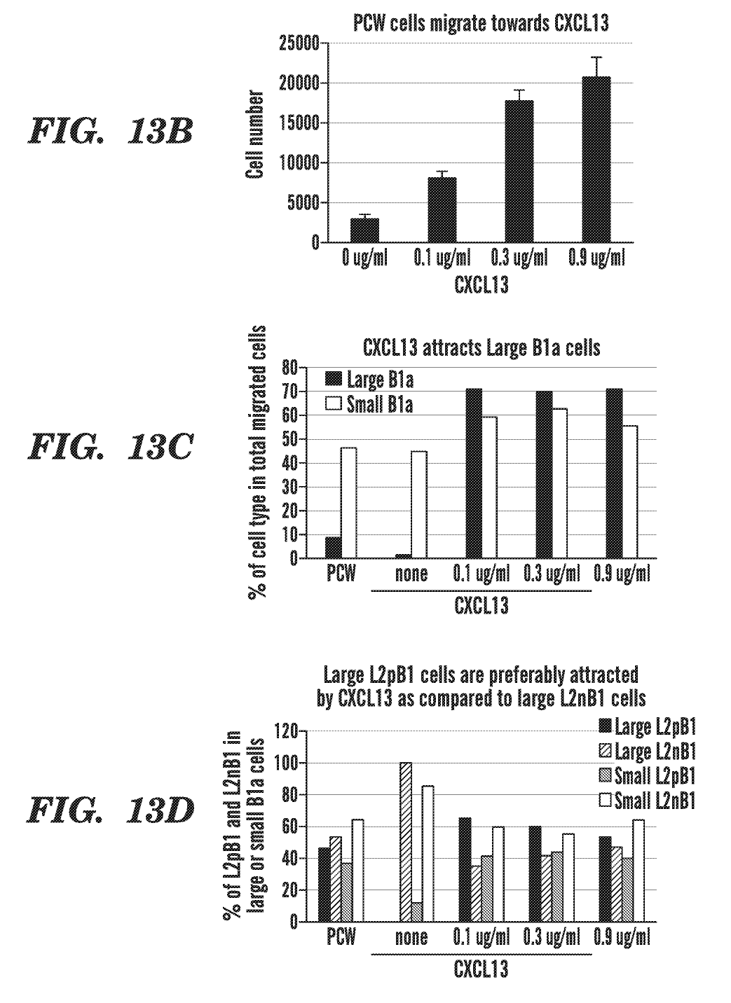

The inventors show here that B1a cells were preferably attracted by low concentration of CXCL13. Mouse peritoneal washout cells (mPWC) were isolated and placed on the top chamber of the transwell (FIG. 13A). Medium alone or medium containing different concentrations of CXCL13 (0.1, 0.3, 0.9 ug/ml) was placed at the bottom chamber of the transwell plate. Cells were then incubated for 3 hours before being counted on the Celigo Image cytometer. All samples were placed in at least triplicate wells. After counting, cells at the bottom chambers of the transwells were harvested and pooled for FACS analysis on LSRII flow cytometer. Very few B220+CD5+ B1a cells were present in the bottom chamber in the absence of CXCL13. Significant increase of B1a cells were obtained from the bottom chambers in the presence of CXCL13. Therefore, CXCL13 preferably attract B1a cells at lower concentration whereas B2 B cells were attracted at higher concentration of CXCL13.

The inventors also show here that PCW cells migrate towards CXCL13 (FIG. 13B). In a similar transwell experiment, CXCL13 mobilize large B1a cells (FIG. 13C). As a control, total peritoneal cavity washout (PCW) cells were analyzed directly after isolation from mouse. Percentage of large B1a cells and small B1a cells were noted. Small B1a cells composed more than 40% of the total lymphocytes in PCW cells, whereas large B1a cells composed about 10% of the total lymphocytes in PCW cells. When there is no CXCL13 treatment, very few PCW cells migrated to the bottom chamber (data not shown). Among those few that migrated, most of them were small B1a cells. In the presence of CXCL13, there is a significant increase of large B1a cell number in the bottom chamber (data not shown).

The inventors also show among the three known chemokines for B1 cells, CXCL13 attracts more large L2pB1 cells than L2nB1 cells (FIG. 13D). There were almost equal percentage of L2pB1 cells and L2nB1 cells in the PCW. In the absence of CXCL13, among the few cells that migrated into the bottom chamber, neither large nor small L2pB1 cells were present, whereas most of the L2nB1 cells were there, likely through free fall. In the presence of CXCL13, there was a significant increase of L2pB1 cells migrated toward bottom chamber (data not shown).

### Example 9: CXCL13-Carrying Temperature Sensitive Liposomes (TSL) can Attract PCW Cells Upon Increase of Temperature from 37° C. to 44° C.

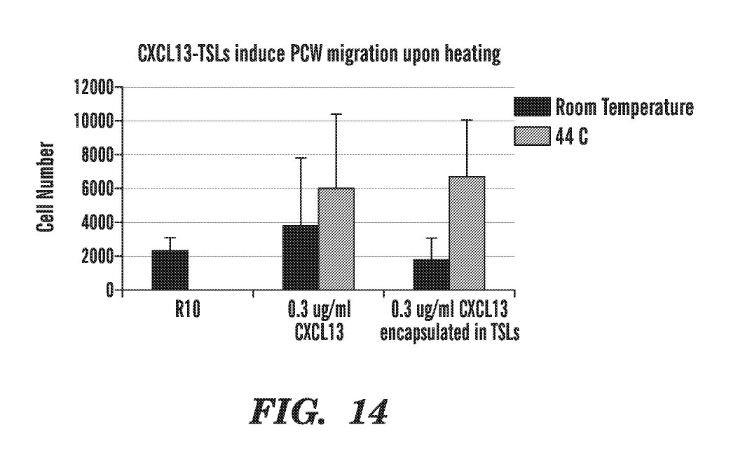

In order to assess the ability of the temperature-sensitive liposomes (TSLs) to release CXCL13 upon thermal triggering, additional transwell migration assays were performed. Three conditions were evaluated, medium only, 0.3 ug/ml CXCL13 and 0.3 ug/ml CXCL13-loaded TSLs. Prior to loading into the bottom chamber of the transwells, the TSLs remained at room temperature or heated to 44° C. for 20 minutes. As described in Example 8, mouse peritoneal washout cells were isolated and placed on the top chamber of the transwells. Then the cells were incubated at 37° C. for 3 hours in the above 3 conditions. Triplicates were performed for each condition. The number of cells migrated to the bottom chamber of the transwell was determined by using the Celigo Microwell Plate Imager. For TSLs incubated at room temperature prior to loading into the transwells, there is comparable migration as that for the control media. However, when the TSL were heated at 44° C., there was an increase in cell migration compared to room temperature and control samples. This indicates the successful release of CXCL13 from the TSLs and that these CXCL13 retains chemotaxis function (FIG. 14).

### REFERENCES

1. Krishnan B, Morgan G J. Cancer Epidemiol Biomarkers Prev. 2007; 16(3):377-80.
2. Kamat N, et al. Cancer Cell Int. 2014; 14(1):118. PMCID: 4260186.
3. Wong T N, et al. Nature. 2015; 518(7540):552-5. PMCID: 4403236.
4. Travis L B, et al. J Natl Cancer Inst. 2000; 92(14):1165-71.
5. Travis L B, et al. N Engl J Med. 1999; 340(5):351-7.
6. Farnault L, et al. Clin Dev Immunol. 2012; 2012:421702. PMCID: 3415262.
7. Barrett A J, Savani B N. Leukemia. 2009; 23(1):53-8. PMCID: 4170943.
8. Ferrari S, et al. Oncol Rep. 2002; 9(5):1107-13.
9. Solomayer E F, et al. Clin Cancer Res. 2003; 9(1):174-80.
10. Zandvoort A, et al. Clin Immunol. 2003; 107(1):20-9.
11. Zarour H M, Ferrone S. Eur J Immunol. 2011; 41(6):1510-5. PMCID: 3426035.
12. Guinn B A, et al. Mol Ther. 2007; 15(6):1065-71.
13. Chiriva-Internati M, et al. Int Rev Immunol. 2011; 30(2-3):67-70.
14. Liu B, et al. J Hematol Oncol. 2010; 3:7. PMCID: 2831814.
15. Rescigno M, et al. Biochim Biophys Acta. 2007; 1776(1):108-23.
16. Ferrantini M, International meeting “Immunotherapy of cancer: challenges and needs”. Cancer Immunol Immunother. 2007; 56(4):581-5.
17. Yee C. J Transl Med. 2005; 3(1):17. PMCID: 1131930.
18. Mesa C, Fernandez L E. Immunol Cell Biol. 2004; 82(6):644-50.
19. Keast D. Lancet. 1970; 2(7675):710-2.
20. Urban J L, Schreiber H. Prog Exp Tumor Res. 1988; 32:17-68.
21. Loi S, et al. J Clin Oncol. 2013; 31(7):860-7.
22. Woo J R, et al. J Transl Med. 2014; 12:30. PMCID: 3914187.
23. Kotlan B, et al. J Immunol. 2005; 175(4):2278-85.
24. Hansen M H, et al. Proc Natl Acad Sci USA. 2001; 98(22):12659-64. PMCID: 60110.
25. Brandlein S, et al. Cancer Res. 2003; 63(22):7995-8005.
26. Vollmers H P, Brandlein S. Methods Find Exp Clin Pharmacol. 2005; 27(3):185-91.
27. Vollmers H P, et al. Adv Drug Deliv Rev. 2006; 58(5-6):755-65.
28. Hung Vo, et al. Immunity, Inflammation and Disease. 2015; 2(4):254.
29. Zhong X, et al. Eur J Immunol. 2007; 37(9):2405-10.
30. Mkrtichyan M, et al. J Immunol. 2012; 189(5):2338-47.
31. Weber G F, et al. J Exp Med. 2014; 211(6):1243-56. PMCID: 4042649.
32. Thorne S H. Immunotherapy. 2013; 5(8):817-9.
33. Azevedo M C, et al. Immunobiology. 2014; 219(5):357-66.
34. Marcela Leyva K H. Tarrytown House Estate and Conference Center 2014.
35. Hensel F, et al. Oncol Rep. 2014; 31(3):1059-66. PMCID: 3926647.
36. Rasche L, et al. Haematologica. 2015.
37. Rosenberg S A, et al. Clin Cancer Res. 2011; 17(13):4550-7. PMCID: 3131487.
38. Swartz M A, et al. Sci Transl Med. 2012; 4(148):148rv9.
39. Guiducci C, et al. J Immunol. 2004; 172(7):4026-36.
40. Vo H, et al. Inflammation and Disease. 2014; 2(4):254-61. PMCID: 4386919.
41. Cruz-Leal Y, et al. International Immunology. 2014; 26(8):427-37.
42. Ansel K M, et al. Immunity. 2002; 16(1):67-76.
43. Forster R, et al. Nat Rev Immunol. 2008; 8(5):362-71.
44. Moon H, et al. J Korean Med Sci. 2012; 27(1):27-35. PMCID: 3247770.
45. Bele, M., et al. 2002. J. Colloid Interface Sci. 254:274-282.
46. Mackiewicz, M. R., et al. 2008. Nanotechnology 19:115607.

TABLE 1
Anti-cancer features of NIMPAB-based immune therapy
in comparison with other cellular immunotherapies

| | Cytotoxic | NK | | |
|---|---|---|---|---|
| Current cellular immunotherapy | T cell | cell | DC | NIMPAB |
| Anti-cancer Natural IgM production | X | X | X | ✓ |
| Tumor-specific recognition | ✓ | ✓ | X | ✓ |
| Direct tumor killing | ✓ | ✓ | X | ✓ |
| Tumor antigen-presentation | X | ? | ✓ | ✓ |
| Direct tumor phagocytosis | X | ? | ? | ✓ |
| Inflammation control | X | X | ? | ✓ |
| Modulate other immune cells in | ✓ | ? | ✓ | ✓ |
| tumor | | | | |

What is claimed is:

1. A nanoparticle comprising at least a lipid layer shell and an aqueous core, wherein the aqueous core comprising at least one chemokine selected from the group consisting of CXCL13, CXCL12, and CCL19, wherein the at least a lipid layer shell encapsulates the aqueous core, and wherein the at least a lipid layer shell has a phase transition temperature between 38° C. and 43° C.

2. The nanoparticle of claim 1, wherein the lipid layer is a mixed lipid layer comprising two or more lipids, or two or more phospholipids.

3. (canceled)

4. The nanoparticle of claim 2, wherein the phospholipids is selected from the group consisting of phosphatidyl cholines, phosphatidyl glycerols, phosphatidyl inositols, phosphatidyl ethanolamines, dipalmitoylphosphatidylcholine (DPPC), 1-palmitoyl-2-hydroxy-sn-glycero-3-phosphocholine (MPPC), 1-myristoyl-2-stearoyl-sn-glycero-3-phosphocholine (MSPC); 1,2-dimyristoyl-sn-glycero-3-phosphocholine (DMPC), 1,2-Dimyristoyl-sn-glycero-3-phosphorylglycerol (DMPG); 1,2-Distearoyl-sn-glycero-3-phosphoethanolamine (DSPE); 1,2-Dioleoyl-sn-glycero-3-phosphocholine (DOPC); 1,2-Dioleoyl-sn-glycero-3-phosphoethanolamine (DOPE); 1,2-dipalmitoyl-sn-glycero-3-phospho-(1′-rac-glycerol) (DPPG); 1,2-Distearoyl-sn-glycero-3-phosphocholine (DSPC); disteaoylphosphoethanolamine conjugated with polyethylene glycol (DSPE-PEG); phosphatidylserine (PS), phosphatidylethanolamine (PE), phosphatidylglycerol (PG), and phosphatidylcholine (PC).

5. (canceled)

6. The nanoparticle of claim 2, wherein the phospholipid is a lysolipid selected from the group consisting of monoacylphosphatidyl cholines, monoacylphosphatidyl glycerols, monoacylphosphatidyl inositols and/or monoacylphosphatidyl ethanolomines.

7. (canceled)

8. The nanoparticle of any one of claim 2, wherein the lipid is selected from the group consisting of DPPC, MPPC, PEG, DMPC, DMPG, DSPE, DOPC, DOPE, DPPG, DSPC, DSPE-PEG, MSPC, cholesterol, PS, PC, PE, and/or PG,

9.-11. (canceled)

12. The nanoparticle of claim 1, wherein the mixed lipid layer comprises or consists essentially of at least one of the group selected from 5-20 mol % of MPPC or MSPC; 5-18 mol % of MPPC or MSPC; 8.5-10 mol % of MPPC or MSPC; 85-95 mol % of DPPC or DPPG; 0.1-10.0 mol % of DSPE-PEG; and no more that 4 mol % of DSPE-PEG.

13.-26. (canceled)

27. The nanoparticle of claim 1, wherein the mixed lipid layer forms a lipid bilayer comprising of DPPC, MPPC and DSPE-PEG, and optionally, wherein the molar ratio is 90:10:4.

28. The nanoparticle of claim 1, wherein the nanoparticle comprises a second inner layer of mixed lipid which encapsulates the aqueous core comprising of the chemokine.

29.-31. (canceled)

32. The nanoparticle of claim 1, wherein the nanoparticle is a temperature-responsive liposome wherein the chemokine in the aqueous core is released from the nanoparticle when the environment of the nanoparticle is between 38° C. and 43° C.

33.-44. (canceled)

45. The composition of claim 40, further comprising a thermosensitive magnetic liposome (TSML), and/or GM-CSF.

46. (canceled)

47. A method of treating cancer, the method comprising:
a. administering a composition comprising a nanoparticle comprising at least a lipid layer shell and an aqueous core to a subject's preselected tumor or cancer target site in need of treatment for cancer, wherein the aqueous core comprising at least one chemokine selected from the group consisting of CXCL13, CXCL12, and CCL19, wherein the at least a lipid layer shell encapsulates the aqueous core, and wherein the at least a lipid layer shell has a phase transition temperature between 38° C. and 43° C.; and
b. heating the subject's preselected tumor target site to a temperature of between 38° C. and 45° C., whereby the chemokine in the aqueous core is released from when the environment of the nanoparticle is between 38° C. and 43° C.

48.-58. (canceled)

59. A method of expanding and/or stimulating natural IgM producing B cells derived from a subject, the method comprising culturing a population of natural IgM producing B cell from a subject with a liposome comprising phosphatidylcholine (PC) and/or a composition comprising a liposome comprising PC for a period of time under culture conditions that promotes the expansion of the initial population of natural IgM producing B cells; wherein the culturing is ex vivo.

60. The cell expansion method of claim 59, wherein the natural IgM-producing cells are selected from the group consisting of phagocytic B cells; B-1 cells; phagocytic B-1 cells; and phagocytic L2pB1 cells.

61.-64. (canceled)

65. The cell expansion method of claim 59, the method further comprising providing a sample of peritoneal cavity cells from the subject, wherein the sample comprises natural IgM producing B cells.

66. The cell expansion method of claim 59, the method further comprising selecting for natural IgM producing B cells from the subject prior to or after the ex vivo culturing.

67.-69. (canceled)

70. The method of treating cancer of claim 47, the method further comprising:
a. culturing an initial population of natural IgM producing B cell with a liposome comprising phosphatidylcholine (PC) and/or a composition comprising a liposome comprising PC for a period of time under culture conditions that promotes the expansion of the initial population of natural IgM producing B cells; and
b. culturing the cell ex vivo; and also
administering the harvested cell to a recipient subject in need of treatment for cancer.

71.-92. (canceled)
